(19) United States
(12) Patent Application Publication
(10) Pub. No.: US 2026/0021061 A1
(43) Pub. Date: Jan. 22, 2026

(54) ARIADNE AND ANALOGS FOR THE TREATMENT OF NEUROLOGICAL AND NEUROMUSCLAR DISORDERS

(71) Applicant: The Trustees of Columbia University in the City of New York, New York, NY (US)

(72) Inventors: Dalibor Sames, New York, NY (US); Prashant Khirsariya, New York, NY (US); David Lankri, New York, NY (US); Michael Cunningham, New York, NY (US)

(73) Assignee: The Trustees of Columbia University in the City of New York, New York, NY (US)

(21) Appl. No.: 19/093,152

(22) Filed: Mar. 27, 2025

Related U.S. Application Data
(63) Continuation of application No. PCT/US2023/075420, filed on Sep. 28, 2023.
(60) Provisional application No. 63/377,524, filed on Sep. 28, 2022.

(51) Int. Cl. A61K 31/137; A61K 31/277; A61K 31/337; A61K 31/695; A61P 25/16

(52) U.S. Cl. CPC A61K 31/137; A61K 31/277; A61K 31/337; A61K 31/695; A61P 25/16

(57) ABSTRACT

The present invention provides method of treating a subject afflicted with a movement disorder, comprising administering to the subject a compound having the structure:

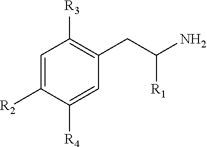

wherein
R₁ is —(C₂-C₁₂ alkyl), -(alkenyl), -(alkynyl), -(cycloalkyl);
R₂ is H, halogen, —NO₂, —CN, —CF₃, —CF₂H, —CH₂OCH₃, —CF₂CH₃, —CF₂OCH₃, -(alkyl), -(alkenyl), -(alkynyl), -(cycloalkyl), -(cycloalkylalkyl), -(heteroalkyl), -(heterocycle), -(heterocycloalkyl), -(aryl), -(heteroaryl), -(hydroxyalkyl), -(haloalkyl), -(alkylaryl), —OH, —O-(alkyl), —O-(alkenyl), —O-(alkynyl), —O-(haloalkyl), —O-(aryl), —O-(heteroaryl), —OCF₃, SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —N-(alkyl)₂, NH-(aryl), —NH-(heteroaryl), —CO₂H, —CO₂-(alkyl), —C(O)—NH₂, —C(O)—NH-(alkyl), —C(O)—NH-(aryl), —SO₂CH₃ or —Si(CH₃)₃;
R₃ is —OCH₃, —OCH₂CH₃, —F or —Cl; and
R₄ is —OCH₃, —OCH₂CH₃ or —SCH₃;
wherein when R₁ is —CH₂CH₃, R₃ is —OCH₃, and R₄ is —OCH₃, then R₂ is other than H, —CH₃, —CH₂CH₃, —CH₂CH₂CH₃, —CH(CH₃)₂, —CH₂CH₂CH₂CH₃, —CH₂OH, —CH(OH)CH₃, —OH, —OCH₂CH₃, —OCH₂CH₂CH₃, —OCH(CH₃)₂, —SCH₃, —SCH₂CH₃, —SCH₂CH₂CH₃, —NO₂, —NH₂, —F, —Cl, —Br or —I,
or a pharmaceutically acceptable salt thereof.

This application is a continuation of PCT International Application No. PCT/US2023/075420, filed Sep. 28, 2023, claiming the benefit of U.S. Provisional Application No. 63/377,524, filed Sep. 28, 2022, the contents of each of which are hereby incorporated by reference into the subject application.

Throughout this application, various publications are referenced, including referenced in parenthesis. The disclosures of all publications mentioned in this application are hereby incorporated by reference in their entireties into this application.

### BACKGROUND OF THE INVENTION

Natural substances with hallucinogenic properties have been used by humans for millennia and likely played important roles in shaping cultural traditions of community integration, healing, spirituality, and religion. In more recent history, since the human bioassay-guided isolation of the first hallucinogen mescaline, the interest in psychedelics has followed an oscillatory pattern with periods of intense activity. Currently the public, scientific, and medicinal interest in psychedelics is on the rise. This is driven by several factors, including the growing mental health epidemic, the limited efficacy of current medications, promising clinical results with prototypical psychedelics, existential despair in diverse communities, and interest in advancing spiritual connectedness, wellness and consciousness understanding. The biological effects of classic psychedelics such as mescaline, DMT, psilocybin and LSD are largely mediated by serotonin receptors. Specifically, induction of transient altered states of consciousness and hallucinosis by these substances is mediated by activation of the serotonin 2A (5-HT2A) receptors (Nichols, 2016) Currently and historically, there are two standing questions in the field of psychedelic research: 1) Which therapeutic effects and modalities are facilitated by psychedelic experiences, and 2) Can substantial therapeutic effects be achieved by psychedelic-related compounds that lack the hallucinogenic activity?

Identification of psychedelic substances via isolation from natural sources or creation by synthesis provided a plethora of structural analogs that exhibit a varying degree of psychoactivity in humans (Nichols, 2017). A systematic search for non-hallucinogenic psychedelic analogs have begun shortly after the discovery of LSD at Sandoz. This effort was guided by the concept of identifying ergoline derivatives with serotonin blocking effects or serotonin receptor antagonists (Hofmann, 2009). However, some of the ergoline compounds, for example lisuride or ergotamine, were later shown to act as 5-HT2A receptor agonists in vitro, while showing no psychedelic activity in humans, introducing a distinct class of pharmacological agents: non-hallucinogenic 5-HT2A agonists. While lisuride is a well-established example, its main therapeutic effect is mediated by the potent dopamine receptor agonism. Contribution of the 5-HT2A receptor modulation in vivo to lisuride's therapeutic efficacy is not known due to its complex polypharmacology.⁴

In this application we focus on a much lesser-known example of non-hallucinogenic psychedelic analog—Ariadne (FIG. 1A). This compound, 2-amino-1-(2,5-dimethoxy-4-methylphenyl)-butane, created and named by Alexander Shulgin, belongs to the mescaline lineage of phenylalkylamine psychedelics. It is closely related to an established synthetic psychedelic, 2,5 Dimethoxy-4-methylamphetamine (DOM), differing only by one methylene group in the alpha-position to the amine (Weingartner, et al., 1971). Shulgin also named Ariadne as 4C-D or “four-carbon DOM”, for the number of carbons of the alkyl chain between the aromatic ring and amine. This naming system is convenient and we here elect to use it as it has already been broadly adopted for his more famous 2C-D (“two-carbon DOM”) series of analogs, such as 2C-D, 2C-B, 2C-T-7 etc.⁶

In contrast to DOM, which is approximately 100-times more potent than mescaline in humans (per oral administration), Ariadne was reported to lack the hallucinogenic effects (Shulgin et al., 2020). In addition to reports from the psychonautic sphere, Bristol-Myers Company, a respectable pharmaceutical house in the U.S., has conducted clinical trials with the (R)-enantiomer of Ariadne in the 1970's (designated as BL-3912A). These studies confirmed the lack of hallucinogenic effects up to and beyond 100 mg per day, notably including patients with psychosis (Partyka et al., U.S. Pat. No. 4,105,695, 1978). Further, Bristol-Myers reported remarkable therapeutic effects including rapid remission of psychotic symptoms in patients suffering from schizophrenia and bipolar disorder (50-100 mg per day), relaxation in catatonics and increased sociability in anxious subjects, as well as nearly complete remission of symptoms in subjects with Parkinson's disease (100 mg per day). Improvement in alertness, mobility and sociability was also reported in geriatric senile subjects (50 mg per day). Pro-cognitive effects (increased learning capacity) was also observed in these clinical trials (Partyka et al., U.S. Pat. No. 4,105,695, 1978). Unfortunately, the actual clinical data were never disclosed. According to Alexander Shulgin, (R)-Ariadne completed phase II of clinical trials with a large number of patients, but further development was halted allegedly due to strategic economic considerations (Shulgin, 1987).

Bristol-Myers has reported preclinical characterization of Ariadne using a multifactorial scale in cats, to assess Ariadne's hallucinogenic-like profile in comparison to (R)-DOM. These results agree with the clinical findings by showing a low combined score of DOM-like behavioral features elicited by Ariadne (e.g., hissing, clawing, catatonia, piloerection, miosis, salivation, arched posture, and muscle rigidity) (Standridge et al., 1980). The low hallucinogenic-like effect of Ariadne enantiomers was confirmed in a rabbit hyperthermia assay (Standridge et al., 1976). Ariadne also showed lack of stimulant effects compared to (S)-amphetamine, exhibiting a distinct behavioral profile from that of classic hallucinogens and psychostimulants in rats and conscious beagle dogs (Buyniski et al., 1974 and Tilson et al., 1977). In the same dose range (10 mg/kg, s.c. or i.p.), (R)-Ariadne improved avoidance behavior in the absence of stimulant-like effects in rats. In rat drug discrimination assays, Ariadne substituted responding in LSD trained animals in one study, in another showed full substitution for MDMA stimulus (Glennon et al., 1993 and Karlsen et al., 2014). However, the molecular pharmacology of Ariadne has remained unknown. A rat smooth muscle spasmogenicity assays suggested a weak serotonergic-like effect of Ariadne compared to serotonin and (R)-DOM (Stanridge et al., 1976).

### SUMMARY OF THE INVENTION

The present invention provides method of treating a subject afflicted with a movement disorder, comprising administering to the subject a compound having the structure:

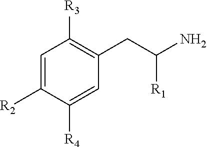

wherein
R₁ is —(C₂-C₁₂ alkyl), -(alkenyl), -(alkynyl), -(cycloalkyl);
R₂ is H, halogen, —NO₂, —CN, —CF₃, —CF₂H, —CH₂OCH₃, —CF₂CH₃, —CF₂OCH₃, -(alkyl), -(alkenyl), -(alkynyl), -(cycloalkyl), -(cycloalkylalkyl), -(heteroalkyl), -(heterocycle), -(heterocycloalkyl), -(aryl), -(heteroaryl), -(hydroxyalkyl), -(haloalkyl), -(alkylaryl), —OH, —O-(alkyl), —O-(alkenyl), —O-(alkynyl), —O-(haloalkyl), —O-(aryl), —O-(heteroaryl), —OCF₃, SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —N-(alkyl)₂, NH-(aryl), —NH-(heteroaryl), —CO₂H, —CO₂-(alkyl), —C(O)—NH₂, —C(O)—NH-(alkyl), —C(O)—NH-(aryl), —SO₂CH₃ or —Si(CH₃)₃;
R₃ is —OCH₃, —OCH₂CH₃, —F or —Cl; and
R₄ is —OCH₃, —OCH₂CH₃ or —SCH₃;
wherein when R₁ is —CH₂CH₃, R₃ is —OCH₃, and R₄ is —OCH₃, then R₂ is other than H, —CH₃, —CH₂CH₃, —CH₂CH₂CH₃, —CH(CH₃)₂, —CH₂CH₂CH₂CH₃, —CH₂OH, —CH(OH) CH₃, —OH, —OCH₂CH₃, —OCH₂CH₂CH₃, —OCH(CH₃)₂, —SCH₃, —SCH₂CH₃, —SCH₂CH₂CH₃, —NO₂, —NH₂, —F, —Cl, —Br or —I,
   or a pharmaceutically acceptable salt thereof.

### BRIEF DESCRIPTION OF THE DRAWINGS

FIGS. 1A-B Ariadne is a non-hallucinogenic analog of phenylalkylamine psychedelics with remarkable efficacy signals in clinical studies. (a) The mescaline molecular lineage of Ariadne, which contains an ethyl group in the alpha-position to the amine. The units represent an approximate measure of psycho-activity in humans relative to mescaline based on subjective reports after per oral administration. In contrast to DOM, a potent psychedelic, Ariadne showed no hallucinogenic effects at doses that induced notable clinical effects. The present work introduces novel analogs of Ariadne. (b) Broad receptor screen (SafetyScreen44, Eurofins-Panlabs) identified the serotonin 5-HT2A and 2B receptors as the only hits above the set threshold (more than 50% displacement of standard radioligands at 10 uM concentration of (rac)-Ariadne). TMA=3,4,5-trimethoxyamphetamine; TMA-2=2,4,5-trimethoxyamphetamine; DOM=2,5-dimethoxy-4-methyl-amphetamine.

FIGS. 2A-C Ariadne is neither a hSERT nor hDAT inhibitor. (A) Ariadne is depicted graphically as not a hSERT inhibitor (IC₅₀>50 μM). Imipramine, a potent hSERT inhibitor, was utilized as the comparative positive control (IC₅₀=21±3 nM). (B) Ariadne is additionally not a hDAT inhibitor (IC₅₀>50 μM) and was compared to a potent hDAT inhibitor, Indatraline (23±3 nM). Inhibition of Ariadne, Imipramine, and Indatraline is presented as normalized fluorescence uptake (uninhibited−inhibited)±SEM and compiled from four separate experiments. (C) Alpha-propyl-Ariadne (5C-D) induced HTR response that is indistinguishable from vehicle; DOPR showed minor to no effects on locomotion, trending toward an increased locomotion at its HTR peak dose (1 mg/kg), consistent with minor stimulatory effects of phenylalkylamine psychedelics in mice.

FIGS. 3A-C 5-HT receptor binding and functional screening of Ariadne and its enantiomers. (a) Structures and binding affinities of Ariadne and enantiomers to 5-HT2A. (b) Heat map of (±)-Ariande and (R)-Ariande in the 5-HTome BRET G protein dissociation assays. Values represent Log (EMAX/EC50) from at least 2 independent experiments (see Supplemental Table X) (c) Comparison of 5-HT2A/2B/2C receptor Gq dissociation and Ca2+ flux activities. Data represent average and S.E.M from three independent experiments performed in triplicate. All data were normalized to percent 5-HT response. (d) Summary of activity data in a tabular form. Data represent average and S.E.M from three independent experiments performed in triplicate.

FIGS. 4A-D Comparison of hallucinogenic (DOx) and non-hallucinogenic (4C-X) analogs in Gq dissociation and βarrestin2 recruitment signaling assays. All data represent average and S.E.M from three independent experiments performed in triplicate. All data are normalized to % 5-HT response. (a-c) 4-methyl-, propyl- and iodo-substituted compounds show a shift toward lower potency and efficacy when alpha-methyl substituent is extended to ethyl. (d) Trend toward lower potency and efficacy continues as alpha-substituent is extended to the propyl group.

FIGS. 5A-C 4-postion analogs of Ariadne compared at 5-HT2 receptors. (a) The methyl, trifluoromethyl, propyl, cyclopropyl and methoxymethyl analogs of Ariadne. (b) comparison of potency and selectivity of 4-substitued analogs. (c) Data summary in a tabular form. Data represent average and SEM from three independent experiments performed in triplicate. All data are normalized to percent 5-HT response.

FIGS. 6A-H In vivo pharmacology of Ariadne and its analogs in comparison to hallucinogen DOPR. (A) Mouse head twitch response (HTR) comparison of psychedelic (rac)-DOPR and non-psychedelic (rac)-Ariadne and its enantiomers. (B) Time course of HTR events in vehicle, DOPR and (R)-Ariadne treated mice. Green band shows the time period used for HTR scoring for dose-response studies. (C) Brain and plasma pharmacokinetic evaluation of Ariadne. Green band shows the time period used for HTR scoring for dose-response studies. (D) HTR dose response curves for DOPR, Ariadne and its analogs. (E) (R)-Ariadne-induced HTR is blocked by MDL100907, 5-HT2A antagonist. (F) Mouse 5-HT2A Gq-dissociation for serotonin, DOPR, (R)-Ariadne and its 4-trifluoromethyl analog, 4C-TFM. (G) Mouse open-field assay for evaluation of (R)-Ariadne's effect on novelty induced locomotion. (H) Mouse open-field assay for DOPR. All drugs administered subcutaneously.

FIGS. 7A-D Effect of Ariadne in behavioral conflict paradigms. (a) Survival plot of effect of (R)-Ariadne (10 mg/kg) on latency to feed in the novelty suppressed feeding (NSF) test 7 days post a single drug administration. (b) Effect of 6 mg/kg and 10 mg/kg (R)-Ariadne on latency to feed in the novelty suppressed feeding (NSF) test 7 days post a single drug administration. (c) Effect of (R)-Ariadne and (±)-DOPR on the time spent in the open vs closed arms and (d) corresponding arm entries 30 minutes and 4 hours post a single subcutaneous injection.

FIGS. 8A-B Comparison of 5-HT2A (a) and 5-HT2B (b) receptor in IP1 formation assay for selected compounds, performed at Cerep, Eurofins.

FIGS. 9A-D Wildtype (WT) and vehicle controls preceding (R)-Ariadne treatment, where all the mice were subjected to the balance beam, hind limb clasping and open field behavior tests (pre-treatment group). (a) Balance beam test, measured in number of runs/minute and (b) time taken to cross the beam (b) Number of runs auxilin KOs could perform in a minute were significantly less compared to WT mice before (R)-Ariadne treatment. (c) Though not significant, auxilin KOs also showed a trend toward an increase in clasping duration. (d) open field test to check distance traveled in given time, which was significantly deteriorated in auxilin KOs before (R)-Ariadne treatment

FIGS. 10A-E Effect of (R)-Ariadne in Auxilin-knockout mouse model of Parkinson's Disease. (a) Effect of (R)-Ariadne on number of balance beam runs performed over 60 seconds, grouped animal averages. (b) Effect of (R)-Ariadne on number of balance beam runs performed over 60 seconds, individual animal traces. (c) Effect of R-Ariadne on average time per run on balance beam. (d) Effect of R-Ariadne treatment on clasping duration. (e) Effect of R-Ariadne treatment on hindlimb clasping score.

### DETAILED DESCRIPTION OF THE INVENTION

Each embodiment disclosed herein is contemplated as being applicable to each of the other disclosed embodiments. Thus, all combinations of the various elements described herein are within the scope of the invention.

Considering the unknown mechanism of action, and our long-standing interest in psychoactive substances, we set out to examine the pharmacological profile of Ariadne in vitro and in mice. Therefore, we investigated Ariadne at many targets, including the serotonin receptors, which are typically involved in the expression of psychedelic effects. We examined Ariadne and its analogs compared to DOM, its structural homolog, in serotonin receptor signaling assays, and head-twitch response and behavioral assays in mice. Guided by these investigations, we provide the first molecular mechanistic hypothesis for the clinical therapeutic signals and the lack of hallucinogenic effects of this compound.

### EMBODIMENTS OF THE INVENTION

The present invention provides a method of treating a subject afflicted with a movement disorder, comprising administering to the subject a compound having the structure:

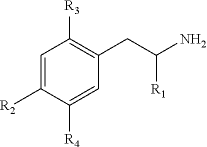

wherein
R₁ is —(C₂-C₁₂ alkyl), -(alkenyl), -(alkynyl), -(cycloalkyl);
R₂ is H, halogen, —NO₂, —CN, —CF₃, —CF₂H, —CH₂OCH₃, —CF₂CH₃, —CF₂OCH₃, -(alkyl), -(alkenyl), -(alkynyl), -(cycloalkyl), -(cycloalkylalkyl), -(heteroalkyl), -(heterocycle), -(heterocycloalkyl), -(aryl), -(heteroaryl), -(hydroxyalkyl), -(haloalkyl), -(alkylaryl), —OH, —O-(alkyl), —O-(alkenyl), —O-(alkynyl), —O-(haloalkyl), —O-(aryl), —O-(heteroaryl), —OCF₃, SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —N-(alkyl)₂, NH-(aryl), —NH-(heteroaryl), —CO₂H, —CO₂-(alkyl), —C(O)—NH₂, —C(O)—NH-(alkyl), —C(O)—NH-(aryl), —SO₂CH₃ or —Si(CH₃)₃;
R₃ is —OCH₃, —OCH₂CH₃, —F or —Cl; and
R₄ is —OCH₃, —OCH₂CH₃ or —SCH₃;
wherein when R₁ is —CH₂CH₃, R₃ is —OCH₃, and R₄ is —OCH₃, then R₂ is other than H, —CH₃, —CH₂CH₃, —CH₂CH₂CH₃, —CH(CH₃)₂, —CH₂CH₂CH₂CH₃, —CH₂OH, —CH(OH) CH₃, —OH, —OCH₂CH₃, —OCH₂CH₂CH₃, —OCH(CH₃)₂, —SCH₃, —SCH₂CH₃, —SCH₂CH₂CH₃, —NO₂, —NH₂, —F, —Cl, —Br or —I,
   or a pharmaceutically acceptable salt thereof, in an amount effective to ameliorate a locomotor deficit in the subject.

In some embodiments, the method comprises administering to the subject a compound having the structure:

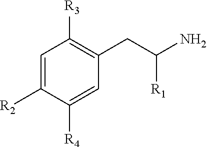

wherein
R₁ is —(C₃-C₁₂ alkyl), -(alkenyl), -(alkynyl), -(cycloalkyl)
R₂ is H, halogen, —NO₂, —CN, —CF₃, —CF₂H, —CH₂OCH₃, —CF₂CH₃, —CF₂OCH₃, -(alkyl), -(alkenyl), -(alkynyl), -(cycloalkyl), -(cycloalkylalkyl), -(heteroalkyl), -(heterocycle), -(heterocycloalkyl), -(aryl), -(heteroaryl), -(hydroxyalkyl), -(haloalkyl), -(alkylaryl), —OH, —O-(alkyl), —O-(alkenyl), —O-(alkynyl), —O-(haloalkyl), —O-(aryl), —O-(heteroaryl), —OCF₃, SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —N-(alkyl)₂, NH-(aryl), —NH-(heteroaryl), —CO₂H, —CO₂-(alkyl), —C(O)—NH₂, —C(O)—NH-(alkyl), —C(O)—NH-(aryl), —SO₂CH₃ or —Si(CH₃)₃;
R₃ is —OCH₃, —OCH₂CH₃, —F or —Cl; and
R₄ is —OCH₃, —OCH₂CH₃ or —SCH₃,
   or a pharmaceutically acceptable salt thereof, in an amount effective to ameliorate a locomotor deficit in the subject.

In some embodiments, a movement disorder comprises Parkinson's disease.

In some embodiments, the movement disorder comprises catatonia.

In some embodiments, the subject is afflicted with dementia, schizophrenia, and/or bipolar disorder.

In some embodiments, the subject is afflicted with anxiety disorder.

In some embodiments, the locomotor deficit comprises a deficit in fine motor skills.

In some embodiments, the locomotor deficit comprises a deficit in balance.

In some embodiments, the locomotor deficit comprises ataxia.

In some embodiments, the amount of the compound is effective to reverse the locomotor deficit.

In some embodiments, a method comprising administering to the subject a compound having the structure wherein
R₁ is —(C₂-C₁₂ alkyl), -(alkenyl), -(alkynyl), -(cycloalkyl); R₂ is H, halogen, —NO₂, —CN, —CF₃, —CF₂H, -(alkyl), -(alkenyl), -(alkynyl), -(cycloalkyl), -(cycloalkylalkyl), -(heteroalkyl), -(heterocycle), -(heterocycloalkyl), -(aryl), -(heteroaryl), -(hydroxyalkyl), -(haloalkyl), -(alkylaryl), —OH, —O-(alkyl), —O-(alkenyl), —O-(alkynyl), —O-(haloalkyl), —O-(aryl), —O-(heteroaryl), —OCF₃, SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —N-(alkyl)₂, NH-(aryl), —NH-(heteroaryl), —CO₂H, —CO₂-(alkyl), —C(O)—NH₂, —C(O)—NH-(alkyl), —C(O)—NH-(aryl) or —Si(CH₃)₃;
R₃ is —OCH₃, —F or —Cl; and
R₄ is —OCH₃ or —SCH₃;
wherein when R₁ is —CH₂CH₃, R₃ is —OCH₃, and R₄ is —OCH₃, then R₂ is other than H, —CH₃, —CH₂CH₃, —CH₂CH₂CH₃, —CH(CH₃)₂, —CH₂CH₂CH₂CH₃, —CH₂OH, —CH(OH) CH₃, —OH, —OCH₂CH₃, —OCH₂CH₂CH₃, —OCH(CH₃)₂, —SCH₃, —SCH₂CH₃, —SCH₂CH₂CH₃, —NO₂, —NH₂, —F, —Cl, —Br or —I.

In some embodiments, a method comprising administering to the subject a compound having the structure wherein
R₁ is —(C₃-C₁₂ alkyl), -(alkenyl), -(alkynyl), -(cycloalkyl);
R₂ is H, halogen, —NO₂, —CN, —CF₃, —CF₂H, -(alkyl), -(alkenyl), -(alkynyl), -(cycloalkyl), -(cycloalkylalkyl), -(heteroalkyl), -(heterocycle), -(heterocycloalkyl), -(aryl), -(heteroaryl), -(hydroxyalkyl), -(haloalkyl), -(alkylaryl), —OH, —O-(alkyl), —O-(alkenyl), —O-(alkynyl), —O-(haloalkyl), —O-(aryl), —O-(heteroaryl), —OCF₃, SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —N-(alkyl)₂, NH-(aryl), —NH-(heteroaryl), —CO₂H, —CO₂-(alkyl), —C(O)—NH₂, —C(O)—NH-(alkyl), —C(O)—NH-(aryl) or —Si(CH₃)₃;
R₃ is —OCH₃, —F or —Cl; and
R₄ is —OCH₃ or —SCH₃.

In some embodiments, a method comprising administering to the subject a compound having the structure wherein
R₁ is —(C₂-C₁₂ alkyl), -(alkenyl), -(alkynyl), -(cycloalkyl);
R₂ is H, —CN, —CF₃, —CH₂OCH₃, —CF₂CH₃, —CF₂OCH₃, -(alkyl), -(alkenyl), -(alkynyl), -(cycloalkyl), -(heterocycle), —S-(aryl), —SO₂CH₃ or —Si(CH₃)₃;
R₃ is —OCH₃, —OCH₂CH₃, —F or —Cl; and
R₄ is —OCH₃, —OCH₂CH₃ or —SCH₃.

In some embodiments, a method comprising administering to the subject a compound having the structure wherein
R₁ is —(C₂-C₁₂ alkyl), -(alkenyl), -(alkynyl), -(cycloalkyl);
R₂ is H, —CN, —CF₃, -(alkyl), -(alkenyl), -(alkynyl), -(cycloalkyl), -(heterocycle), —S-(aryl) or —Si(CH₃)₃;
R₃ is —OCH₃, —F or —Cl; and
R₄ is —OCH₃ or —SCH₃.

In some embodiments, a method comprising administering to the subject a compound having the structure wherein
R₁ is —(C₃-C₁₂ alkyl), -(alkenyl), -(alkynyl), -(cycloalkyl);
R₂ is H, —CN, —CF₃, —CH₂OCH₃, —CF₂CH₃, —CF₂OCH₃, -(alkyl), -(alkenyl), -(alkynyl), -(cycloalkyl), -(heterocycle), —S-(aryl), —SO₂CH₃ or —Si(CH₃)₃;
R₃ is —OCH₃, —OCH₂CH₃, —F or —Cl; and
R₄ is —OCH₃, —OCH₂CH₃ or —SCH₃.

In some embodiments, a method comprising administering to the subject a compound having the structure wherein
R₁ is —(C₃-C₁₂ alkyl), -(alkenyl), -(alkynyl), -(cycloalkyl);
R₂ is H, —CN, —CF₃, -(alkyl), -(alkenyl), -(alkynyl), -(cycloalkyl), -(heterocycle), —S-(aryl) or —Si(CH₃)₃;
R₃ is —OCH₃, —F or —Cl; and
R₄ is —OCH₃ or —SCH₃.

In some embodiments, a method comprising administering to the subject a compound having the structure wherein R₁ is —CH₂CH₃, —CH₂CH₂CH₃, —CH₂CH═CH₂ or cyclopropyl.

In some embodiments, a method comprising administering to the subject a compound having the structure wherein R₁ is —CH₂CH₂CH₃, —CH₂CH═CH₂ or cyclopropyl.

In some embodiments, a method comprising administering to the subject a compound having the structure wherein
R₂ is —CN, —CF₃, —CF₂H, —CH₂OCH₃, —CF₂CH₃, —CF₂OCH₃, —CH₃, —CCH, -cyclopropyl, -cyclobutyl, 2-oxetanyl, —S-(phenyl), —SO₂CH₃ or —Si(CH₃)₃.

In some embodiments, a method comprising administering to the subject a compound having the structure wherein
R₂ is —CN, —CF₃, —CH₃, —CCH, -cyclopropyl, -cyclobutyl, 2-oxetanyl, —S-(phenyl) or —Si(CH₃)₃.

In some embodiments, a method comprising administering to the subject a compound having the structure wherein
R₃ is —OCH₃, —F or —Cl.

In some embodiments, a method comprising administering to the subject a compound having the structure wherein R₃ is —OCH₂CH₃.

In some embodiments, a method comprising administering to the subject a compound having the structure wherein R₄ is —OCH₃ or —SCH₃.

In some embodiments, a method comprising administering to the subject a compound having the structure wherein R₄ is —OCH₂CH₃.

In some embodiments, a method comprising administering to the subject a compound having the structure wherein
R₁ is —CH₂CH₃, —CH₂CH₂CH₃, —CH₂CH═CH₂ or cyclopropyl;
R₂ is —CN, —CF₃, —CH₂OCH₃, —CF₂CH₃, —CF₂OCH₃, —CH₃, —CCH, -cyclopropyl, -cyclobutyl, 2-oxetanyl, —S-(phenyl), —SO₂CH₃ or —Si(CH₃)₃;
R₃ is —OCH₃, —OCH₂CH₃, F or Cl, and
R₄ is —OCH₃, —OCH₂CH₃ or —SCH₃.

In some embodiments, a method comprising administering to the subject a compound having the structure wherein
R₁ is —CH₂CH₃, —CH₂CH₂CH₃, —CH₂CH═CH₂ or cyclopropyl;
R₂ is —CN, —CF₃, —CH₃, —CCH, -cyclopropyl, -cyclobutyl, 2-oxetanyl, —S-(phenyl) or —Si(CH₃)₃;
R₃ is —OCH₃, F or Cl, and
R₄ is —OCH₃ or —SCH₃.

In some embodiments, a method comprising administering to the subject a compound having the structure wherein
R₁ is —CH₂CH₂CH₃, —CH₂CH═CH₂ or cyclopropyl;
R₂ is —CN, —CF₃, —CH₂OCH₃, —CF₂CH₃, —CF₂OCH₃, —CH₃, —CCH, -cyclopropyl, -cyclobutyl, 2-oxetanyl, —S-(phenyl), —SO₂CH₃ or —Si(CH₃)₃;
R₃ is —OCH₃, —OCH₂CH₃, F or Cl, and
R₄ is —OCH₃, —OCH₂CH₃, or —SCH₃.

In some embodiments, a method comprising administering to the subject a compound having the structure wherein
R₁ is —CH₂CH₂CH₃, —CH₂CH═CH₂ or cyclopropyl;
R₂ is —CN, —CF₃, —CH₃, —CCH, -cyclopropyl, -cyclobutyl, 2-oxetanyl, —S-(phenyl) or —Si(CH₃)₃;
R₃ is —OCH₃, F or Cl, and
R₄ is —OCH₃ or —SCH₃.

In some embodiments, a method comprising administering to the subject a compound having the structure:

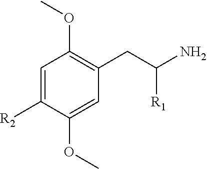

In some embodiments, a method comprising administering to the subject a compound having the structure:

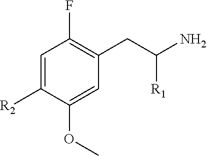

In some embodiments, a method comprising administering to the subject a compound having the structure:

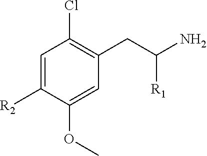

or a pharmaceutically acceptable salt thereof.

In some embodiments, a method comprising administering to the subject a compound having the structure:

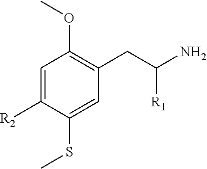

or a pharmaceutically acceptable salt thereof.

In some embodiments, a method comprising administering to the subject a compound having the structure:

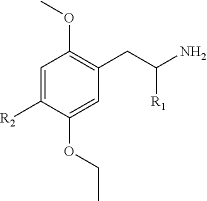

or a pharmaceutically acceptable salt thereof.

In some embodiments, a method comprising administering to the subject a compound having the structure:

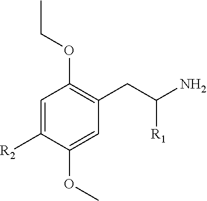

or a pharmaceutically acceptable salt thereof.

In some embodiments, a method comprising administering to the subject a compound having the structure:

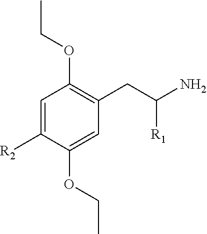

or a pharmaceutically acceptable salt thereof.

In some embodiments, a method comprising administering to the subject a compound having the structure wherein R₁ is —CH₂CH₃, —CH₂CH₂CH₃, —CH₂CH═CH₂ or cyclopropyl.

In some embodiments, a method comprising administering to the subject a compound having the structure wherein R₂ is H, —CN, —CF₃, -(alkyl), -(alkenyl), -(alkynyl), -(cycloalkyl), -(heterocycle), —S-(aryl) or —Si(CH₃)₃.

In some embodiments, a method comprising administering to the subject a compound having the structure wherein R₂ is —CN, —CF₃, —CH₃, —CCH, -cyclopropyl, -cyclobutyl, 2-oxetanyl, —S-(phenyl) or —Si(CH₃)₃.

In some embodiments, a method comprising administering to the subject a compound having the structure wherein R₂ is —CF₃, —CH₂OCH₃ or —CF₂OCH₃.

In some embodiments, a method comprising administering to the subject a compound having the structure wherein R₂ is —CN or —CF₃, or
R₁ is —CH₂CH₃, —CH₂CH₂CH₃, —CH₂CH═CH₂ or cyclopropyl; and
R₂ is —CN or —CF₃, or R₁ is —CH₂CH₃, and R₂ is —CN or —CF₃, or
R₁ is —CH₂CH₂CH₃, and R₂ is —CN or —CF₃, or
R₁ is —CH₂CH═CH₂, and R₂ is —CN or —CF₃, or R₁ is cyclopropyl, and R₂ is —CN or —CF₃.

In some embodiments, a method comprising administering to the subject a compound having the structure wherein R₂ is —CF₃, —CH₂OCH₃, —CF₂CH₃, —CF₂OCH₃ or —SO₂CH₃, or
R₁ is —CH₂CH₃, —CH₂CH₂CH₃, —CH₂CH═CH₂ or cyclopropyl; and
R₂ is —CF₃, —CH₂OCH₃, —CF₂CH₃, —CF₂OCH₃ or —SO₂CH₃, or
R₁ is —CH₂CH₃, and R₂ is CF₃, —CH₂OCH₃, —CF₂CH₃, —CF₂OCH₃ or —SO₂CH₃, or
R₁ is —CH₂CH₂CH₃, and R₂ is CF₃, —CH₂OCH₃, —CF₂CH₃, —CF₂OCH₃ or —SO₂CH₃, or
R₁ is —CH₂CH═CH₂, and R₂ is CF₃, —CH₂OCH₃, —CF₂CH₃, —CF₂OCH₃ or —SO₂CH₃, or
R₁ is cyclopropyl, and R₂ is CF₃, —CH₂OCH₃, —CF₂CH₃, —CF₂OCH₃ or —SO₂CH₃.

In some embodiments, a method comprising administering to the subject a compound having the structure wherein R₁ is —CH₂CH₃; and
R₂ is —CF₃, —CH₂OCH₃ or —CF₂OCH₃.

In some embodiments, a method comprising administering to the subject a compound having the structure:

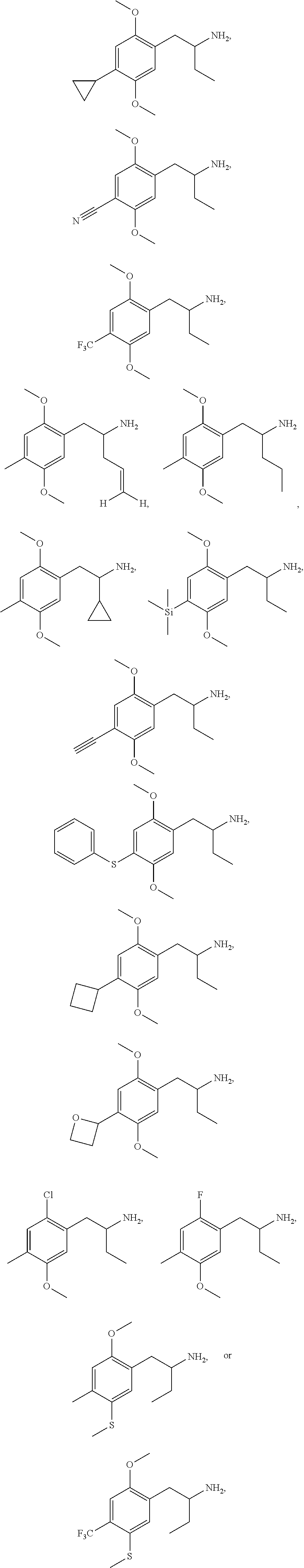

or a pharmaceutically acceptable salt thereof.

In some embodiments, a method comprising administering to the subject a compound having the structure:

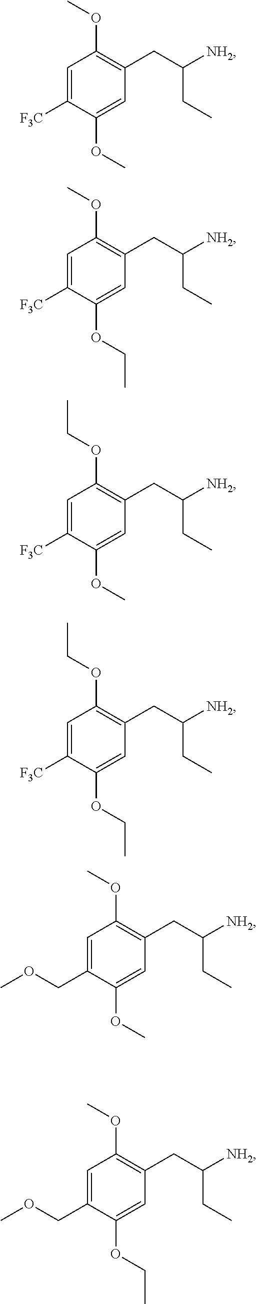

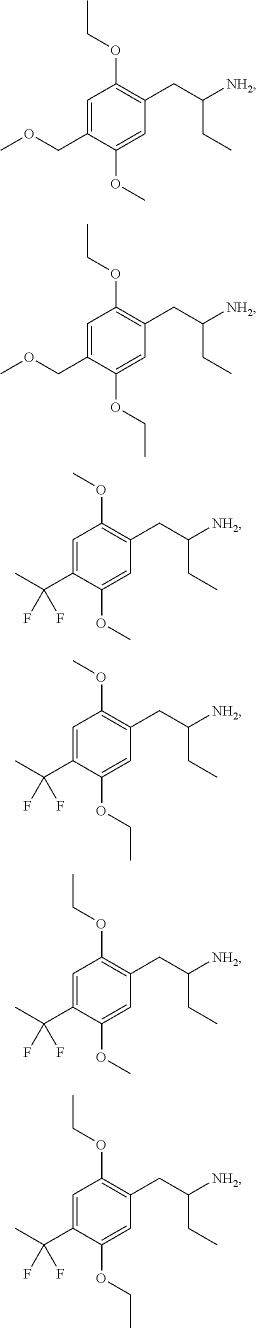

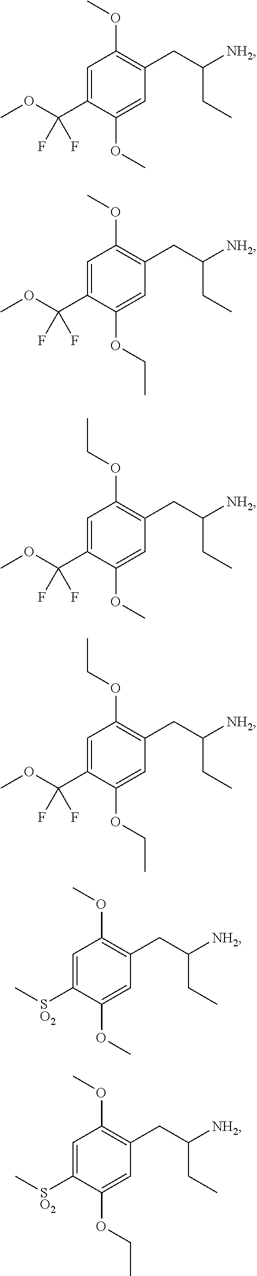

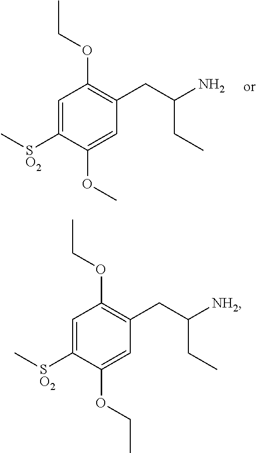

or a pharmaceutically acceptable salt thereof.

In some embodiments, a method comprising administering to the subject a compound having the structure wherein
R₁ is —CH₂CH₃,
R₂ is —CF₃, —CH₂OCH₃ or —CF₂OCH₃,
R₃ is —OCH₃; and
R₄ is —OCH₃.

In some embodiments, a method comprising administering to the subject a compound having the structure:

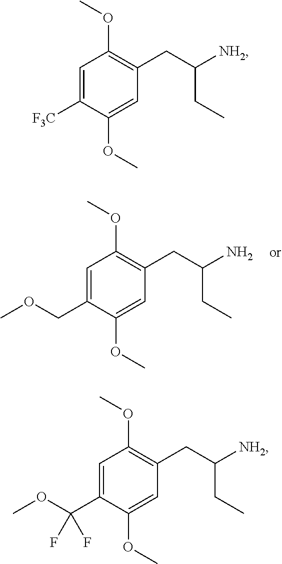

or a pharmaceutically acceptable salt thereof.

In some embodiments, a method comprising administering to the subject a compound having the structure wherein
R₁ is —CH₂CH₃;
R₂ is —CHFCH₃, —CF₂CH₃, —CHFCH₂CH₃, —CH₂CHFCH₃, —CF₂CH₂CH₃, —CH₂CF₂CH₃, —CHFCH₂CH₂CH₃, —CH₂CFHCH₂CH₃, —CH₂CH₂CHFCH₃, —CF₂CH₂CH₂CH₃, —CH₂CF₂CH₂CH₃, or —CH₂CH₂CF₂CH₃;
R₃ is —OCH₃; and
R₄ is —OCH₃.

In some embodiments, a method comprising administering to the subject a compound having the structure:

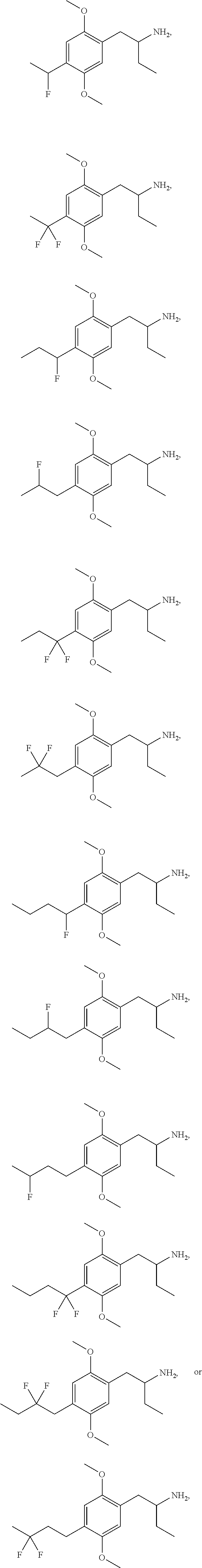

or a pharmaceutically acceptable salt thereof.

In some embodiments, a method comprising administering to the subject a compound having the structure:

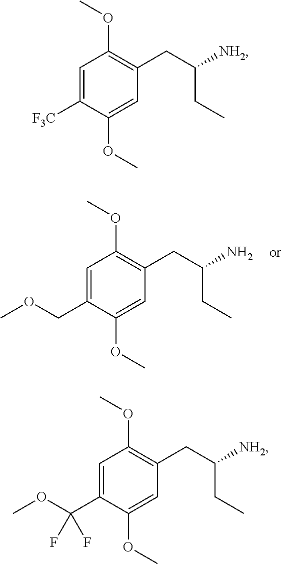

or a pharmaceutically acceptable salt thereof.

In some embodiments, a method comprising administering to the subject a compound in a pharmaceutical composition comprising the compound and a pharmaceutically acceptable carrier.

In some embodiments, a method comprising administering to the subject a compound wherein administering the compound activates the 5HT2A receptor.

In some embodiments, a method comprising administering to the subject a compound wherein the compound selectively activates 5HT2A receptor compared to the 5HT2B receptor.

In some embodiments, a method comprising administering to the subject a compound wherein the subject is afflicted with dementia, Alzheimer's disease, attention deficit hyperactivity disorder (ADHD), schizophrenia, depression, bipolar disorder, anxiety disorder, obsessive-compulsive disorder (OCD), stress disorder, a substance use disorder, opioid withdrawal symptoms, cluster headache, diabetic retinopathy, dry eyes, macular degeneration or glaucoma.

In some embodiments, a method comprising administering to the subject a compound wherein alertness and or ability to learn in the subject is enhanced.

In some embodiments, a method comprising administering to the subject a compound wherein the effective amount of the compound administered to the subject does not induce a stimulant effect, or wherein the effective amount of the compound administered to the subject does not induce a hallucinogenic effect, or wherein the effective amount of the compound administered to the subject does not induce a stimulant effect and a hallucinogenic effect.

In some embodiments, a method comprising administering to the subject a compound wherein the subject is a mammal.

In some embodiments, a method comprising administering to the subject a compound wherein the mammal is a human.

In some embodiments, a method comprising administering to the subject a compound wherein the effective amount is between about 25 and about 500 mg of the compound.

In some embodiments, a method comprising administering to the subject a compound wherein the effective amount is between about 100 and 300 mg of the compound.

In some embodiments, a method comprising administering to the subject a compound wherein the effective amount is between about 0.1-20 mg/kg of compound per kilogram of body weight.

In some embodiments, a method comprising administering to the subject a compound wherein the compound has the structure:

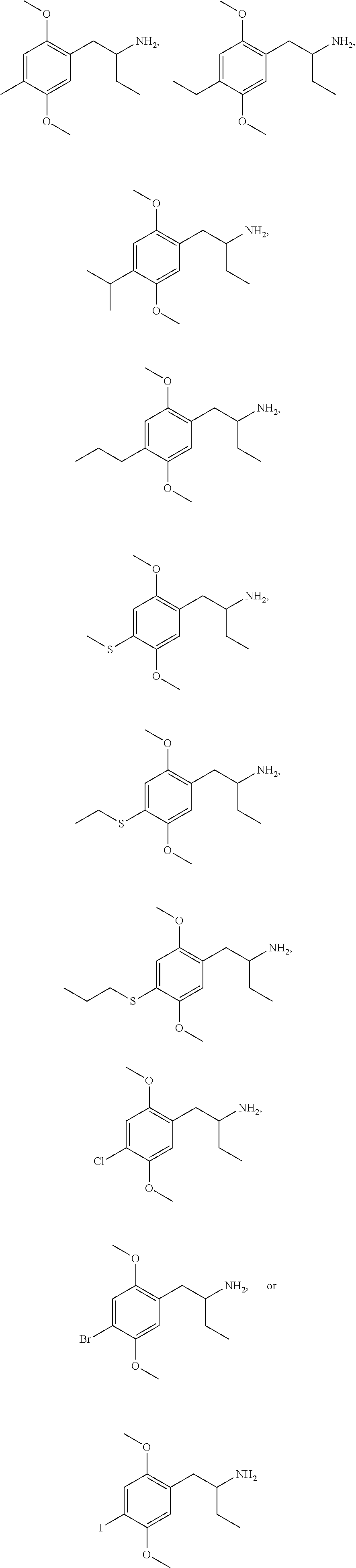

or a pharmaceutically acceptable salt thereof.

In some embodiments, a method comprising administering to the subject a compound wherein the compound has the structure:

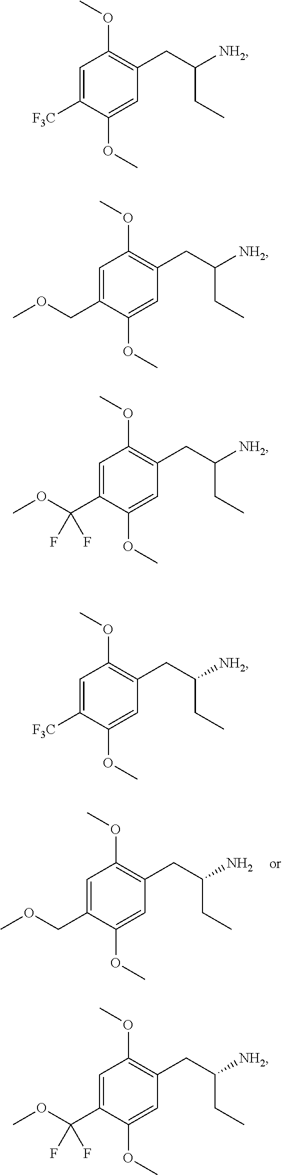

or a pharmaceutically acceptable salt thereof.

In some embodiments, a method comprising administering to the subject a compound having the structure:

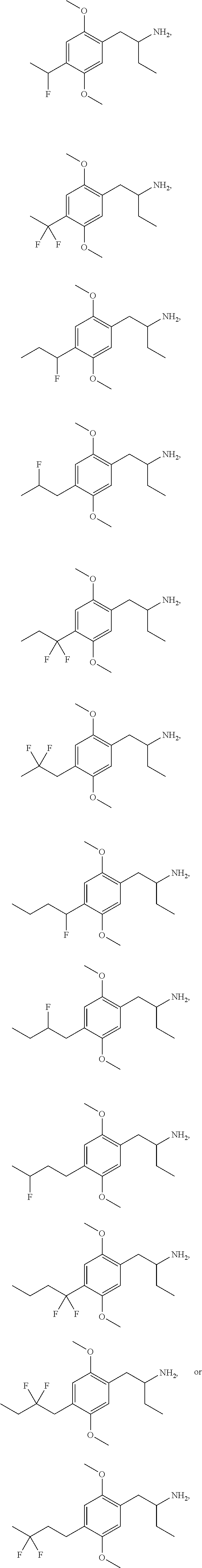

or a pharmaceutically acceptable salt thereof.

In some embodiments, a method comprising administering to the subject a compound wherein the compound has the structure:

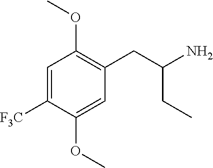 [SMILES: CCC(N)Cc1cc(OC)c(C(F)(F)F)cc1OC]

or a pharmaceutically acceptable salt thereof.

In some embodiments, a method comprising administering to the subject a compound wherein the compound has the structure:

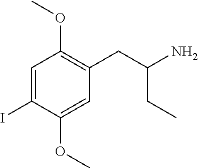 [SMILES: CCC(N)Cc1cc(OC)c(I)cc1OC]

or a pharmaceutically acceptable salt thereof.

In some embodiments, a method comprising administering to the subject a compound wherein the compound has the structure:

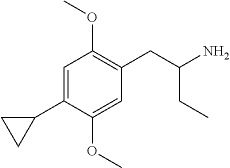 [SMILES: CCC(N)Cc1cc(OC)c(C2CC2)cc1OC]

or a pharmaceutically acceptable salt thereof.

In some embodiments, a method comprising administering to the subject a compound wherein the compound has the structure:

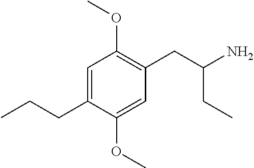 [SMILES: CCCc1cc(OC)c(CC(N)CC)cc1OC]

or a pharmaceutically acceptable salt thereof.

In some embodiments, a method comprising administering to the subject a compound wherein the compound has the structure:

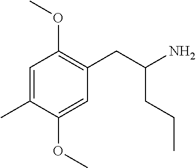 [SMILES: CCCC(N)Cc1cc(OC)c(C)cc1OC]

or a pharmaceutically acceptable salt thereof.

In some embodiments, a method comprising administering to the subject a compound wherein the compound has the structure:

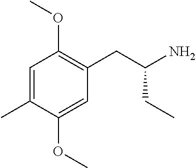 [SMILES: CC[C@@H](N)Cc1cc(OC)c(C)cc1OC]

or a pharmaceutically acceptable salt thereof.

In some embodiments, a method comprising administering to the subject a compound wherein the compound has the structure:

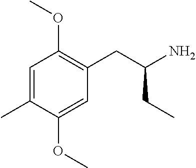 [SMILES: CC[C@H](N)Cc1cc(OC)c(C)cc1OC]

or a pharmaceutically acceptable salt thereof.

The present invention also provides a pharmaceutical composition comprising the compound of the present application and a pharmaceutically acceptable carrier.

The present invention also provides a method of activating 5HT2A receptor comprising contacting the 5HT2A receptor with the compound of the present application.

The present invention also provides a method of selectively activating 5HT2A receptor compared to the 5HT2B receptor, comprising selectively contacting the 5HT2A receptor compared to the 5HT2B receptor with the compound of the present application.

The present invention also provides a method of treating a subject afflicted with Parkinson's disease comprising administering an effective amount of the compound of the present application or the composition of the present application to the subject so as to treat the Parkinson's disease.

The present invention also provides a method of treating a subject afflicted with dementia or Alzheimer's disease comprising administering an effective amount of the compound of the present application or the composition of the present application to the subject so as to treat the dementia or Alzheimer's disease.

The present invention also provides a method of treating a subject afflicted with attention deficit hyperactivity disorder (ADHD) comprising administering an effective amount of the compound of the present application or the composition of the present application to the subject so as to treat the ADHD.

The present invention also provides a method of treating a subject afflicted with schizophrenia comprising administering an effective amount of the compound of the present application or the composition of the present application to the subject so as to treat the schizophrenia.

The present invention also provides a method of treating a subject afflicted with depression, bipolar disorder, anxiety disorder, obsessive-compulsive disorder (OCD) or stress disorder comprising administering an effective amount of the compound of the present application or the composition of the present application to the subject so as to treat the depression, bipolar disorder, anxiety disorder, obsessive-compulsive disorder (OCD) or stress disorder.

The present invention also provides a method of treating a subject afflicted with a substance use disorder comprising administering an effective amount of the compound of the present application or the composition of the present application to the subject so as to treat the substance use disorder.

In some embodiments of the above methods, wherein the substance use disorder is opioid use disorder, alcohol use disorder or stimulant use disorder including nicotine use disorder.

In some embodiments of the above methods, wherein the substance is an opioid.

In some embodiments of any of the above methods, wherein the opioid is morphine, hydromorphone, oxymorphone, codeine, dihydrocodeine, hydrocodone, oxycodone, nalbuphine, butorphanol, etorphine, dihydroetorphine, levorphanol, metazocine, pentazocine, meptazinol, meperidine (pethidine), buprenorphine, methadone, tramadol, tapentadol, mitragynine, 3-deutero-mitragynine, 7-hydroxymitragynine, 3-deutero-7-hydroxymitragynine, mitragynine pseudoindoxyl or tianeptine.

In some embodiments of any of the above methods, wherein the opioid is fentanyl, sufentanil, alfentanil, furanylfentanyl, 3-methylfentanyl, valerylfentanyl, butyrylfentanyl, β-Hydroxythiofentanyl, acrylfentanyl or carfentanil.

In some embodiments of any of the above methods, wherein the stimulant is cocaine, amphetamine, methamphetamine or cathinone and its derivatives.

In some embodiments of any of the above methods, wherein the stimulant is nicotine.

The present invention also provides a method of treating a subject afflicted with opioid withdrawal symptoms comprising administering an effective amount of the compound of the present application or the composition of the present application to the subject so as to treat the opioid withdrawal symptoms.

In some embodiments of the above methods, wherein a symptom of substance use disorder is opioid withdrawal or mitigation of relapse to opioid use or SUD.

In some embodiments of the above methods, wherein the risk of relapse to the use of opioids, alcohol or stimulants is reduced.

In some embodiments of the above methods, wherein self-administration of an opioid, alcohol or stimulant is reduced.

The present invention also provides a method of treating a subject afflicted with cluster headache comprising administering an effective amount of the compound of the present application or the composition of the present application to the subject so as to treat the cluster headache.

The present invention also provides a method of treating a subject afflicted with diabetic retinopathy, dry eyes, macular degeneration or glaucoma comprising administering an effective amount of the compound of the present application or the composition of the present application to the subject so as to treat the diabetic retinopathy, dry eyes, macular degeneration or glaucoma.

The present invention also provides a method of treating a subject afflicted with catatonia, comprising administering an effective amount of the compound of the present application or the composition of the present application to the subject so as to treat the catatonia.

The present invention also provides a method of enhancing alertness in a subject, comprising administering an effective amount of the compound of the present application or the composition of the present application to the subject so as to enhance alertness.

The present invention also provides a method of enhancing ability to learn in a subject, comprising administering an effective amount of the compound of the present application or the composition of the present application to the subject so as to enhance ability to learn.

In some embodiments of any of the above methods, wherein the effective amount of the compound administered to the subject does not induce a stimulant effect.

In some embodiments of any of the above methods, wherein the effective amount of the compound administered to the subject does not induce a hallucinogenic effect.

In some embodiments of any of the above methods, wherein the effective amount of the compound administered to the subject does not induce a stimulant effect and a hallucinogenic effect.

In some embodiments of any of the above methods, wherein the subject is a mammal.

In some embodiments of the above methods, wherein the mammal is a human.

In some embodiments of any of the above methods, wherein the effective amount of 10-500 mg of the compound is administered to the subject.

The present invention provides a method of activating 5HT2A receptor in a subject comprising administering to a subject an effective amount of a compound having the structure:

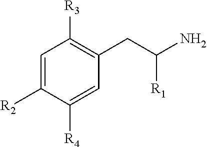

wherein
   R₁ is —(C₂-C₁₂ alkyl), -(alkenyl), -(alkynyl), -(cycloalkyl)
   R₂ is H, halogen, —NO₂, —CN, —CF₃, —CF₂H, —CH₂OCH₃, —CF₂CH₃, —CF₂OCH₃, -(alkyl), -(alkenyl), -(alkynyl), -(cycloalkyl), -(cycloalkylalkyl), -(heteroalkyl), -(heterocycle), -(heterocycloalkyl), -(aryl), -(heteroaryl), -(hydroxyalkyl), -(haloalkyl), -(alkylaryl), —OH, —O-(alkyl), —O-(alkenyl), —O-(alkynyl), —O-(haloalkyl), —O-(aryl), —O-(heteroaryl), —OCF₃, SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —N-(alkyl)₂, NH-(aryl), —NH-(heteroaryl), —CO₂H, —CO₂-(alkyl), —C(O)—NH₂, —C(O)—NH-(alkyl), —C(O)—NH-(aryl), —SO₂CH₃ or —Si(CH₃)₃;
      R₃ is —OCH₃, —OCH₂CH₃, —F or —Cl; and
      R₄ is —OCH₃, —OCH₂CH₃, —SCH₃, —F or —Cl,
         or a pharmaceutically acceptable salt thereof, so as to thereby activate 5HT2A receptor in a subject.

The present invention also provides a method of activating 5HT2A receptor in a subject comprising administering to a subject an effective amount of a compound having the structure:

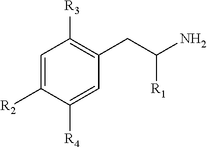

wherein
R₁ is —(C₂-C₁₂ alkyl), -(alkenyl), -(alkynyl), -(cycloalkyl)
R₂ is H, halogen, —NO₂, —CN, —CF₃, —CF₂H, -(alkyl), -(alkenyl), -(alkynyl), -(cycloalkyl), -(cycloalkylalkyl), -(heteroalkyl), -(heterocycle), -(heterocycloalkyl), -(aryl), -(heteroaryl), -(hydroxyalkyl), -(haloalkyl), -(alkylaryl), —OH, —O-(alkyl), —O-(alkenyl), —O-(alkynyl), —O-(haloalkyl), —O-(aryl), —O-(heteroaryl), —OCF₃, SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —N-(alkyl)₂, NH-(aryl), —NH-(heteroaryl), —CO₂H, —CO₂-(alkyl), —C(O)—NH₂, —C(O)—NH-(alkyl), —C(O)—NH-(aryl) or —Si(CH₃)₃;
R₃ is —OCH₃, —F or —Cl; and
R₄ is —OCH₃, —SCH₃, —F or —Cl;
   or a pharmaceutically acceptable salt thereof, so as to thereby activate 5HT2A receptor in a subject.

The present invention provides a method of treating a subject afflicted with Parkinson's disease by activating 5HT2A receptor in a subject.

The present invention provides a method of treating a subject afflicted with dementia or Alzheimer's disease by activating 5HT2A receptor in a subject.

The present invention provides a method of treating a subject afflicted with attention deficit hyperactivity disorder (ADHD) by activating 5HT2A receptor in a subject.

The present invention provides a method of treating a subject afflicted with schizophrenia by activating 5HT2A receptor in a subject.

The present invention provides a method of treating a subject afflicted with depression, bipolar disorder, anxiety disorder, obsessive-compulsive disorder (OCD) or stress disorder by activating 5HT2A receptor in a subject.

The present invention provides a method of treating a subject afflicted with a substance use disorder by activating 5HT2A receptor in a subject.

The present invention provides a method of treating a subject afflicted with opioid use disorder by activating 5HT2A receptor in a subject.

The present invention provides a method of treating a subject afflicted with alcohol use disorder by activating 5HT2A receptor in a subject.

The present invention provides a method of treating a subject afflicted with stimulant use disorder by activating 5HT2A receptor in a subject.

The present invention provides a method of treating a subject afflicted with nicotine use disorder by activating 5HT2A receptor in a subject.

In some embodiments of any of the above methods, wherein the substance is an opioid.

In some embodiments of any of the above methods, wherein the opioid is morphine, hydromorphone, oxymorphone, codeine, dihydrocodeine, hydrocodone, oxycodone, nalbuphine, butorphanol, etorphine, dihydroetorphine, levorphanol, metazocine, pentazocine, meptazinol, meperidine (pethidine), buprenorphine, methadone, tramadol, tapentadol, mitragynine, 3-deutero-mitragynine, 7-hydroxymitragynine, 3-deutero-7-hydroxymitragynine, mitragynine pseudoindoxyl or tianeptine.

In some embodiments of any of the above methods, wherein the opioid is fentanyl, sufentanil, alfentanil, furanylfentanyl, 3-methylfentanyl, valerylfentanyl, butyrylfentanyl, β-Hydroxythiofentanyl, acrylfentanyl or carfentanil.

In some embodiments of any of the above methods, wherein the stimulant is cocaine, amphetamine, methamphetamine or cathinone and its derivatives.

In some embodiments of any of the above methods, wherein the stimulant is nicotine.

The present invention provides a method of treating a subject afflicted with opioid withdrawal symptoms by activating 5HT2A receptor in a subject.

In some embodiments of any of the above methods, wherein a symptom of substance use disorder is opioid withdrawal or mitigation of relapse to opioid use or SUD.

In some embodiments of any of the above methods, wherein the risk of relapse to the use of opioids, alcohol or stimulants is reduced.

In some embodiments of any of the above methods, wherein self-administration of an opioid, alcohol or stimulant is reduced.

The present invention provides a method of treating a subject afflicted with cluster headache by activating 5HT2A receptor in a subject.

The present invention provides a method of treating a subject afflicted with diabetic retinopathy, dry eyes, macular degeneration or glaucoma by activating 5HT2A receptor in a subject.

In some embodiments of any of the above methods, wherein the effective amount of the compound administered to the subject does not induce a stimulant effect.

In some embodiments of any of the above methods, wherein the effective amount of the compound administered to the subject does not induce a hallucinogenic effect.

In some embodiments of any of the above methods, wherein the effective amount of the compound administered to the subject does not induce a stimulant effect and a hallucinogenic effect.

In some embodiments of any of the above methods, wherein the subject is a mammal.

In some embodiments of the above methods, wherein the mammal is a human.

In some embodiments of any of the above methods, wherein an effective amount of 10-500 mg of the compound is administered to the subject.

The present invention also provides a compound having the structure:

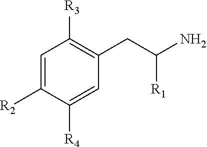

wherein
R₁ is —(C₂-C₁₂ alkyl), -(alkenyl), -(alkynyl), -(cycloalkyl);
R₂ is H, halogen, —NO₂, —CN, —CF₃, —CF₂H, —CH₂OCH₃, —CF₂CH₃, —CF₂OCH₃, -(alkyl), -(alkenyl), -(alkynyl), -(cycloalkyl), -(cycloalkylalkyl), -(heteroalkyl), -(heterocycle), -(heterocycloalkyl), -(aryl), -(heteroaryl), -(hydroxyalkyl), -(haloalkyl), -(alkylaryl), —OH, —O-(alkyl), —O-(alkenyl), —O-(alkynyl), —O-(haloalkyl), —O-(aryl), —O-(heteroaryl), —OCF₃, SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —N-(alkyl)₂, NH-(aryl), —NH-(heteroaryl), —CO₂H, —CO₂-(alkyl), —C(O)—NH₂, —C(O)—NH-(alkyl), —C(O)—NH-(aryl), —SO₂CH₃ or —Si(CH₃)₃;
R₃ is —OCH₃, —OCH₂CH₃, —F or —Cl; and
R₄ is —OCH₃, —OCH₂CH₃ or —SCH₃;
wherein when R₁ is —CH₂CH₃, R₃ is —OCH₃, and R₄ is —OCH₃, then R₂ is other than H, —CH₃, —CH₂CH₃, —CH₂CH₂CH₃, —CH(CH₃)₂, —CH₂CH₂CH₂CH₃, —CH₂OH, —CH(OH) CH₃, —OH, —OCH₂CH₃, —OCH₂CH₂CH₃, —OCH(CH₃)₂, —SCH₃, —SCH₂CH₃, —SCH₂CH₂CH₃, —NO₂, —NH₂, —F, —Cl, —Br or —I,
or a pharmaceutically acceptable salt thereof.

In other embodiments, the compound is as described herein.

In some embodiments, a pharmaceutical composition comprising a compound of the present invention and a pharmaceutically acceptable carrier.

In some embodiments of the above methods, the compound has the structure:

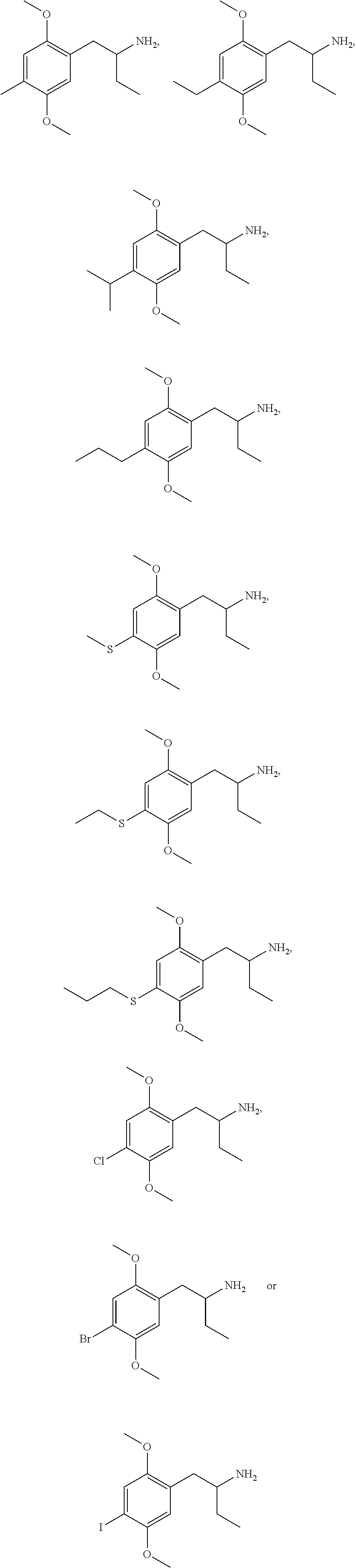

or a pharmaceutically acceptable salt thereof.

In some embodiments of the above methods, the compound has the structure:

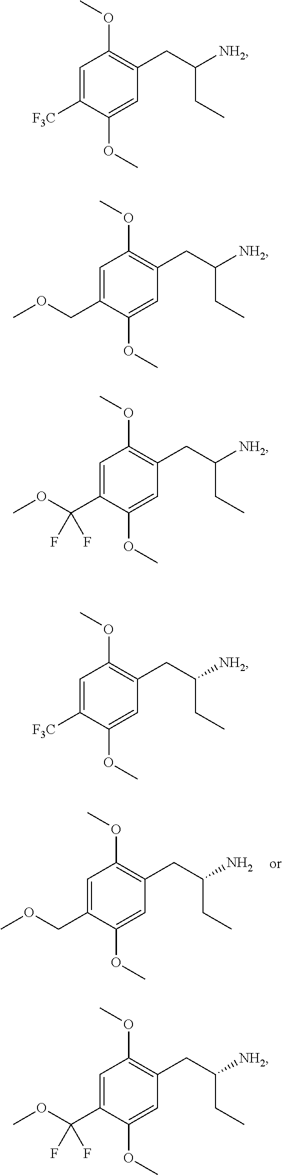

or a pharmaceutically acceptable salt thereof.

In some embodiments of the above methods, the compound has the structure:

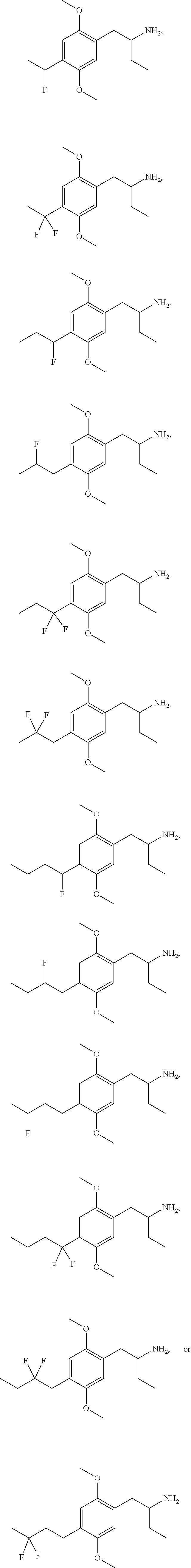

or a pharmaceutically acceptable salt thereof.

The terms “movement disorder,” “neurological movement disorder” or “neurological condition,” as used herein, within are well the understanding of one of ordinary skill in the art and are used broadly and to refer to any brain disease, anomaly, or condition causing a subject to have abnormal voluntary and/or involuntary movements, or slow, reduced movements. Examples of movement disorders include, but are not limited to, Parkinson's disease, dystonia, Huntington's disease, essential tremor, anxiety, chorea, myoclonus, ballismus, dysmetria, postural disorders, spasticity (e.g. focal spasticity from stroke, upper limb spasticity), blepharospasm, multiple sclerosis, cerebral palsy, mood disorders, sleep disorders, obesity, anorexia, and chronic pain disorders.

Opioid use disorder (OUD) involves, but is not limited to, misuse of opioid medications or use of illicitly obtained opioids. The Diagnostic and Statistical Manual of Mental Disorders, 5^{th} Edition (American Psychiatric Association: Diagnostic and Statistical Manual of Mental Disorders: Diagnostic and Statistical Manual of Mental Disorders, Fifth Edition. Arlington, VA: American Psychiatric Association, 2013), which is hereby incorporated by reference, describes opioid use disorder as a problematic pattern of opioid use leading to problems or distress, with at least two of the following occurring within a 12-month period:
Taking larger amounts or taking drugs over a longer period than intended.
Persistent desire or unsuccessful efforts to cut down or control opioid use.
Spending a great deal of time obtaining or using the opioid or recovering from its effects.
Craving, or a strong desire or urge to use opioids.
Problems fulfilling obligations at work, school, or home.
Continued opioid use despite having recurring social or interpersonal problems.
Giving up or reducing activities because of opioid use.
Using opioids in physically hazardous situations.
Continued opioid use despite ongoing physical or psychological problem likely to have been caused or worsened by opioids.
Tolerance (i.e., need for increased amounts or diminished effect with continued use of the same amount).
Experiencing withdrawal (opioid withdrawal syndrome) or taking opioids (or a closely related substance) to relieve or avoid withdrawal symptoms.

Alcohol use disorder (AUD) involves, but is not limited to, a chronic relapsing brain disease characterized by compulsive alcohol use, loss of control over alcohol intake, and a negative emotional state when not using. The Diagnostic and Statistical Manual of Mental Disorders, 5^{th} Edition describes alcohol use disorder as a problematic pattern of alcohol use leading to problems or distress, with at least two of the following occurring within a 12-month period:
Being unable to limit the amount of alcohol you drink.
Wanting to cut down on how much you drink or making unsuccessful attempts to do so.
Spending a lot of time drinking, getting alcohol, or recovering from alcohol use.
Feeling a strong craving or urge to drink alcohol.
Failing to fulfill major obligations at work, school or home due to repeated alcohol use.
Continuing to drink alcohol even though you know it is causing physical, social, or interpersonal problems.
Giving up or reducing social and work activities and hobbies.
Using alcohol in situations where it is not safe, such as when driving or swimming.
Developing a tolerance to alcohol so you need more to feel its effect, or you have a reduced effect from the same amount.
Experiencing withdrawal symptoms—such as nausea, sweating and shaking—when you do not drink, or drinking to avoid these symptoms.

Stimulant use disorder involves, but is not limited to, a pattern of problematic use of amphetamine, methamphetamine, cocaine, or other stimulants except caffeine or nicotine, leading to at least two of the following problems within a 12-month period:
Taking more stimulants than intended.
Unsuccessful in trying to cut down or control use of stimulants, despite wanting to do so.
Spending excessive amounts of time to activities surrounding stimulant use.
Urges and cravings for stimulants.
Failing in the obligations of home, school, or work.
Carrying on taking stimulants, even though it has led to relationship or social problems.
Giving up or reducing important recreational, social, or work-related activities because of using stimulants.
Using stimulants in a physically hazardous way.
Continuing to use stimulants even while knowing that it is causing or worsening a physical or psychological problem.
Tolerance to stimulants.
Withdrawal from stimulants if you do not take them.

Polydrug use disorder or polysubstance use disorder involves, but is not limited to, dependence on multiple drugs or substances.

The term “5HT2A” refers to the serotonin 2A receptor.

The term “5HT2B” refers to the serotonin 2B receptor.

The term “5HT2A agonist” or “5HT2A receptor agonist” is intended to mean any compound or substance that activates the 5HT2A receptor. The agonist may be a partial, full, super, or biased agonist.

The term “non-hallucinogenic psychedelics” is intended to mean 5HT2A receptor agonists that induce no or limited hallucinogenic effects at pharmacologically and physiologically meaningful engagement of the target receptor.

The term “PTSD” refers to post-traumatic stress disorder.

The term “OCD” refers to obsessive-compulsive disorder.

The term “ADHD” refers to attention deficit hyperactivity disorder.

The term “LSD” refers to lysergic acid diethylamide.

Except where otherwise specified, the structure of a compound of this invention includes an asymmetric carbon atom, it is understood that the compound occurs as a racemate, racemic mixture, and isolated single enantiomer. All such isomeric forms of these compounds are expressly included in this invention. Except where otherwise specified, each stereogenic carbon may be of the R or S configuration. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis, such as those described in “Enantiomers, Racemates and Resolutions” by J. Jacques, A. Collet and S. Wilen, Pub. John Wiley & Sons, NY, 1981. For example, the resolution may be carried out by preparative chromatography on a chiral column.

The subject invention is also intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notation of a carbon in structures throughout this application, when used without further notation, are intended to represent all isotopes of carbon, such as ¹²C, ¹³C, or ¹⁴C. Furthermore, any compounds containing ¹³C or ¹⁴C may specifically have the structure of any of the compounds disclosed herein.

It will also be noted that any notation of a hydrogen in structures throughout this application, when used without further notation, are intended to represent all isotopes of hydrogen, such as ¹H, ²H, or ³H. Furthermore, any compounds containing ²H or ³H may specifically have the structure of any of the compounds disclosed herein.

Isotopically-labeled compounds can generally be prepared by conventional techniques known to those skilled in the art using appropriate isotopically-labeled reagents in place of the non-labeled reagents employed.

In the compounds used in the method of the present invention, the substituents may be substituted or unsubstituted, unless specifically defined otherwise.

In the compounds used in the method of the present invention, alkyl, heteroalkyl, monocycle, bicycle, aryl, heteroaryl and heterocycle groups can be further substituted by replacing one or more hydrogen atoms with alternative non-hydrogen groups. These include, but are not limited to, halo, hydroxy, mercapto, amino, carboxy, cyano and carbamoyl.

It is understood that substituents and substitution patterns on the compounds used in the method of the present invention can be selected by one of ordinary skill in the art to provide compounds that are chemically stable and that can be readily synthesized by techniques known in the art from readily available starting materials. If a substituent is itself substituted with more than one group, it is understood that these multiple groups may be on the same carbon or on different carbons, so long as a stable structure results.

In choosing the compounds used in the method of the present invention, one of ordinary skill in the art will recognize that the various substituents, i.e. R₁, R₂, etc. are to be chosen in conformity with well-known principles of chemical structure connectivity.

As used herein, “alkyl” is intended to include both branched and straight-chain saturated aliphatic hydrocarbon groups having the specified number of carbon atoms. Thus, C₁-Cₙ as in “C₁-Cₙ alkyl” is defined to include groups having 1, 2 . . . , n−1 or n carbons in a linear or branched arrangement, and specifically includes methyl, ethyl, propyl, butyl, pentyl, hexyl, heptyl, isopropyl, isobutyl, sec-butyl and so on. An embodiment can be C₁-C₁₂ alkyl, C₂-C₁₂ alkyl, C₃-C₁₂ alkyl, C₄-C₁₂ alkyl and so on. An embodiment can be C₁-C₈ alkyl, C₂-C₈ alkyl, C₃-C₈ alkyl, C₄-Cₑ alkyl and so on. “Alkoxy” represents an alkyl group as described above attached through an oxygen bridge.

The term “alkenyl” refers to a non-aromatic hydrocarbon radical, straight or branched, containing at least 1 carbon to carbon-to-carbon double bond, and up to the maximum possible number of non-aromatic carbon-carbon double bonds may be present. Thus, C₂-Cₙ alkenyl is defined to include groups having 1, 2 . . . , n−1 or n carbons. For example, “C₂-C₆ alkenyl” means an alkenyl radical having 2, 3, 4, 5, or 6 carbon atoms, and at least 1 carbon-carbon double bond, and up to, for example, 3 carbon-carbon double bonds in the case of a C₆ alkenyl, respectively. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated. An embodiment can be C₂-C₁₂ alkenyl or C₂-C₈ alkenyl.

The term “alkynyl” refers to a hydrocarbon radical straight or branched, containing at least 1 carbon-to-carbon triple bond, and up to the maximum possible number of non-aromatic carbon-carbon triple bonds may be present. Thus, C₂-Cₙ alkynyl is defined to include groups having 1, 2 . . . , n−1 or n carbons. For example, “C₂-C₆ alkynyl” means an alkynyl radical having 2 or 3 carbon atoms, and 1 carbon-carbon triple bond, or having 4 or 5 carbon atoms, and up to 2 carbon-carbon triple bonds, or having 6 carbon atoms, and up to 3 carbon-carbon triple bonds. Alkynyl groups include ethynyl, propynyl and butynyl. As described above with respect to alkyl, the straight or branched portion of the alkynyl group may contain triple bonds and may be substituted if a substituted alkynyl group is indicated. An embodiment can be a C₂-Cₙ alkynyl. An embodiment can be C₂-C₁₂ alkynyl or C₃-C₈ alkynyl.

As used herein, “hydroxyalkyl” includes alkyl groups as described above wherein one or more bonds to hydrogen contained therein are replaced by a bond to an —OH group. In some embodiments, C₁-C₁₂ hydroxyalkyl or C₁-C₆ hydroxyalkyl. C₁-Cₙ as in “C₁-Cₙ alkyl” is defined to include groups having 1, 2, . . . , n−1 or n carbons in a linear or branched arrangement (e.g. C₁-C₂ hydroxyalkyl, C₁-C₃ hydroxyalkyl, C₁-C₄ hydroxyalkyl, C₁-C₅ hydroxyalkyl, or C₁-C₆ hydroxyalkyl) For example, C₁-C₆, as in “C₁-C₆ hydroxyalkyl” is defined to include groups having 1, 2, 3, 4, 5, or 6 carbons in a linear or branched alkyl arrangement wherein a hydrogen contained therein is replaced by a bond to an —OH group.

As used herein, “heteroalkyl” includes both branched and straight-chain saturated aliphatic hydrocarbon groups having the specified number of carbon atoms and at least 1 heteroatom within the chain or branch.

As used herein, “haloalkyl” includes alkyl groups as described above wherein one or more bonds to hydrogen contained therein are replaced by a bond to a —X group, where X is a halogen atom. C₁-Cₙ as in “C₁-Cₙ alkyl” is defined to include groups having 1, 2, . . . , n−1 or n carbons in a linear or branched arrangement (e.g. C₁-C₂ haloalkyl, C₁-C₃ haloalkyl, C₁-C₄ haloalkyl, C₁-C₅ haloalkyl, or C₁-C₆ haloalkyl) For example, C₁-C₆, as in “C₁-C₆ haloalkyl” is defined to include groups having 1, 2, 3, 4, 5, or 6 carbons in a linear or branched alkyl arrangement wherein one or more hydrogen atoms contained therein is replaced by a bond to an —X group.

In some embodiments, the haloalkyl is fluoroalkyl.

In some embodiments, the haloalkyl is C₁-C₁₂ fluoroalkyl or C₁-C₆ fluoroalkyl.

In some embodiments, the fluoroalkyl is —CF₃ or —CH₂F.

In some embodiments, the fluoroalkyl is —CH₂F, —CHF₂, —CF₃, —CHFCH₃, —CF₂CH₃, —CHFCH₂CH₃, —CH₂CHFCH₃, —CF₂CH₂CH₃, —CH₂CF₂CH₃, —CHFCH₂CH₂CH₃, —CH₂CHFCH₂CH₃, —CH₂CH₂CHFCH₃, —CF₂CH₂CH₂CH₃, —CH₂CF₂CH₂CH₃, —CH₂CH₂CF₂CH₃.

As used herein, “monocycle” includes any stable polyatomic carbon ring of up to 10 atoms and may be unsubstituted or substituted. Examples of such non-aromatic monocycle elements include but are not limited to: cyclobutyl, cyclopentyl, cyclohexyl, and cycloheptyl. Examples of such aromatic monocycle elements include but are not limited to: phenyl.

As used herein, “bicycle” includes any stable polyatomic carbon ring of up to 10 atoms that is fused to a polyatomic carbon ring of up to 10 atoms with each ring being independently unsubstituted or substituted. Examples of such non-aromatic bicycle elements include but are not limited to: decahydronaphthalene. Examples of such aromatic bicycle elements include but are not limited to: naphthalene.

As used herein, “aryl” is intended to mean any stable monocyclic, bicyclic or polycyclic carbon ring of up to 10 atoms in each ring, wherein at least one ring is aromatic, and may be unsubstituted or substituted. Examples of such aryl elements include but are not limited to: phenyl, p-toluenyl (4-methylphenyl), naphthyl, tetrahydro-naphthyl, indanyl, phenanthryl, anthryl or acenaphthyl. In cases where the aryl substituent is bicyclic and one ring is non-aromatic, it is understood that attachment is via the aromatic ring.

The term “heteroaryl”, as used herein, represents a stable monocyclic, bicyclic or polycyclic ring of up to 10 atoms in each ring, wherein at least one ring is aromatic and contains from 1 to 4 heteroatoms selected from the group consisting of O, N and S. Bicyclic aromatic heteroaryl groups include phenyl, pyridine, pyrimidine or pyridazine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition. The term “heterocycle”, “heterocyclyl” or “heterocyclic” refers to a mono- or poly-cyclic ring system which can be saturated or contains one or more degrees of unsaturation and contains one or more heteroatoms. Preferred heteroatoms include N, O, and/or S, including N-oxides, sulfur oxides, and dioxides. Preferably the ring is three to ten-membered and is either saturated or has one or more degrees of unsaturation. The heterocycle may be unsubstituted or substituted, with multiple degrees of substitution being allowed. Such rings may be optionally fused to one or more of another “heterocyclic” ring(s), heteroaryl ring(s), aryl ring(s), or cycloalkyl ring(s). Examples of heterocycles include, but are not limited to, oxetane, tetrahydrofuran, pyran, 1,4-dioxane, 1,3-dioxane, piperidine, piperazine, pyrrolidine, morpholine, thiomorpholine, tetrahydrothiopyran, tetrahydrothiophene, 1,3-oxathiolane, and the like.

As used herein, “heterocycloalkyl” is intended to mean a 3- to 10-membered nonaromatic ring containing from 1 to 4 heteroatoms selected from the group consisting of O, N and S, and includes bicyclic groups.

The term “alkylaryl” refers to alkyl groups as described above wherein one or more bonds to hydrogen contained therein are replaced by a bond to an aryl group as described above. It is understood that an “alkylaryl” group is connected to a core molecule through a bond from the alkyl group and that the aryl group acts as a substituent on the alkyl group. Examples of arylalkyl moieties include, but are not limited to, benzyl (phenylmethyl), p-trifluoromethylbenzyl (4-trifluoromethylphenylmethyl), 1-phenylethyl, 2-phenylethyl, 3-phenylpropyl, 2-phenylpropyl and the like.

As used herein, “cycloalkyl” includes cyclic rings of alkanes of three to eight total carbon atoms, or any number within this range (i.e., cyclopropyl, cyclobutyl, cyclopentyl, cyclohexyl, cycloheptyl or cyclooctyl).

The term “ester” is intended to a mean an organic compound containing the R—O—CO—R′ group.

The term “amide” is intended to a mean an organic compound containing the R—CO—NH—R′ or R—CO—N—R′R″ group.

The term “phenyl” is intended to mean an aromatic six membered ring containing six carbons.

The term “benzyl” is intended to mean a —CH₂R₁ group wherein the R₁ is a phenyl group.

The term “substitution”, “substituted” and “substituent” refers to a functional group as described above in which one or more bonds to a hydrogen atom contained therein are replaced by a bond to non-hydrogen or non-carbon atoms, provided that normal valencies are maintained and that the substitution results in a stable compound. Substituted groups also include groups in which one or more bonds to a carbon(s) or hydrogen(s) atom are replaced by one or more bonds, including double or triple bonds, to a heteroatom. Examples of substituent groups include the functional groups described above, and halogens (i.e., F, Cl, Br, and I); alkyl groups, such as methyl, ethyl, n-propyl, isopropyl, n-butyl, tert-butyl, and trifluoromethyl; hydroxyl; alkoxy groups, such as methoxy, ethoxy, n-propoxy, and isopropoxy; aryloxy groups, such as phenoxy; arylalkyloxy, such as benzyloxy (phenylmethoxy) and p-trifluoromethylbenzyloxy (4-trifluoromethylphenylmethoxy); heteroaryloxy groups; sulfonyl groups, such as trifluoromethanesulfonyl, methanesulfonyl, and p-toluenesulfonyl; nitro, nitrosyl; mercapto; sulfanyl groups, such as methylsulfanyl, ethylsulfanyl and propylsulfanyl; cyano; amino groups, such as amino, methylamino, dimethylamino, ethylamino, and diethylamino; and carboxyl. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

The compounds used in the method of the present invention may be prepared by techniques well known in organic synthesis and familiar to a practitioner ordinarily skilled in the art. However, these may not be the only means by which to synthesize or obtain the desired compounds. The compounds used in the method of the present invention may be prepared by techniques described in Vogel's Textbook of Practical Organic Chemistry, A. I. Vogel, A. R. Tatchell, B. S. Furnis, A. J. Hannaford, P. W. G. Smith, (Prentice Hall) 5^{th} Edition (1996), March's Advanced Organic Chemistry: Reactions, Mechanisms, and Structure, Michael B. Smith, Jerry March, (Wiley-Interscience) 5^{th} Edition (2007), and references therein, which are incorporated by reference herein. However, these may not be the only means by which to synthesize or obtain the desired compounds.

The various R groups attached to the aromatic rings of the compounds disclosed herein may be added to the rings by standard procedures, for example those set forth in Advanced Organic Chemistry: Part B: Reactions and Synthesis, Francis Carey and Richard Sundberg, (Springer) 5th ed. Edition. (2007), the content of which is hereby incorporated by reference.

Another aspect of the invention comprises a compound used in the method of the present invention as a pharmaceutical composition.

As used herein, the term “pharmaceutically active agent” means any substance or compound suitable for administration to a subject and furnishes biological activity or other direct effect in the treatment, cure, mitigation, diagnosis, or prevention of disease, or affects the structure or any function of the subject. Pharmaceutically active agents include, but are not limited to, substances and compounds described in the Physicians' Desk Reference (PDR Network, LLC; 64th edition; Nov. 15, 2009) and “Approved Drug Products with Therapeutic Equivalence Evaluations” (U.S. Department Of Health And Human Services, 30^{th} edition, 2010), which are hereby incorporated by reference. Pharmaceutically active agents which have pendant carboxylic acid groups may be modified in accordance with the present invention using standard esterification reactions and methods readily available and known to those having ordinary skill in the art of chemical synthesis. Where a pharmaceutically active agent does not possess a carboxylic acid group, the ordinarily skilled artisan will be able to design and incorporate a carboxylic acid group into the pharmaceutically active agent where esterification may subsequently be carried out so long as the modification does not interfere with the pharmaceutically active agent's biological activity or effect.

The compounds used in the method of the present invention may be in a salt form. As used herein, a “salt” is a salt of the instant compounds which has been modified by making acid or base salts of the compounds. In the case of compounds used to treat an infection or disease caused by a pathogen, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

As used herein, “treating” means preventing, slowing, halting, or reversing the progression of a disease or infection. Treating may also mean improving one or more symptoms of a disease or infection.

The compounds used in the method of the present invention may be administered in various forms, including those detailed herein. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutically acceptable carrier.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection, topical application, or other methods, into or onto a site of infection, all using dosage forms well known to those of ordinary skill in the pharmaceutical arts.

The compounds used in the method of the present invention can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds used in the method of the present invention may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines. The compounds may be administered as components of tissue-targeted emulsions.

The compounds used in the method of the present invention may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The compounds used in the method of the present invention may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials to make them compatible with the type of injection or delivery system chosen.

Each embodiment disclosed herein is contemplated as being applicable to each of the other disclosed embodiments. Thus, all combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

The compounds described herein can be prepared using standard techniques of synthetic organic chemistry, well known to those of ordinary skill in the art. Derivative compounds of Ariadne were prepared via the Henry reaction, lithium aluminum hydride or alane reduction, palladium cross coupling methods such as Suzuki coupling, and copper catalyzed trifluromethylation.

### Overview

Ariadne is a non-hallucinogenic analog in the mescaline lineage of psychedelics. Ariadne is a selective 5-HT2 receptor agonist, preferring the 5-HT_{2A/C} over 5-HT_{2B} receptor subtypes, with modest selectivity over 5-HT_{1E} and 5-HT_{1F} receptors (0.5-1 log), no/low activity at 5-HT_{5,6,7}, dopamine 1 and 2, and adrenergic receptors, and no relevant affinity at monoamine transporters. Ariadne, in comparison to DOM, shows lower signaling potency and efficacy in multiple signaling pathways examined (G_{q}, G₁₁, and β-arrestin2) coupled to 5-HT_{2A} receptors. We confirmed the shift in signaling for two other pairs of alpha-ethyl and alpha-methyl analogs, and an alpha-propyl analog, and provide a molecular docking rationale for the progressive decrease in signaling potency with the growing length of the alpha substituent. There is no apparent change in the relative bias of signaling between G_{q/11} and β-arrestin2, instead there is a small but consistent drop in efficacy in all signaling channels. Ariadne acts as a partial 5-HT_{2A} agonist in vivo in mice, namely, in comparison to its hallucinogenic analogs, the head twitch response (HTR) shows markedly attenuated HTR efficacy, consistent with previous studies in rabbits, cats, and dogs. Hence, we propose the lower 5-HT_{2A} receptor signaling efficacy of this compound class as an explanatory model for the lack of hallucinogenic effects of Ariadne in humans, and the dramatically attenuated hallucinosis-like effects in animals (5-HT_{2A} signaling efficacy hypothesis). We used a novel mouse model of Parkinson's disease (auxilin knock-out model) where Ariadne effected a complete rescue of severe motor deficits in this mouse line, on par to effects of L-DOPA, a notable finding considering the lack of activity of Ariadne at dopaminergic receptors and transporters. In the new light of molecular signaling mechanism, Ariadne emerges as a prototype of a new drug class, non-hallucinogenic 5HT2A/C agonists, with considerable therapeutic potential across psychiatric and neurological indications.

### EXPERIMENTAL DETAILS

General Considerations. Reagents and solvents were obtained from commercial sources and were used without further purification unless otherwise stated. The reaction solvents were purchased anhydrous and were stored under argon. Unless noted otherwise, the reactions were carried out in oven-dried glassware under an atmosphere of argon. Reactions were monitored by thin layer chromatography (TLC) using solvent mixtures appropriate to each reaction. Column chromatography was performed on silica gel (pore size 60 Å, 230-400 mesh particle size, 40-63 μm particle size). For basic amines, Et₃N was used in the mobile phase to provide better resolution when using silica gel chromatography. For preparative TLC, glass plates coated with a 1 mm silica layer were used (PLC Silica gel 60 F₂₅₄, 1 mm). Nuclear magnetic resonance spectra were recorded on Bruker 400 or 500 MHz instruments, as indicated. Chemical shifts are reported as 5 values in parts per million (ppm) referenced to CDCl₃ (¹H NMR=7.26 and ¹³C NMR=77.16) or methanol-d₄ (¹H NMR=3.31 and ¹³C NMR=49.00). Multiplicity is indicated as follows: s (singlet); d (doublet); t (triplet); dd (doublet of doublets); td (triplet of doublets); dt (doublet of triplets); dq (doublet of quartets); ddd (doublet of doublet of doublets); ddt (doublet of doublet of triplets); m (multiplet); br (broad). Low-resolution mass spectra were recorded on an Advion Advion ExpressIon CMS-L with automated TLC plate reader instrument (ionization mode: APCI+ or ESI+) or by GC-MS (ionization mode: EI). For many aldehyde and nitrostyrene intermediates that poorly ionized, materials were taken forward and characterized as amine products (either as salts or freebases). High-resolution mass spectra (HRMS) were obtained on a Waters Xevo G2 XS Q-ToF mass spectrometer in the positive electrospray ionization mode.

### Example 1 Synthesis of Ariadne and its Analogs

The majority of Ariadne analogs were prepared in two steps from commercially available aldehydes or aldehydes synthesized via established methods. The Henry reaction is a well-established means of generating nitroalkenes. We found that sonication of the appropriate substituted benzaldehyde and nitroalkane partners in the presence of butylamine catalyst in acetic acid produced the corresponding nitrostyrenes that were readily purified by either trituration with cold methanol or chromatography over a small silica pad using toluene-based eluent (Karlsen et al., 2014). The reduction of these nitrostyrenes was performed using lithium aluminum hydride in most cases. In cases where the 4-substituent was trifluoromethyl, iodo, bromo or chloro; alane (aluminum hydride) formed in situ was instead used to prevent dehalogenation.

For the 4-trifluoromethyl analog, a silver-catalyzed C—H trifluoromethylation approach employing silver fluoride and trifluoromethyltrimethylsilane was first attempted with yields consistently near 15%. While this is a serviceable method for discovery chemistry efforts, a copper (I) iodide-catalyzed approach from the corresponding 4-substituted iodo aldehyde afforded the product in 87% yield (Seo et al, 2013).

Suzuki coupling of the 4-bromo-2,5-dimethoxybenzaldehyde with potassium cyclopropyltrifluoroborate salts using Molander's conditions successfully yielded 4-cyclopropyl aldehyde (Scheme 2) (Molander et al., 2007). The synthesis of 4-methoxymethyl aldehyde was first attempted in a similar fashion but was not successful. Instead, 4-bromo-2,5-dimethoxy benzaldehyde was reduced with NaBH₄ to the alcohol, which was methylated to give the methoxymethyl aryl bromide. This was successfully formylated using nBuLi and DMF in diethyl ether (Scheme 3) (Layton et al., 2002). The Ariadne enantiomers were obtained by separation via chiral HPLC.

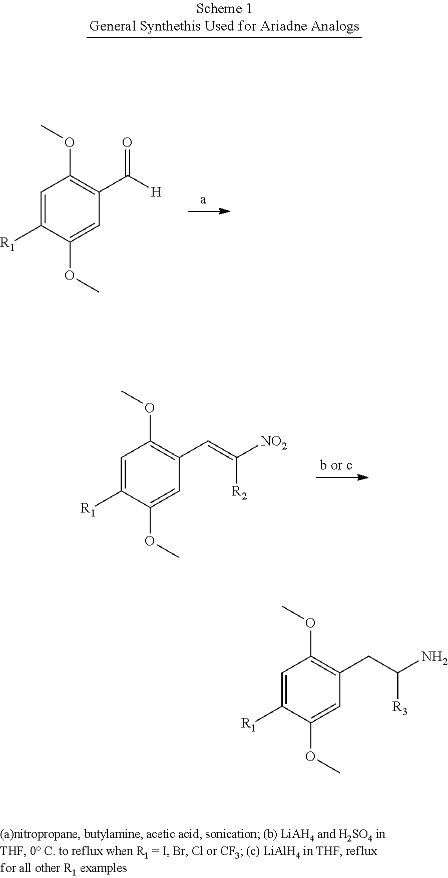

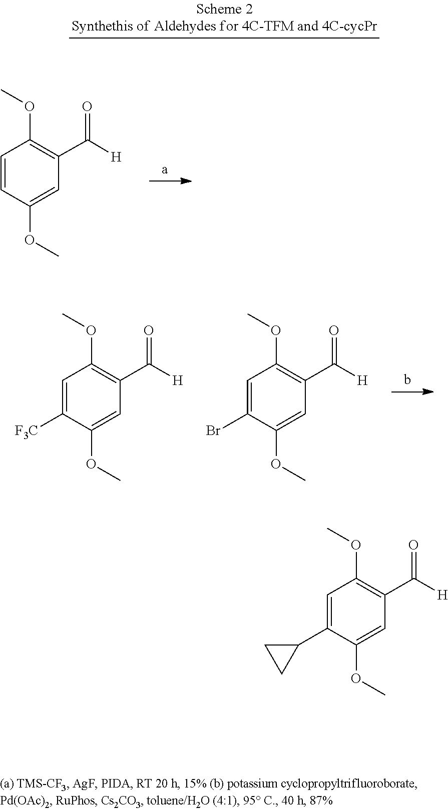 [SMILES: COc1ccc(OC)c(C=O)c1 ; COc1cc(C(F)(F)F)c(OC)cc1C=O ; COc1cc(C=O)c(OC)cc1Br ; COc1cc(C2CC2)c(OC)cc1C=O]

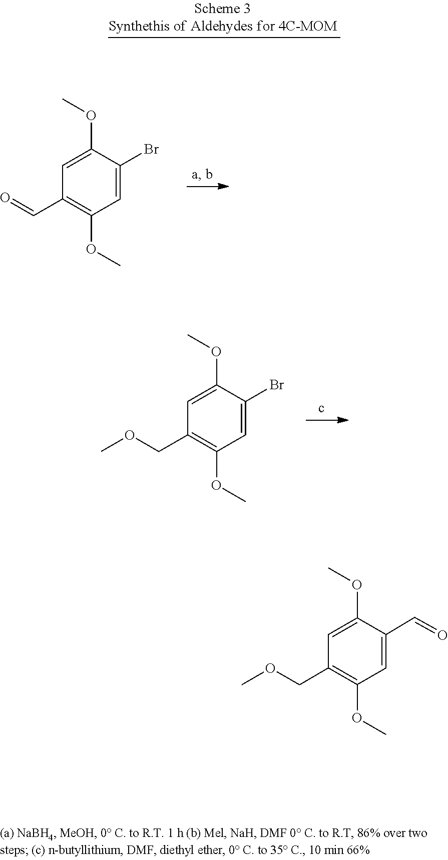

### Example 2 General Procedure A: Preparation of Nitrostyrenes

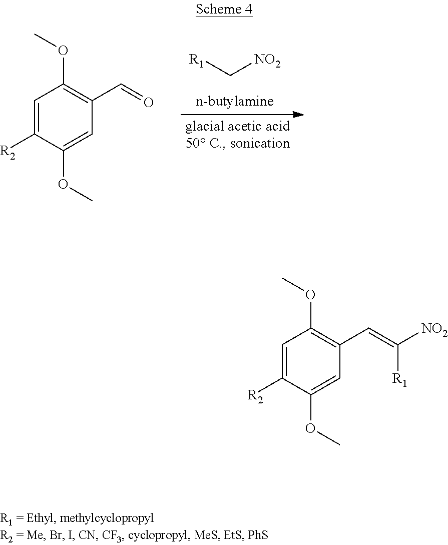

From: Shengkun, L; Kexuan, H.; Xumu, Z.; Enantioselective hydrogenation of α,β-disubstituted nitroalkenes. Chem. Commun., 2014, 50, 8878-8881. Modification: used without additional heating, vessel temperature reaches ˜50° C. over the course of reaction.

Benzaldehyde (1 eq) was added to a reaction tube with a magnetic stir bar followed by glacial acetic acid (5.5 eq) and nitropropane (1.5 eq). N-butylamine (2 eq) was then added dropwise with stirring. Reaction mixture was sealed with septum and placed in a sonicating water bath for 16 hours, then diluted with 3× the volume of toluene as the reaction mixture. This solution was transferred immediately to a short silica gel column and purified using 100% toluene as eluent.

### Example 3 General Procedure B: Preparation of Butanamine Products Using Lithium Aluminum Hydride

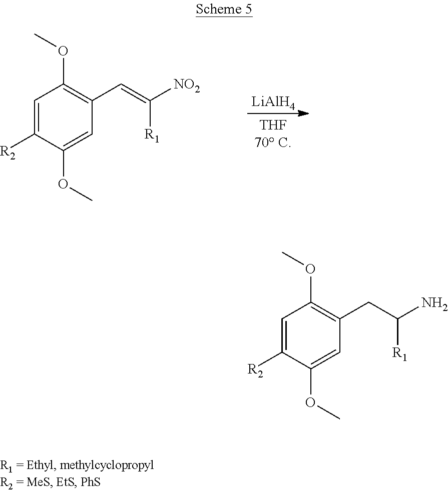

Per 1 mmol of nitrostyrene: A solution of 1 mmol of appropriate nitrostyrene in 2 mL of THF was added dropwise to a suspension of 7 mmol of LiAlH₄ in 3.5 mL of THF while stirring under an argon atmosphere. The reaction mixture was heated 18 hour at 70° C., cooled to 0° C. Reaction was carefully quenched with isopropanol (200 μL), water (200 μL), aqueous NaOH solution (200 μL, 15% NaOH), and finally more water (600 μL) then stirred vigorously for 30 min at room temperature. The suspension was vacuum filtered over Celite pad and filter cake washed 3× with ethyl acetate. Solvent was removed under reduced pressure, and the crude product was purified over with flash chromatography (9:1 ethyl acetate/methanol+2% triethylamine).

### Example 4 General Procedure C: Preparation of Butanamine Products Using Alane (Aluminum Hydride)

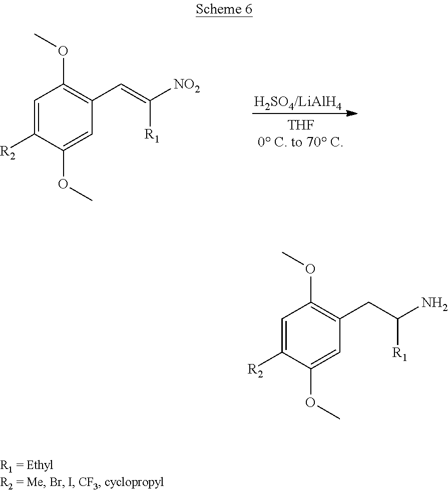

Per 1 mmol of nitrostyrene: Under argon atmosphere, 7 ml of anhydrous THF was added to oven dried scintillation vial containing a stir bar followed by lithium aluminum hydride (5 mmol) rapidly added in 3 portions. The reaction mixture was then cooled with ice water bath 5 min before adding sulfuric acid (2.5 mmol, approx. normality of 36) diluted in 2 mL dry THF in slow, dropwise fashion. This mixture was allowed to stir for 20 min in ice bath. Appropriate nitrosyrene was dissolved in 3 ml of dry THF and added dropwise (followed by 2×1 ml washes dry THF). The reaction mixture was then heated to 65° C. for 2 hr and then cooled to 0° C. with ice bath. Reaction was diluted with THF (3 mL), isopropanol was added dropwise (200 μL), water was added dropwise (200 μL), aqueous NaOH solution added dropwise (200 μL, 15% NaOH), and more water (600 μL) added dropwise and stirred vigorously 30 min at room temperature. The suspension was vacuum filtered over Celite pad and filter cake washed 3× with ethyl acetate. Solvent was removed under reduced pressure, and the crude product was purified over with flash chromatography (9:1 ethyl acetate/methanol+2% triethylamine).

### Example 5 General Procedure D: Preparation of 4-alkylthiobenzaldehydes

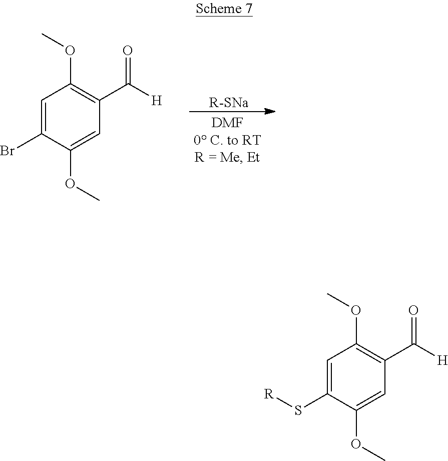

From: Synthetic Commun. 1986, 16, 565-70. Modification: Commercially available sodium alkylthiolates were used directly instead of generation in situ.

11 mmol of appropriate sodium n-alkanethiolate was added to 25 mL of anhydrous DMF under argon in one portion. This suspension was cooled in ice-bath for 10 min, then 11 mmol of 4-bromo-2,5-dimethoxybenzaldehyde was added in one portion. The reaction mixture was allowed to reach room temperature naturally and to stir for 16 hours, then it was poured into 400 ml water, off-white precipitate was filtered and allowed to air-dry.

### 2,5-dimethoxy-4-(trifluoromethyl)benzaldehyde 1 (Procedure A)

From: Chem. Commun., 2013, 49, 6385.

An oven dried 100 mL flask under argon was charged with 2,5-dimethoxybenzaldehyde (15 mmol), (diacetoxyiodo)benzene (30 mmol), trimethyl(trifluoromethyl)silane (30 mmol) and 50 mL anhydrous DMSO. This mixture was stirred at room temperature for 5 min, then AgF (3.75 mmol) was slowly added portion-wise to the stirring mixture and was stirred under argon at room temperature for 20 hour. The reaction was quenched by pouring contents of flask into 300 ml water and extracted 3×100 ml toluene. Pooled organic extracts were washed with brine and dried over MgSO₄. Following removal of solvent under reduced pressure, crude material was purified with flash chromatography using silica gel and 1:3 hexanes/DCM yielding 536 mg of product (15% yield).

### Alternate Preparation of 2,5-dimethoxy-4-(trifluoromethyl)benzaldehyde (1) (Procedure B)

From: Patent WO2022038170A1, “Therapeutic phenethylamine compositions and methods of use”.

Note: Separate reactions run in parallel 1 g×10=10 g, combined for workup and purification.

In a glass vial with screw teflon cap, methyl 2,2-difluoro-2-(fluorosulfonyl)acetate (1.31 ml, 10.27 mmol) and copper (I) iodide (98 mg, 0.514 mmol) were added to a solution of compound 1 (1 g, 3.42 mmol) in dry DMF (12 ml) at room temperature. The reaction mixture was heated at 75° C. for 16 h. The reaction mixture cooled to room temperature, filtered thorough Celite, washed with ethyl acetate (300 mL) and the solvents were evaporated in vacuo. The residue was purified by column chromatography (hexane/ethyl acetate; 9:1). The product was obtained as a white solid (6.94 g, 87%).

1H NMR (500 MHz, CDCl₃) δ 10.47 (s, 1H), 7.43 (s, 1H), 7.22 (s, 1H), 3.94 (s, 3H), 3.90 (s, 3H). 13C NMR (126 MHz, CDCl3) δ 188.92, 155.52, 151.60 (d, J=1.8 Hz), 127.59, 124.97 (q, J=31.1 Hz), 123.00 (q, 1JC-F=273.3 Hz), 111.70 (q, J=5.4 Hz), 111.16, 56.74, 56.58. 19F NMR (471 MHz, CDCl₃) δ −62.07. HRMS (ESI+): calcd. for C₁₀H₁₀F₃O₃ [M+H]+=235.0582, found [M+H]+=235.0612.

### tert-butyl (1-(4-bromo-2,5-dimethoxyphenyl)butan-2-yl) carbamate (2)

Di-tert-butyl dicarbonate (630 mg, 2.9 mmol, 1.6 eq) was added to a 20 ml scintillation vial followed by 1-(4-bromo-2,5-dimethoxyphenyl)butan-2-amine 21 (520 mg, 1.8 mmol, 1 eq), 10 mL DCM and while stirring, 400 μL (2.9 mmol, 1.6 eq) triethylamine was added dropwise and stirred at room temperature 16 hr. The reaction mixture was diluted with 10 mL DCM, then poured into saturated NH₄Cl (70 mL), washed with NaHCO₃ (70 mL), and brine (70 mL). DCM solution was dried over Na₂SO₄, dried and purified with flash chromatography using silica gel and 1:2 ethyl acetate/hexanes, yielding 400 mg of product (89% yield).

1H NMR (400 MHz, CDCl₃) δ 7.02 (s, 1H), 6.74 (s, 1H), 4.47 (s, 1H), 3.84 (s, 3H), 3.78 (s, 3H), 3.75 (bfs, 1H), 2.74 (m, 2H), 1.60-1.55 (m, 1H), 1.36 (s, 9H), 0.94 (t, J=7.4 Hz, 3H). LRMS (APCI+) calcd. For C₁₇H₂₇BrNO₄ [M+H]+=388.11, found 388.3

### tert-butyl (1-(4-cyano-2,5-dimethoxyphenyl)butan-2-yl) carbamate (3)

From: J. Med. Chem. 2010, 53, 5656-5666

Polymethylhydrosiloxane (8 mg) was added to a 20 ml oven-dried scintillation vial, followed by tert-butyl (1-(4-bromo-2,5-dimethoxyphenyl)butan-2-yl)carbamate 2 (85 mg, 0.22 mmol, 1 eq), followed by zinc cyanide (20 mg, 0.17 mmol, 0.8 eq) and tetrakis(triphenylphosphine) palladium (25 mg, 0.22 mmol, 0.1 eq), then sealed with septum and vacuum/argon cycled 3 times. 5 ml anhydrous DMF was then added and a stream of argon was passed through the suspension with a needle while stirring vigorously for 15 min. The reaction was then heated to 75° C. for 16 hours. The reaction mixture was cooled, filtered over a pad of Celite, which was rinsed with ethyl acetate (60 mL). The filtrate was washed with 100 ml saturated NaHCO₃, followed by 100 ml deionized water and finally with 100 mL brine. Organic fraction was dried over Na₂SO₄ and the concentrated crude material was subjected to flash chromatography using silica gel and 1:2 ethyl acetate/hexanes, yielding 53 mg of product (72% yield).

1H NMR (500 MHz, Chloroform-d) δ 6.95 (s, 1H), 6.82 (s, 1H), 4.42 (d, 1H), 3.88 (s, 3H), 3.80 (s, 3H), 3.78 (m, 1H), 2.82 (dd, 1H), 2.72 (dd, 1H), 1.55 (m, 1H), 1.43 (m, 1H), 1.35 (s, 9H), 0.96 (t, 3H). LRMS (APCI+) calcd. For C₁₈H₂₇N₂O₄ [M+H]⁺=335.2, found 335.2

### 4-(2-aminobutyl)-2,5-dimethoxybenzonitrile trifluoroacetate (4)

Tert-butyl (1-(4-cyano-2,5-dimethoxyphenyl)butan-2-yl)carbamate (48 mg, 143 umol, 1 eq) was dissolved in 2 ml dry DCM and 66 μL (861 umol, 6 eq) trifluoroacetic acid was added to the stirred solution. The solution was stirred overnight at room temperature before liquids were completely removed by rotary evaporation. This crude material was redissolved in 1 mL water and methanol mixture (3:7) and eluted through C18 cartridge (Thermo-Scientific 50 mg Hypersep column). Solvent was removed under reduced pressure to give 42 mg of a solid white trifluoroacetate salt product (72% yield).

1H NMR (500 MHz, DMSO-d₆) δ 7.86 (s, 3zzH), 7.36 (s, 1H), 7.13 (s, 1H), 3.87 (s, 3H), 3.79 (s, 3H), 3.36-3.29 (m, 1H), 2.92 (dd, J=13.6, 6.6 Hz, 1H), 2.83 (dd, J=13.6, 7.2 Hz, 1H), 1.57-1.48 (m, 2H), 0.93 (t, J=7.5 Hz, 3H). 13C NMR (126 MHz, DMSO-d₆) δ 158 (m), 155.63, 151.68, 132.91, 116.91, 115.85, 115.42, 99.09, 56.93, 56.75, 52.16, 33.60, 25.63, 9.81. LRMS (APCI+) calcd. For C₁₃H₁₈N₂O₂ [M+H]⁺=235.1, found 235.2

### 4-cyclopropyl-2,5-dimethoxybenzaldehyde (5)

4-Bromo-2,5-dimethoxybenzaldehyde (1.2 grams, 4.9 mmol, 1 eq), potassium cyclopropyl trifluoroborate salt (797 mg, 5.4 mmol, 1.1 eq), RuPhos (91 mg, 0.195 mmol, 0.04 eq), palladium acetate (22 mg, 0.098 mmol, 0.02 eq), and cesium carbonate (479 mg, 14.7 mmol, 3 eq) was added to oven dried vial equipped with stir-bar and sealed with septum. The mixture was vacuum/argon cycled 3 times and toluene (11 ml) and water (4 ml) were added. Reaction mixture was stirred vigorously at 95° C. for 40 hr. The reaction mixture was then cooled, filtered over Celite and rinsed with ethyl acetate. Filtrate was washed with saturated NH₄Cl solution followed by brine and organic fraction was dried over Na₂SO₄ and concentrated under reduced pressure. Crude material was subjected to flash chromatography using silica gel and hexane/DCM gradient to yield 879 mg of off-white solid (87% yield).

1H NMR (500 MHz, CDCl₃) δ 10.37 (s, 1H), 7.26 (s, 1H), 6.40 (s, 1H), 3.85 (d, J=2.9 Hz, 6H), 2.28 (tt, J=8.5, 5.3 Hz, 1H), 1.09-1.02 (m, 2H), 0.78-0.71 (m, 2H). 13C NMR (101 MHz, Chloroform-d) δ 189.15, 157.28, 152.55, 142.48, 122.43, 108.39, 108.23, 56.27, 56.21, 10.50, 9.46 (2C).

### 4-iodo-2,5-dimethoxybenzaldehyde (6)

From: Org. Biomol. Chem.; 2014, 12, 6105-6113

A mixture of 2,5-dimethoxybenzaldehyde (5.1 g, 31 mmol, 1 eq), silver nitrate (5.21 g, 31 mmol, 1 eq), and iodine (8.1 g, 31.9 mmol, 1.04 eq) in 125 mL of methanol was stirred under argon overnight. The yellow precipitate was filtered and washed with cool methanol. The remaining iodine was reduced with saturated sodium bisulfite solution by addition until the disappearance of yellow color. The solvent was removed on a rotary evaporator and the residue recrystallized from 95% ethanol to yield 8.3 grams of fluffy beige solid (93% yield).

1H NMR (500 MHz, CDCl₃) δ 10.40 (s, 1H), 7.47 (s, 1H), 7.22 (s, 1H), 3.90 (s, 3H), 3.87 (s, 3H). HRMS (ESI+): calcd. for C₉H₁₀IO₃ [M+H]+=292.9675, found [M+H]+=292.9693.

### 2,5-dimethoxy-4-propylbenzaldehyde (7)

To an oven-dried flask was added 4-bromo-2,5-dimethoxy-benzaldehyde (7.5 g, 30.6 mmol, 1 eq), XPhos (4.38, 9.18 mmol, 0.30 eq), Pd₂(dba)₃ (2.8 g, 3.06 mmol, 0.10 eq), followed by toluene (300 mL), propyl-boronic acid (4.04 g, 45.9 mmol, 1.5 eq) and K₃PO₄ (19.49 g, 91.81 mmol, 3 eq). The solution was stirred under argon at 110° C. for 18 hr. After cooling, the reaction mixture was filtered over Celite and rinsed with ethyl acetate and concentrated under reduced pressure. The crude material was subjected to flash chromatography using silica gel and ether/hexane gradient to yield 2.55 grams of pale yellow oil (40% yield).

¹H NMR (300 MHz, CDCl₃) δ 10.47-10.30 (m, 1H), 7.26 (s, 1H), 6.78 (s, 1H), 3.87 (s, 3H), 3.80 (s, 3H), 2.72-2.53 (m, 2H), 1.74-1.49 (m, 2H), 1.05-0.87 (m, 3H). ¹³C NMR (300 MHz, CDCl₃) δ 189.17, 156.69, 151.77, 140.92, 122.89, 113.91, 108.08, 56.16, 55.76, 33.07, 22.72, 13.99.

### 5-ethoxy-2-methoxybenzaldehyde (8)

5-hydroxy-2-methoxybenzaldehyde (1.5 g, 9.86 mmol, 1 eq) and anhydrous K₂CO₃ (2.73 g, 19.72 mmol, 2 eq), were added to acetonitrile (15 mL) in a 50-mL flask. The reaction mixture was heated gently at 65° C. for 30 min, iodoethane (6.34 mL, 78.87 mmol, 8 eq) was added dropwise, and was heated at 65° C. for 16 hr. After completion of the reaction, the reaction mixture concentrated partitioned between toluene and water, organics washed with brine. The crude material was subjected to flash chromatography using silica gel and hexane/DCM gradient to yield 1.27 grams of amber oil (71% yield). Taken forward without further characterization.

### 5-ethoxy-2-methoxy-4-(trifluoromethyl)benzaldehyde (9)

Prepared in the same fashion as compound 1 using 5-ethoxy-2-methoxybenzaldehyde 8 as starting material with a yield of 12%

¹H NMR (300 MHz, CDCl₃) δ 10.47 (s, 1H), 7.43 (s, 1H), 7.22 (s, 1H), 4.13 (q, J=7.0 Hz, 2H), 3.94 (s, 3H), 1.42 (t, J=9.7, 4.2 Hz, 3H).

### (4-bromo-2,5-dimethoxyphenyl)methanol (10)

To an oven-dried flask was added 4-bromo-2,5-dimethoxybenzaldehyde (3.2 g 13 mmol, 1 eq), 40 ml MeOH, 10 mL diethyl ether, 10 ml THF. After cooling with ice water bath, sodium borohydride (553 mg, 14.6 mmol, 1.12 eq) was added in 2 portions spaced 5 min. Reaction allowed to reach rt naturally and stirred 16 hr. Reaction was poured into 10% HCl and extracted with DCM. Dried over magnesium sulfate, concentrated and used without further purification with yield of 99%.

### 1-bromo-2,5-dimethoxy-4-(methoxymethyl)benzene (11)

A suspension of (4-bromo-2,5-dimethoxyphenyl)methanol (3.22 g; 13 mmol) in anhydrous dimethylformamide (90 mL) was cooled to 0° C. Then, NaH (60% dispersion in mineral oil; 1.2 mg; 30 mmol; 2.3 eq) was added. The reaction mixture was stirred for 1 hour at room temperature. Then methyl iodide (4.46 ml, 71.7 mmol; 5.5 eq) was added and the stirring was continued for further 3 hours. The mixture was added to water (250 mL) and extracted with dichloromethane (3×50 mL). The organic layer was dried over anhydrous Na₂SO₄, filtered, and evaporated in vacuo. The crude material was eluted through a silica gel pad with DCM to give a crude solid that was used without further purification with yield of 62%.

### 2,5-dimethoxy-4-(methoxymethyl)benzaldehyde (12)

To a solution of 1-bromo-2,5-dimethoxy-4-(methoxymethyl)benzene (2.07 g, 7.93 mmol, 1 eq) in anhydrous diethyl ether (125 mL) at 0° C. was added n-butyllithium (2M cyclohexanes, 4.44 ml, 8.88 mmol, 1.12 eq) dropwise over five min. The solution was stirred at 0° C. for 10 min, and DMF (1.84 ml, 23.78 mmol, 3 eq) was added. The reaction was stirred at 0° C. for 5 minutes, room temperature for 10 minutes, and heated to 40° C. for 10 minutes. The reaction was poured into 10% HCl, was extracted with DCM, and was dried over Mg₂SO₄, filtered, and concentrated to a yellow solid. Purification by silica gel chromatography provided product in 49% yield.

¹H NMR (400 MHz, CDCl₃) δ 10.43 (s, 1H), 7.28 (s, 1H), 7.12 (s, 1H), 4.52 (d, J=0.8 Hz, 2H), 3.92 (s, 3 Hz), 3.83 (s, 3H), 3.49 (s, 3H)

### Example 6 Preparation of Nitrostyrenes

### (E)-1,4-dimethoxy-2-methyl-5-(2-nitrobut-1-en-1-yl)benzene (13)

Prepared according to general procedure A using 2,5-dimethoxy-4-methylbenzaldehyde and nitropropane with yield of 80%.

¹H NMR (400 MHz, CDCl₃) δ 8.23 (s, 1H), 6.80-6.74 (m, 2H), 3.82 (d, J=8.3 Hz, 6H), 2.85 (q, J=7.4 Hz, 2H), 2.27 (s, 3H), 1.28 (t, J=7.4 Hz, 3H).

### (E)-1-bromo-2,5-dimethoxy-4-(2-nitrobut-1-en-1-yl)benzene (14)

Prepared according to general procedure A using 4-bromo-2,5-dimethoxybenzaldehyde and nitropropane with yield of 83% and taken forward without further characterization.

### (E)-1-iodo-2,5-dimethoxy-4-(2-nitrobut-1-en-1-yl)benzene (15)

Prepared according to general procedure A using 4-iodo-2,5-dimethoxybenzaldehyde and nitropropane with yield of 66%.

¹H NMR (500 MHz, CDCl₃) δ 8.10 (d, J=0.6 Hz, 1H), 7.35 (s, 1H), 6.74 (s, 1H), 3.84 (d, J=10.3 Hz, 6H), 2.80 (q, J=7.4 Hz, 2H), 1.27 (t, J=7.4 Hz, 3H).

### (E)-1-cyclopropyl-2,5-dimethoxy-4-(2-nitrobut-1-en-1-yl)benzene (16)

Prepared according to general procedure A using 4-cyclopropyl-2,5-dimethoxybenzaldehyde and nitropropane with yield of 72%.

¹H NMR (400 MHz, CDCl₃) δ 8.23 (s, 1H), 6.80 (s, 1H), 6.40 (s, 1H), 3.82 (d, J=17.0 Hz, 6H), 2.85 (q, J=7.4 Hz, 2H), 2.24 (tt, J=8.5, 5.3 Hz, 1H), 1.29 (t, J=7.4 Hz, 3H), 1.07-0.98 (m, 2H), 0.76-0.68 (m, 2H).

### (E)-1,4-dimethoxy-2-(2-nitrobut-1-en-1-yl)-5-(trifluoromethyl)benzene (17)

Prepared according to general procedure A using 2,5-dimethoxy-4-(trifluoromethyl)benzaldehyde and nitropropane with yield of 89%.

¹H NMR (500 MHz, CDCl₃) δ 8.09 (s, 1H), 7.13 (s, 1H), 6.91 (s, 1H), 3.87 (d, J=8.9 Hz, 6H), 2.78 (q, J=7.3 Hz, 2H), 1.26 (t, J=7.4 Hz, 3H).

### (E)-(2,5-dimethoxy-4-(2-nitrobut-1-en-1-yl)phenyl)(methyl)sulfane (18)

Prepared according to general procedure A using 2,5-dimethoxy-4-(methylthio)benzaldehyde and nitropropane with yield of 79%.

¹H NMR (500 MHz, Chloroform-d) δ 8.26 (s, 1H), 6.81 (s, 1H), 6.74 (s, 1H), 3.89 (d, J=1.7 Hz, 6H), 2.87 (q, J=7.4 Hz, 2H), 2.51 (s, 3H), 1.31 (t, J=7.4 Hz, 3H).

### (E)-(2,5-dimethoxy-4-(2-nitrobut-1-en-1-yl)phenyl)(ethyl)sulfane (19)

Prepared according to general procedure A using 4-(ethylthio)-2,5-dimethoxybenzaldehyde and nitropropane with yield of 69%.

¹H NMR (400 MHz, CDCl₃) δ 8.24-8.20 (m, 1H), 6.81 (d, J=6.9 Hz, 2H), 3.86 (d, J=4.2 Hz, 6H), 2.98 (q, J=7.4 Hz, 2H), 2.85 (q, J=7.4 Hz, 2H), 1.38 (t, J=7.4 Hz, 3H), 1.29 (t, J=7.4 Hz, 3H).

### (E)-1-ethyl-2,5-dimethoxy-4-(2-nitrobut-1-en-1-yl)benzene (20)

Prepared according to general procedure A using commercially available 4-ethyl-2,5-dimethoxybenzaldehyde and nitropropane with yield of 66% and taken forward without further characterization.

### (E)-1-ethoxy-4-methoxy-5-(2-nitrobut-1-en-1-yl)-2-(trifluoromethyl)benzene (21)

Prepared from 5-ethoxy-2-methoxy-4-(trifluoromethyl)benzaldehyde using general procedure A with a yield of 50% and taken forward without further characterization.

### (E)-1,4-dimethoxy-2-(methoxymethyl)-5-(2-nitrobut-1-en-1-yl)benzene (22)

Prepared from 2,5-dimethoxy-4-(methoxymethyl)benzaldehyde 12 using general procedure A with a yield of 93%.

¹H NMR (400 MHz, CDCl₃) δ 8.22 (s, 2H), 7.04 (s, 2H), 6.79 (s, 2H), 4.52 (s, 4H), 3.86 (s, 3H), 3.81 (s, 3H), 3.48 (s, 3H), 2.83 (q, J=7.4 Hz, 2H), 1.27 (t, J=7.4 Hz, 3H).

### (E)-1-(2-cyclopropyl-2-nitrovinyl)-2,5-dimethoxy-4-methylbenzene (23)

Prepared according to general procedure A from 2,5-dimethoxy-4-methylbenzaldehyde and (nitromethyl)cyclopropane with a yield of 80% and taken forward without further characterization.

### (E)-1-chloro-2,5-dimethoxy-4-(2-nitrobut-1-en-1-yl)benzene (24)

Prepared according to general procedure A using 4-chloro-2,5-dimethoxybenzaldehyde and nitropropane with yield of 92% and taken forward without further characterization.

### (E)-1,4-dimethoxy-2-(2-nitrobut-1-en-1-yl)-5-propylbenzene (25)

Prepared according to general procedure A using 2,5-dimethoxy-4-propylbenzaldehyde and nitropropane with yield of 73%.

¹H NMR (400 MHz, CDCl₃) δ 8.27 (s, 1H), 6.80 (d, J=15.4 Hz, 2H), 3.95-3.72 (m, 6H), 2.97-2.78 (m, 2H), 2.75-2.49 (m, 2H), 1.72-1.52 (m, 2H), 1.31 (t, J=7.3 Hz, 3H), 1.00 (t, J=7.4 Hz, 3H). ¹³C NMR (400 MHz, CDCl₃) δ 152.51, 152.13, 151.29, 135.64, 129.58, 118.85, 113.23, 111.36, 56.13, 55.91, 32.70, 22.99, 21.10, 14.05, 12.47.

### (E)-1,4-dimethoxy-2-(2-nitropent-1-en-1-yl)-5-propylbenzene (26)

Prepared according to general procedure A using 2,5-dimethoxy-4-propylbenzaldehyde and nitrobutane with yield of 79%.

¹H NMR (400 MHz, CDCl₃) δ 8.29 (s, 1H), 6.79 (d, J=15.6 Hz, 2H), 3.95-3.74 (m, 6H), 2.99-2.75 (m, 2H), 2.73-2.54 (m, 2H), 1.79-1.50 (m, 4H), 1.13-0.92 (m, 6H). ¹³C NMR (400 MHz, CDCl₃) δ 152.59, 151.27, 151.02, 135.62, 129.74, 118.88, 113.24, 111.24, 56.15, 55.87, 32.71, 29.57, 22.98, 21.49, 14.06 (2C).

### Example 7 Preparation of Ariadne (32) and Analogs

### 1-(4-(ethylthio)-2,5-dimethoxyphenyl)butan-2-amine (27)

Prepared from (E)-(2,5-dimethoxy-4-(2-nitrobut-1-en-1-yl)phenyl) (ethyl)sulfane using general procedure B with yield of 23%.

¹H NMR (400 MHz, CDCl₃) δ 6.84 (s, 1H), 6.69 (s, 1H), 3.84 (s, 3H), 3.78 (s, 3H), 2.91 (q, J=7.4 Hz, 3H), 2.79 (dd, J=13.1, 4.6 Hz, 1H), 2.43 (dd, J=13.1, 8.6 Hz, 1H), 1.57-1.45 (m, 1H), 1.36 (dd, J=14.2, 6.9 Hz, 2H), 1.29 (t, J=7.4 Hz, 4H), 0.97 (t, J=7.4 Hz, 3H). ¹³C NMR (101 MHz, Chloroform-d) δ 151.86, 151.81, 127.46, 122.07, 114.17, 113.88, 56.46, 56.15, 52.92, 38.77, 30.57, 26.93, 14.29, 10.63. LRMS (APCI+) calcd. For C₁₄H₂₄NO₂S [M+H]⁺=270.1, found 270.1.

### 1-(2,5-dimethoxy-4-(methylthio)phenyl)butan-2-amine (28)

Prepared from (E)-(2,5-dimethoxy-4-(2-nitrobut-1-en-1-yl)phenyl)(methyl)sulfane using general procedure B with yield of 70%.

¹H NMR (400 MHz, Methanol-d₄) δ 6.77 (s, 1H), 6.68 (s, 1H), 3.85 (s, 3H), 3.80 (s, 3H), 2.92 (m, J=8.5, 7.4, 4.9 Hz, 1H), 2.84-2.75 (m, 1H), 2.44 (s, 4H), 1.52 (m, 1H), 1.36 (m, 1H), 0.98 (t, J=7.4 Hz, 3H). LRMS (APCI+) calcd. For C₁₃H₂₂NO₂S [M+H]⁺=256.1, found 256.1.

### 1-(2,5-dimethoxy-4-(trifluoromethyl)phenyl)butan-2-amine (29)

Prepared from (E)-1,4-dimethoxy-2-(2-nitrobut-1-en-1-yl)-5-(trifluoromethyl)benzene using general procedure C with yield of 24%.

¹H NMR (500 MHz, CDCl₃) δ 7.03 (s, 1H), 6.84 (s, 1H), 3.86 (s, 3H), 3.80 (s, 3H), 3.00-2.92 (m, 1H), 2.85 (dd, J=13.0, 4.7 Hz, 1H), 2.49 (dd, J=13.0, 8.6 Hz, 1H), 1.59-1.46 (m, 1H), 1.44-1.30 (m, 1H), 0.98 (t, J=7.4 Hz, 3H). ¹³C NMR (126 MHz, CDCl₃) δ 151.42, 151.38, 133.98, 123.89 (q, ¹J_{C-F}=272.1 Hz), 117.09 (q, J=31.1 Hz), 116.08, 109.49 (q, J=5.4 Hz), 56.89, 56.22, 52.94, 39.12, 30.85, 10.73. ¹⁹F NMR (471 MHz, CDCl₃) δ −60.85. HRMS (ESI): calcd. for C₁₃H₁₉F₃NO₂ [M+H]⁺=278.1368, found [M+H]⁺=278.1374.

### 1-(4-cyclopropyl-2,5-dimethoxyphenyl)butan-2-amine (30)

Prepared from (E)-1-cyclopropyl-2,5-dimethoxy-4-(2-nitrobut-1-en-1-yl)benzene using general procedure C in yield of 44%.

¹H NMR (400 MHz, Methanol-d₄) δ 6.71 (s, 1H), 6.43 (s, 1H), 3.80 (s, 3H), 3.73 (s, 3H), 2.89 (ddt, J=8.1, 7.1, 5.3 Hz, 1H), 2.77 (dd, J=13.1, 5.2 Hz, 1H), 2.45 (dd, J=13.1, 8.1 Hz, 1H), 2.12 (tt, J=8.6, 5.4 Hz, 1H), 1.56-1.30 (m, 2H), 0.97 (t, J=7.5 Hz, 3H), 0.91-0.85 (m, 2H), 0.67-0.58 (m, 2H). HRMS (ES+) calcd. For C₁₅H₂₃NO₂ [M+H]⁺=250.1807, found 250.1817.

### 1-(4-bromo-2,5-dimethoxyphenyl)butan-2-amine (31)

Prepared from (E)-1-bromo-2,5-dimethoxy-4-(2-nitrobut-1-en-1-yl)benzene using general procedure C with yield of 90%.

¹H NMR (500 MHz, CDCl₃) δ 7.03 (s, 1H), 6.75 (s, 1H), 3.84 (s, 3H), 3.76 (s, 3H), 2.92 (s, 1H), 2.78 (dd, J=13.1, 4.6 Hz, 1H), 2.42 (dd, J=13.1, 8.6 Hz, 1H), 1.55-1.45 (m, 1H), 1.34 (dt, J=13.5, 7.3 Hz, 1H), 0.97 (t, J=7.5 Hz, 3H). ¹³C NMR (126 MHz, Chloroform-d) δ 152.22, 149.78, 128.60, 115.89, 115.41, 108.90, 56.97, 56.09, 52.69, 38.84, 10.61. LRMS (APCI+) calcd. For C₁₂H₁₉BrNO₂ [M+H]⁺=288.1, found 288.1.

### 1-(2,5-dimethoxy-4-methylphenyl)butan-2-amine (32) (Ariadne)

Prepared from (E)-1,4-dimethoxy-2-methyl-5-(2-nitrobut-1-en-1-yl)benzene using general procedure C with yield of 92%.

¹H NMR (400 MHz, CDCl₃) δ 6.67 (d, J=11.3 Hz, 2H), 3.77 (d, J=8.0 Hz, 6H), 3.00-2.90 (m, 1H), 2.81 (dd, J=13.2, 4.6 Hz, 1H), 2.46 (dd, J=13.2, 8.6 Hz, 1H), 2.26 (s, 2H), 2.21 (s, 3H), 1.61-1.46 (m, 1H), 1.39 (dt, J=13.6, 7.3 Hz, 1H), 0.98 (t, J=7.4 Hz, 3H). LRMS (APCI+) calcd. For C₁₃H₂₂NO₂ [M+H]⁺=224.2, found 224.4. Optical rotation: Enantiomers were resolved from racemic freebase using chiral supercritical fluid chromatography (see details below, example 19) and values collected on Jasco P-2000 Digital Polarimeter. (S)-Ariadne: +30.5, (R)-Ariadne: −31.5. Averaged values of three runs each, freebases dissolved in anhydrous methanol at a concentration of 50% (w/v), average temperature of 27.7° C. and path length of 100 mm.

### 1-(2,5-dimethoxy-4-(methylsulfonyl)phenyl)butan-2-amine trifluoroacetate (33)

1-(2,5-dimethoxy-4-(methylthio)phenyl)butan-2-amine (25 mg, 98 umol, 1 eq) was dissolved in 2 ml ethyl acetate. The vial was cooled in dry ice/acetonitrile bath and mCPBA was added in one portion (71 mg, 411 umol, 4.2 eq). The reaction reached room temperature and stirred for 20 hours. Reaction mixture was diluted with ethyl acetate, washed with sodium sulfite (sat., 2×5 mL), sodium bicarbonate (sat., 1×5 mL), and water (1×5 mL). The extract was dried over Na2SO4, filtered and evaporated to dryness. The resulting oily residue was purified by addition of trifluoroacetic acid and methanol and elutied through Thermo Scientific™ HyperSep™ C18 cartridge and evaporated to give product in 46% yield.

¹H NMR (400 MHz, Methanol-d₄) δ 7.47 (s, 2H), 7.16 (s, 2H), 3.97 (s, 6H), 3.89 (s, 6H), 3.52-3.41 (m, 2H), 3.23 (s, 6H), 3.07 (d, J=6.0 Hz, 2H), 2.95 (dd, J=13.7, 7.6 Hz, 2H), 1.76-1.60 (m, 4H), 1.09-1.04 ¹³C NMR (101 MHz, Methanol-d₄) δ 151.32, 132.32 (s), 127.52, 116.37, 110.53, 56.01, 55.19, 52.88, 41.82, 33.23, 25.45, 8.46. LRMS (APCI+) calcd. For C₁₃H₂₂NO₄S [M+H]⁺=288.1, found 288.5.

### 1-(4-iodo-2,5-dimethoxyphenyl)butan-2-amine (34)

Prepared from (E)-1-iodo-2,5-dimethoxy-4-(2-nitrobut-1-en-1-yl)benzene using general procedure C with a yield of 82%.

¹H NMR (400 MHz, CDCl₃) δ 6.68 (d, J=5.6 Hz, 4H), 3.78 (d, J=6.1 Hz, 11H), 3.05 (ddd, J=13.0, 7.6, 5.4 Hz, 2H), 2.84 (dd, J=13.3, 5.2 Hz, 2H), 2.57 (dd, J=13.3, 8.2 Hz, 3H), 1.61-1.43 (m, 4H), 1.00 (t, J=7.5 Hz, 6H). LRMS (APCI+) calcd. For C₁₂H₁₉INO₂ [M+H]⁺=336.0, found 336.2

### 1-(4-ethyl-2,5-dimethoxyphenyl)butan-2-amine (35)

Prepared from (E)-1-ethyl-2,5-dimethoxy-4-(2-nitrobut-1-en-1-yl)benzene using general procedure B. Freebase was dissolved in anhydrous diethyl ether and treated with 2N HCl in ether and filtered to produce hydrochloride salt in 55% total yield.

¹H NMR (300 MHz, MeOD) δ 6.77 (d, J=15.1 Hz, 2H), 3.78 (d, J=6.9 Hz, 6H), 3.37 (dd, J=13.2, 6.5 Hz, 1H), 2.88 (ddd, J=21.2, 13.8, 6.7 Hz, 2H), 2.59 (q, J=7.5 Hz, 2H), 1.74-1.60 (m, 2H), 1.14 (t, J=7.5 Hz, 3H), 1.03 (t, J=7.5 Hz, 3H) LRMS (APCI+) calcd. For C₁₄H₂₃NO₂ [M+H]⁺=238.2, found 238.4

### 1-(5-ethoxy-2-methoxy-4-(trifluoromethyl)phenyl)butan-2-amine (36)

Prepared from (E)-1-ethoxy-4-methoxy-5-(2-nitrobut-1-en-1-yl)-2-(trifluoromethyl)benzene, following general procedure C in a yield of 56%

¹³C NMR (101 MHz, CDCl₃) δ 151.12 (s), 150.59 (s), 133.44 (s), 125.04 (s), 122.33 (s), 117.24 (s), 109.16 (dd), 65.44 (s), 55.97 (s), 52.72 (s), 38.69 (s), 30.37 (s), 14.76 (s), 10.50 (s). LRMS (APCI+) calcd. For C₁₄H₂₁F₃NO₂ [M+H]⁺=292.2, found 292.4.

### 1-(2,5-dimethoxy-4-(methoxymethyl)phenyl)butan-2-amine (37)

Prepared from (E)-1,4-dimethoxy-2-(methoxymethyl)-5-(2-nitrobut-1-en-1-yl)benzene according to general procedure B with yield of 87%.

¹H NMR (400 MHz, CDCl₃) δ 6.90 (s, 1H), 6.70 (s, 1H), 4.47 (s, 2H), 3.79 (s, 6H), 3.43 (s, 3H), 3.01-2.88 (m, 1H), 2.82 (dd, 1H), 2.46 (dd, 1H), 1.67 (bs, 2H), 1.57-1.44 (m, 1H), 1.42-1.26 (m, 1H), 0.97 (t, J=7.4 Hz, 3H). ¹³C NMR (101 MHz, CDCl₃) δ 151.93, 150.95, 128.27, 125.25, 114.20, 112.03, 69.52, 58.58, 56.34, 56.13, 53.08, 38.93, 30.60, 10.74. HRMS (ESI+) calcd. For C₁₄H₂₃NO₃ [M+H]⁺ 254.1756, found 254.1764

### 1-cyclopropyl-2-(2,5-dimethoxy-4-methylphenyl)ethan-1-amine (38)

Prepared from ((E)-1-(2-cyclopropyl-2-nitrovinyl)-2,5-dimethoxy-4-methylbenzene using general procedure B with yield of 41% (as HCl salt).

¹H NMR (400 MHz, MeOD) 6.82 (s, 1H), 6.79 (s, 1H), 3.90-3.73 (m, 6H), 3.39-3.28 (m, 4H), 3.12-2.93 (m, 2H), 2.72-2.60 (m, 1H), 2.21 (s, 3H), 1.08-0.91 (m, 1H), 0.72-0.54 (m, 1H), 0.47-0.36 (m, 1H), 0.23-0.13 (m, 1H). ¹³C NMR (400 MHz, MeOD) δ 151.71, 151.31, 126.40, 121.40, 113.69, 113.46, 57.87, 55.12, 54.88, 34.25, 14.89, 13.37, 3.38, 2.75. HRMS (ES+) calcd. For C₁₄H₂₁NO₂ [M+Na]⁺ 258.147, found 258.1465

### 1-(2,5-dimethoxy-4-methylphenyl) pentan-2-amine (39)

Prepared from 2,5-dimethoxy-4-methylbenzaldehyde using general procedures A directly followed by B with yield of 2% (over 2 steps).

¹H NMR (500 MHz, MeOD) δ 6.77 (s, 1H), 6.70 (s, 1H), 3.83-3.72 (m, 6H), 3.04-2.97 (m, 1H), 2.81 (dd, J=13.1, 5.2 Hz, 1H), 2.46 (dd, J=13.1, 8.2 Hz, 1H), 1.54-1.28 (m, 4H), 1.00-0.91 (m, 3H). ¹³C NMR (500 MHz, MeOD) δ 151.48, 151.42, 125.03, 124.97, 113.50, 113.46, 61.42, 55.04, 50.78, 38.79, 37.76, 28.78, 14.84, 13.11. LRMS (APCI+) calcd. For C₁₆H₂₈NO₂ [M+H]⁺=266.2 found 266.2

### 1-(2,6-dimethoxy-4-methylphenyl)butan-2-amine (40)

Prepared from 2,6-dimethoxy-4-methylbenzaldehyde using general procedures A directly followed by B with yield of 1.3% (over 2 steps).

¹H NMR (500 MHz, MeOD) δ 6.47 (s, 2H), 3.80 (s, 6H), 2.98-2.90 (m, 1H), 2.76 (dd, J=13.0, 5.2 Hz, 1H), 2.61 (dd, J=13.0, 8.1 Hz, 1H), 2.34 (s, 3H), 1.50-1.34 (m, 4H), 0.97-0.90 (m, 3H). ¹³C NMR (500 MHz, MeOD) δ 158.36(2C), 137.52, 112.07, 104.22(2C), 54.56(2C), 50.90, 38.81, 29.78, 20.66, 18.96, 13.09. LRMS (APCI+) calcd. For C₁₃H₂₂NO₂ [M+H]⁺=224.2, found 224.5.

### 1-(4-chloro-2,5-dimethoxyphenyl)butan-2-amine (41)

Prepared from (E)-1-chloro-2,5-dimethoxy-4-(2-nitrobut-1-en-1-yl)benzene using general procedures A with yield of 51%.

¹H NMR (400 MHz, MeOD) δ 7.06 (s, 1H), 7.00 (s, 1H), 3.87 (s, 3H), 3.84 (s, 3H), 3.46-3.37 (m, 1H), 3.01 (dd, J=13.9, 6.2 Hz, 1H), 2.89 (dd, J=13.8, 7.4 Hz, 1H), 1.76-1.61 (m, 2H), 1.07 (t, J=7.5 Hz, 3H). ¹³C NMR (400 MHz, MeOD) δ 151.86, 149.26, 123.46, 121.57, 115.91, 113.05, 56.03, 55.23, 53.25, 32.89, 25.29, 8.57. LRMS (APCI+) calcd. For C₁₂H₁₉ClNO₂ [M+H]⁺=244.1, found 244.1

### 1-(2,5-dimethoxy-4-propylphenyl)butan-2-amine (42)

Prepared from corresponding styrene using general procedures A with yield of 54% (as the HCl salt). ¹H NMR (400 MHz, MeOD) δ 6.82 (s, 1H), 6.78 (s, 1H), 3.82 (s, 3H), 3.80 (s, 3H), 3.57 (h, J=6.7 Hz, 1H), 2.95 (dd, J=13.5, 6.7 Hz, 1H), 2.85 (dd, J=13.5, 7.0 Hz, 1H), 2.63-2.56 (m, 2H), 1.68-1.54 (m, 2H), 1.29 (d, J=6.6 Hz, 3H), 0.96 (t, J=7.4 Hz, 3H). ¹³C NMR (400 MHz, MeOD) δ 151.54, 151.36, 131.13, 121.57, 113.84, 112.96, 55.17, 54.92, 48.08, 35.32, 32.04, 22.98, 17.23, 12.92. LRMS (APCI+) calcd. For C₁₅H₂₆NO₂ [M+H]⁺=252.2, found 252.2.

### 1-(2,5-dimethoxy-4-propylphenyl) pentan-2-amine (43)

Prepared from corresponding styrene using general procedures A with yield of 41% (as the HCl salt). ¹H NMR (400 MHz, MeOD) δ 6.82 (s, 1H), 6.79 (s, 1H), 3.83 (s, 3H), 3.80 (s, 3H), 3.51-3.42 (m, 1H), 2.99 (dd, J=13.9, 6.0 Hz, 1H), 2.85 (dd, J=13.8, 7.4 Hz, 1H), 2.63-2.55 (m, 2H), 1.68-1.56 (m, 4H), 1.54-1.40 (m, 2H), 1.03-0.92 (m, 6H). ¹³C NMR (400 MHz, MeOD) δ 151.57, 151.41, 131.16, 121.49, 113.88, 112.99, 55.18, 54.93, 51.94, 34, 47, 33.49, 32.04, 22.98, 18.19, 12.92, 12.65. LRMS (APCI+) calcd. For C₁₆H₂₈NO₂ [M+H]⁺=266.2, found 266.2

### 1-(2,5-dimethoxy-4-propylphenyl) propan-2-amine (44) (DOPR)

Prepared from corresponding styrene using general procedures A with yield of 41% (as the HCl salt). ¹H NMR (400 MHz, MeOD) δ 6.82 (s, 1H), 6.78 (s, 1H), 3.82 (s, 3H), 3.80 (s, 3H), 3.57 (h, J=6.7 Hz, 1H), 2.95 (dd, J=13.5, 6.7 Hz, 1H), 2.85 (dd, J=13.5, 7.0 Hz, 1H), 2.63-2.56 (m, 2H), 1.68-1.54 (m, 2H), 1.29 (d, J=6.6 Hz, 3H), 0.96 (t, J=7.4 Hz, 3H). ¹³C NMR (400 MHz, MeOD) δ 151.54, 151.36, 131.13, 121.57, 113.84, 112.96, 55.17, 54.92, 48.08, 35.32, 32.04, 22.98, 17.23, 12.92. LRMS (APCI+) calcd. For C₁₄H₂₄NO₂ [M+H]⁺=238.3, found 238.4

### Scheme for Synthesis of (E)-1,4-dimethoxy-2-(2-nitrobut-1-en-1-yl)benzene (47)

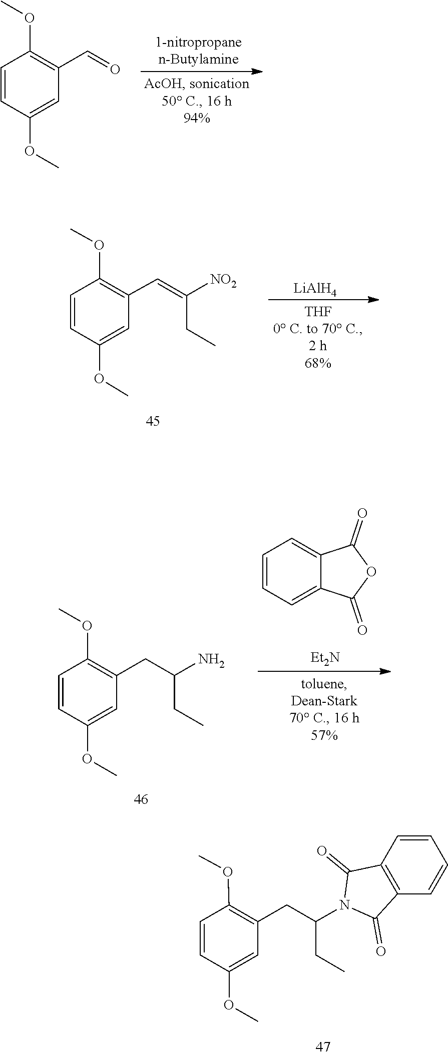 [SMILES: COc1ccc(OC)c(C=O)c1 ; CC/C(=C\c1cc(OC)ccc1OC)[N+](=O)[O-] ; "LiAlH4" ; CCC(N)Cc1cc(OC)ccc1OC ; O=C1OC(=O)c2ccccc21 ; CCC(Cc1cc(OC)ccc1OC)N1C(=O)c2ccccc2C1=O]

### (E)-1,4-dimethoxy-2-(2-nitrobut-1-en-1-yl)benzene (45)

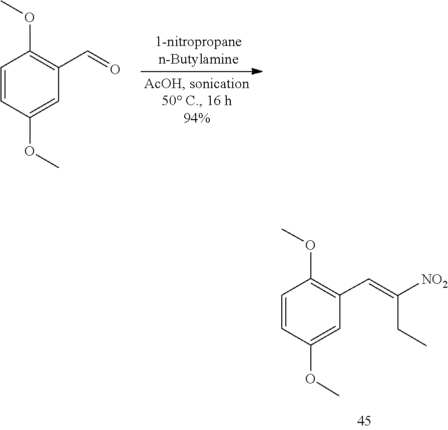 [SMILES: COc1ccc(OC)c(C=O)c1 ; CC/C(=C\c1cc(OC)ccc1OC)[N+](=O)[O-]]

n-Butylamine (11.90 mL, 120.358 mmol) was added dropwise at room temperature to a stirred solution of 2,5-dimethoxybenzaldehyde (10 g, 60.179 mmol), 1-nitropropane (11.30 mL, 120.358 mmol) in glacial acetic acid (50 mL) at room temperature. The reaction mixture was sealed with septum and placed in a sonicating water bath at 50° C. for 16 hours, then diluted with toluene and hexanes (1:1 mixture, 200 mL) and transferred immediately to a short silica gel column and purified using toluene and hexanes (1:1 mixture, 200 mL) as an eluent. The solvents were evaporated in vacuo to afford (E)-1,4-dimethoxy-2-(2-nitrobut-1-en-1-yl)benzene. The product was obtained as yellow wax (13.40 g, 94%). The obtained compound was used into the next step without further purification.

### 1-(2,5-dimethoxyphenyl)butan-2-amine (46)

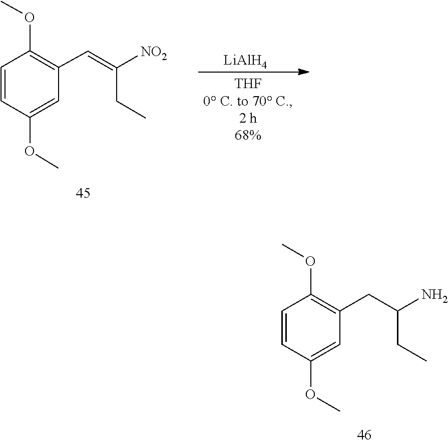 [SMILES: CC/C(=C\c1cc(OC)ccc1OC)[N+](=O)[O-] ; "LiAlH4" ; CCC(N)Cc1cc(OC)ccc1OC]

LiAlH₄ (10.72 g, 282.39 mmol) was added portion wise (3 portion) to a dry THF (300 mL) at room temperature under argon atmosphere. The reaction mixture cooled to 0° C. and a solution of (E)-1,4-dimethoxy-2-(2-nitrobut-1-en-1-yl)benzene (13.40 g, 56.478 mmol) in dry THF (75 mL) was added dropwise at 0° C. to the reaction mixture over 30 min. The reaction mixture was stirred at 70° C. for 2 h. The Reaction was cooled at 0° C. and carefully quenched with isopropanol (20 mL), water (10 mL), aqueous 15% NaOH solution (10 mL), and water (20 mL) then vigorously stirred for 14 hour at room temperature. The suspension was vacuum filtered over celite, and filter cake washed with ethyl acetate (3×200 mL) and methanol (200 mL). The solvents were evaporated in vacuo, and the residue was purified over with flash chromatography (dichloromethane/methanol; 95:5). The product was obtained as a colorless wax (8 g, 68%, contaminated with demethylated side-product). The obtained product was used into the next step without further purification.

¹H NMR (300 MHz, CDCl₃) δ (ppm) 6.81-6.69 (m, 3H), 3.77 (s, 3H), 3.76 (s, 3H), 3.03-2.90 (m, 1H), 2.81 (dd, J=13.1, 4.8 Hz, 1H), 2.48 (dd, J=13.1, 8.4 Hz, 1H), 1.83 (br s, 2H), 1.60-1.45 (m, 1H), 1.43-1.31 (m, 1H), 0.98 (t, J=7.4 Hz, 3H).

LRMS (APCI): calcd. for C₁₂H₂₀NO₂ [M+H]⁺=210.14, found [M+H]⁺=210.2.

### 2-(1-(2,5-dimethoxyphenyl)butan-2-yl)isoindoline-1,3-dione (47)

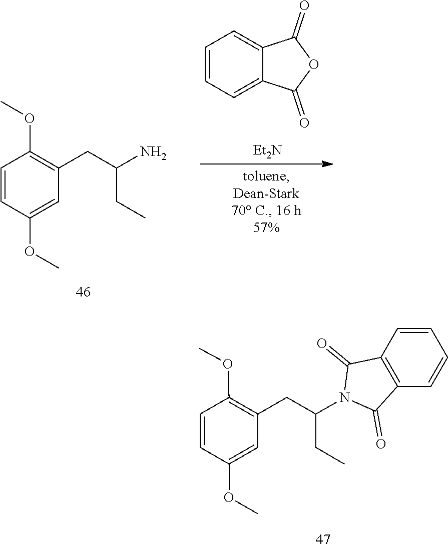 [SMILES: CCC(N)Cc1cc(OC)ccc1OC ; O=C1OC(=O)c2ccccc21 ; CCC(Cc1cc(OC)ccc1OC)N1C(=O)c2ccccc2C1=O]

Triethylamine (2.81 mL, 38.224 mmol) and phthalic anhydride (5.66 g, 38.224 mmol) were added to a solution of 1-(2,5-dimethoxyphenyl)butan-2-amine (8 g, 38.224 mmol) in dry toluene (100 mL) at room temperature under argon atmosphere. The reaction mixture was stirred at reflux temperature using Dean-Stark apparatus for 16 hours. The reaction mixture was cooled to room temperature, diluted with ethyl acetate (150 mL), and washed with aqueous 10% HCl solution (100 mL), water (100 mL), aqueous 10% NaOH solution (100 mL) and water (100 mL). The organic extracts were dried over MgSO₄, filtered, and the solvent was evaporated in vacuo. The residue obtained after the workup was purified by column chromatography (hexane/ethyl acetate, 80:20). The product was obtained as a white solid (7 g, 54%).

¹H NMR (500 MHz, CDCl₃) δ (ppm) 7.74 (dd, J=5.5, 3.0 Hz, 2H), 7.65 (dd, J=5.5, 3.1 Hz, 2H), 6.67 (d, J=8.8 Hz, 1H), 6.63-6.59 (m, 2H), 4.48 (tt, J=10.2, 5.3 Hz, 1H), 3.70 (s, 3H), 3.56 (s, 3H), 3.23 (dd, J=13.5, 9.8 Hz, 1H), 3.10 (dd, J=13.4, 5.4 Hz, 1H), 2.26-2.13 (m, 1H), 1.92-1.80 (m, 1H), 0.90 (t, J=7.4 Hz, 3H).

¹³C NMR (126 MHz, CDCl₃) δ (ppm) 168.80, 153.31, 152.13, 133.75, 132.01, 128.12, 123.01, 116.72, 112.59, 111.32, 55.89, 55.73, 53.83, 33.89, 25.46, 11.33.

HRMS (ESI): calcd. for C₂₀H₂₁NO₄Na [M+Na]⁺=362.1368, found [M+Na]⁺=362.1378.

### Scheme for Synthesis of 1-(4-(1,1-difluoroethyl)-2,5-dimethoxyphenyl)butan-2-amine (50)

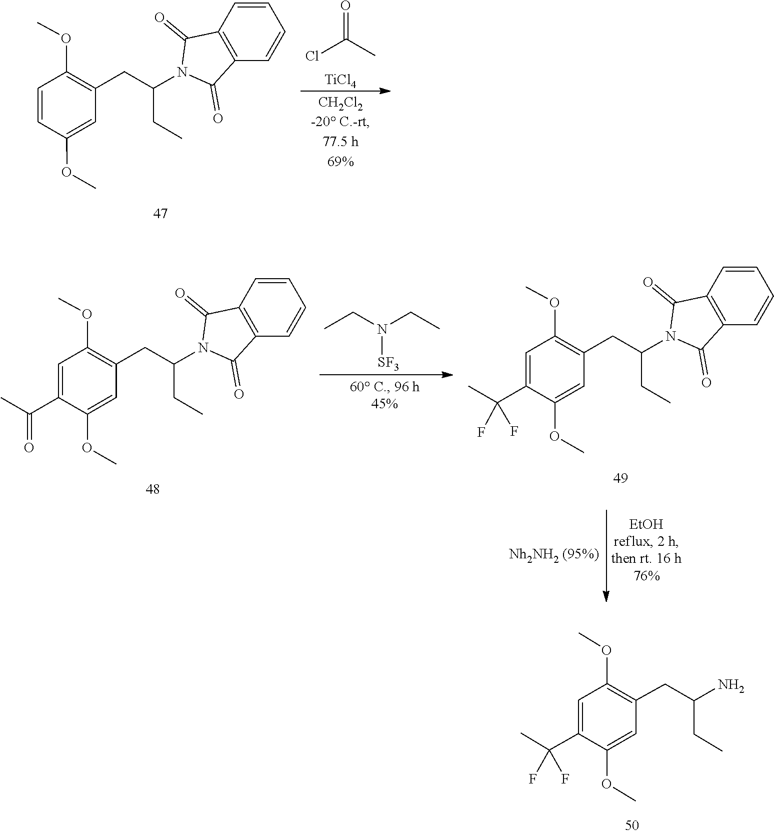

### 2-(1-(4-acetyl-2,5-dimethoxyphenyl)butan-2-yl)isoindoline-1,3-dione (48)

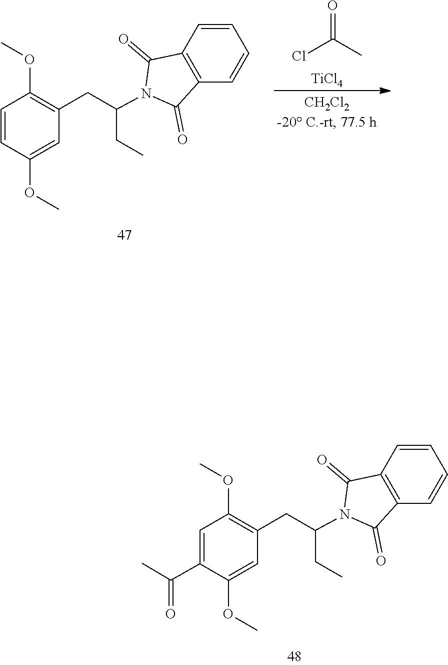

TiCl₄ (5.48 mL, 5.48 mmol; 1M in dichloromethane) was added dropwise to a cooled (−20° C.) solution of 2-(1-(2,5-dimethoxyphenyl)butan-2-yl)isoindoline-1,3-dione (1 g, 2.946 mmol) in dry dichloromethane (30 mL) under argon atmosphere. Then acetyl chloride (251 μL, 3.536 mmol) was added dropwise to the reaction mixture, and the resulting mixture was stirred at −20° C. for 30 minutes. Then the reaction mixture was warmed to room temperature and stirred for 48 hours. The reaction mixture was quenched with water (40 mL) and extracted with dichloromethane (2×80 mL). The combined organic extracts were dried over MgSO₄, filtered, and the solvent was evaporated in vacuo. The residue obtained after the workup was purified by column chromatography (hexane/ethyl acetate, 80:20). The product was obtained as a white solid (770 mg, 69%).

¹H NMR (500 MHz, CDCl₃) δ (ppm) 7.78-7.71 (m, 2H), 7.70-7.63 (m, 2H), 7.19 (s, 1H), 6.68 (s, 1H), 4.52 (tt, J=10.3, 5.2 Hz, 1H), 3.73 (s, 3H), 3.65 (s, 3H), 3.34 (dd, J=13.5, 10.3 Hz, 1H), 3.11 (dd, J=13.5, 5.1 Hz, 1H), 2.51 (s, 3H), 2.26-2.15 (m, 1H), 1.94-1.82 (m, 1H), 0.92 (t, J=7.4 Hz, 3H). ¹³C NMR (126 MHz, CDCl₃) δ (ppm) 198.85, 168.73, 153.49, 151.70, 133.93, 133.74, 131.89, 126.32, 123.09, 114.94, 111.59, 56.05, 55.90, 53.37, 33.82, 32.04, 25.72, 11.31.

HRMS (ESI): calcd. for C₂₂H₂₃NO₅Na [M+Na]⁺=404.1474, found [M+Na]⁺=404.1494.

### 2-(1-(4-(1,1-difluoroethyl)-2,5-dimethoxyphenyl)butan-2-yl)isoindoline-1,3-dione (49)

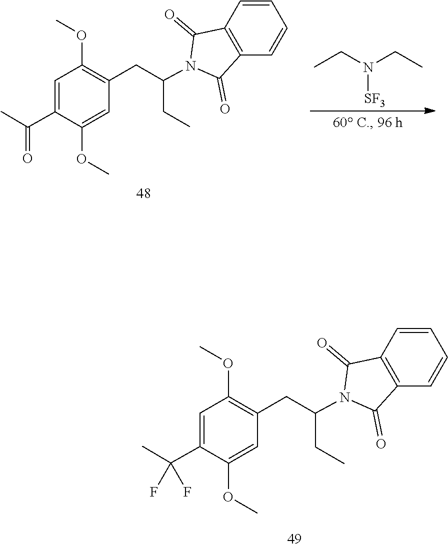

In a glass vial, diethylaminosulfur trifluoride (2.50 mL, 18.876 mmol) was added to 2-(1-(4-acetyl-2,5-dimethoxyphenyl)butan-2-yl)isoindoline-1,3-dione (720 mg, 1.888 mmol) under argon atmosphere. The resulting reaction mixture was stirred at 60° C. for 96 hours. The rection mixture was carefully quenched with aqueous saturated NaHCO₃ solutions (70 mL) and extracted with ethyl acetate (3×80 mL). The combined organic extracts were dried over MgSO₄, filtered, and the solvent was evaporated in vacuo. The residue obtained after the workup was purified by column chromatography (hexane/ethyl acetate, 90:10), and then preparative TLC (hexane/ethyl acetate, 90:10, repeated elution 3-times). The product was obtained as a white solid (340 mg, 45%).

¹H NMR (500 MHz, CDCl₃) δ (ppm) 7.79-7.71 (m, 2H), 7.70-7.62 (m, 2H), 6.92 (s, 1H), 6.66 (s, 1H), 4.51 (tt, J=10.3, 5.3 Hz, 1H), 3.75 (s, 3H), 3.59 (s, 3H), 3.29 (dd, J=13.6, 10.1 Hz, 1H), 3.13 (dd, J=13.6, 5.3 Hz, 1H), 2.25-2.14 (m, 1H), 1.91-1.82 (m, 1H), 1.89 (t, J=18.8 Hz, 3H), 0.91 (t, J=7.4 Hz, 3H).

¹³C NMR (126 MHz, CDCl₃) δ (ppm) 168.80, 151.56, 150.32 (t, ^{C-F}J=4.2 Hz), 133.91, 131.94, 129.66, 124.94 (t, ^{C-F}J=25.6 Hz), 123.05, 121.36 (t, ^{C-F}J=239.8 Hz), 115.59, 108.66 (t, ^{C-F}J=8.9 Hz), 56.59, 56.03, 53.42, 33.60, 25.62, 24.81 (t, ^{C-F}J=28.9 Hz), 11.31.

¹⁹F{¹H} NMR (471 MHz, CDCl₃) δ (ppm) −86.52, −86.58.

HRMS (ESI): calcd. for C₂₂H₂₃F₂NO₄Na [M+Na]⁺=426.1493, found [M+Na]⁺=426.1488.

### 1-(4-(1,1-difluoroethyl)-2,5-dimethoxyphenyl)butan-2-amine (50)

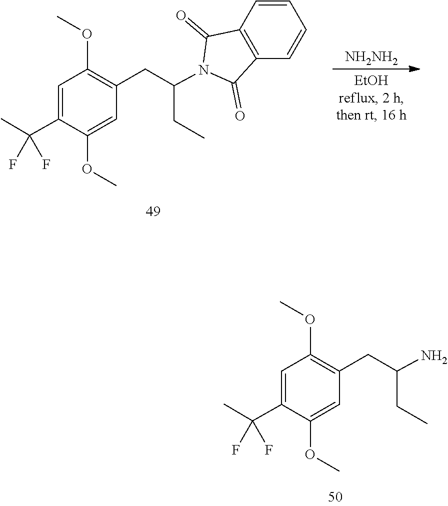

Hydrazine, 98% (249 μL, 7.932 mmol) was added dropwise to a solution of 2-(1-(4-(1,1-difluoroethyl)-2,5-dimethoxyphenyl)butan-2-yl)isoindoline-1,3-dione (320 mg, 0.793 mmol) in dry ethanol (10 mL) under argon atmosphere, and the resulting mixture was stirred at 100° C. for 2 hours. Then the reaction mixture was cooled to room temperature and stirred for 16 hours. The reaction mixture was filtered, washed with ethanol (20 mL), and the solvent was evaporated in vacuo. The obtained residue was suspended with dichloromethane (30 mL), filtered, and filtrate was concentrated in vacuo. The obtained residue suspended in water (30 mL) and extracted with ethyl acetate (3×100 mL). The combined organic extracts were washed with aqueous saturated NaHCO₃ solution (20 mL), brine solutions (20 mL), dried over MgSO₄, filtered, and the solvents were evaporated in vacuo. The residue obtained after the workup was purified by column chromatography (dichloromethane/methanol/7 M ammonia in methanol, 92:08:02). The product was obtained as a white solid (165 mg, 76%).

¹H NMR (500 MHz, CDCl₃) δ (ppm) 7.03 (s, 1H), 6.80 (s, 1H), 3.82 (s, 3H), 3.80 (s, 3H), 3.04 (br s, 1H), 2.86 (dd, J=13.3, 4.9 Hz, 1H), 2.76-2.37 (br s, 2H), 2.56 (dd, J=13.2, 8.2 Hz, 1H), 1.99 (t, J=18.7 Hz, 3H), 1.55 (tt, J=9.7, 4.4 Hz, 1H), 1.43 (dt, J=14.1, 7.3 Hz, 1H), 0.99 (t, J=7.4 Hz, 3H).

¹³C NMR (126 MHz, CDCl₃) δ (ppm) 151.57, 150.52 (t, ^{C-F}J=4.3 Hz), 130.55, 124.83 (t, ^{C-F}J=25.6 Hz), 121.44 (t, ^{C-F}J=239.8 Hz), 115.98, 108.89 (t, ^{C-F}J=9.0 Hz), 56.75, 56.10, 53.01, 38.26, 30.01, 24.87 (t, ^{C-F}J=28.9 Hz), 10.61.

¹⁹F{¹H} NMR (471 MHz, CDCl₃) δ (ppm) −86.31, −86.33.

¹⁹F NMR (471 MHz, CDCl₃) δ (ppm) −85.42 (qd, ^{F-H}J=18.8, 9.4 Hz).

HRMS (ESI): calcd. for C₁₄H₂₂F₂NO₂ [M+H]⁺=274.1619, found [M+H]⁺=274.1622.

### Synthesis of 1-(4-(1,1-difluoropropyl)-2,5-dimethoxyphenyl)butan-2-(53)

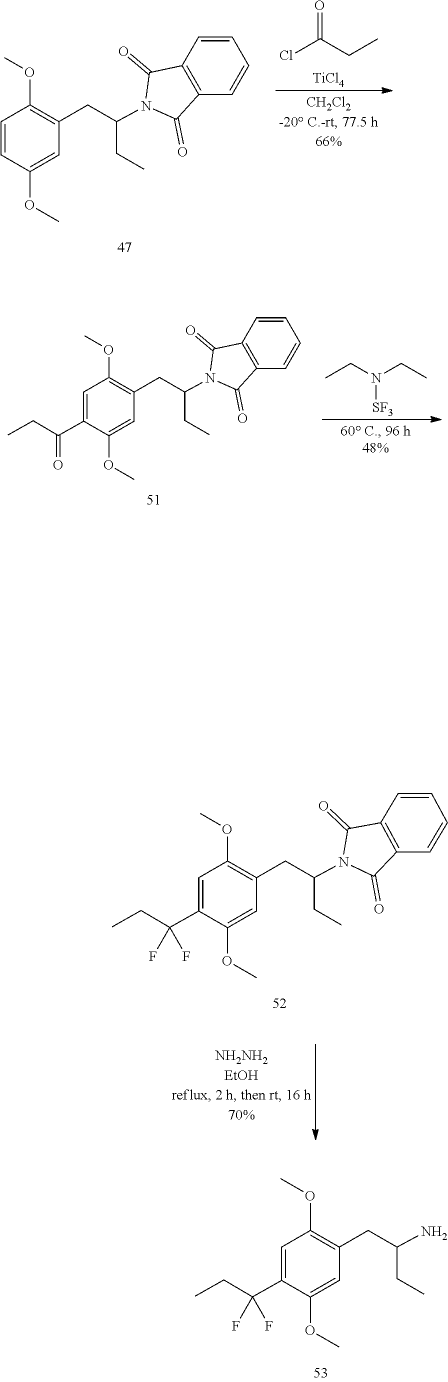

### 2-(1-(2,5-dimethoxy-4-propionylphenyl)butan-2-yl)isoindoline-1,3-dione (51)

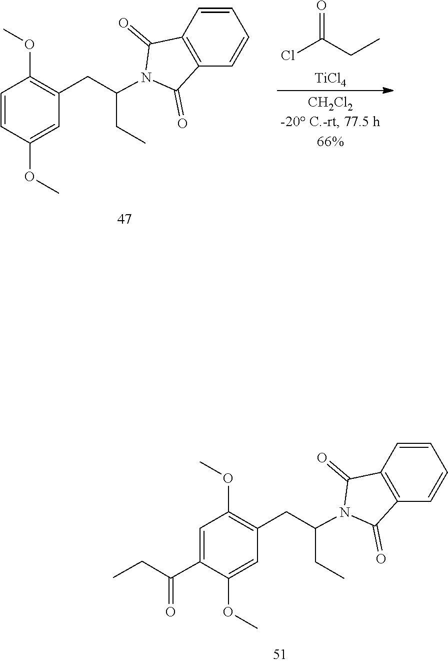

TiCl₄ (5.48 mL, 5.48 mmol; 1M in dichloromethane) was added dropwise to a cooled (−20° C.) solution of 2-(1-(2,5-dimethoxyphenyl)butan-2-yl)isoindoline-1,3-dione (1 g, 2.946 mmol) in dry dichloromethane (30 mL) under argon atmosphere. Then propionyl chloride (309 μL, 3.536 mmol) was added dropwise to the reaction mixture, and the resulting mixture was stirred at −20° C. for 30 minutes. Then the reaction mixture was warmed to room temperature and stirred for 77 hours. The reaction mixture was quenched with water (35 mL) and extracted with dichloromethane (2×100 mL). The combined organic extracts were dried over MgSO₄, filtered, and the solvent was evaporated in vacuo. The residue obtained after the workup was purified by column chromatography (hexane/ethyl acetate, 80:20). The product was obtained as a white solid (770 mg, 66%).

¹H NMR (500 MHz, CDCl₃) δ (ppm) 7.76-7.70 (m, 2H), 7.68-7.61 (m, 2H), 7.15 (s, 1H), 6.66 (s, 1H), 4.51 (tt, J=10.3, 5.2 Hz, 1H), 3.72 (s, 3H), 3.62 (s, 3H), 3.32 (dd, J=13.5, 10.3 Hz, 1H), 3.11 (dd, J=13.5, 5.1 Hz, 1H), 2.88 (q, J=7.3 Hz, 2H), 2.26-2.13 (m, 1H), 1.93-1.81 (m, 1H), 1.07 (t, J=7.3 Hz, 3H), 0.91 (t, J=7.4 Hz, 3H).

¹³C NMR (126 MHz, CDCl₃) δ (ppm) 202.32, 168.69, 152.99, 151.75, 133.88, 133.08, 131.89, 126.46, 123.03, 114.93, 111.69, 56.04, 55.86, 53.37, 37.09, 33.76, 25.69, 11.27, 8.53.

HRMS (ESI): calcd. for C₂₃H₂₅NO₅Na [M+Na]⁺=418.1631, found [M+Na]⁺=418.1638.

### 2-(1-(4-(1,1-difluoropropyl)-2,5-dimethoxyphenyl)butan-2-yl)isoindoline-1,3-dione (52)

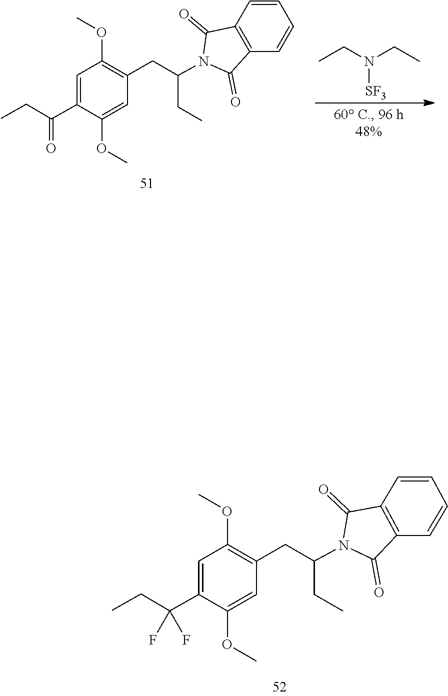

In a glass vial, diethylaminosulfur trifluoride (3.24 mL, 24.528 mmol) was added to 2-(1-(2,5-dimethoxy-4-propionylphenyl)butan-2-yl)isoindoline-1,3-dione (970 mg, 2.452 mmol) under argon atmosphere. The resulting reaction mixture was stirred at 60° C. for 96 hours. The rection mixture was carefully quenched with aqueous saturated NaHCO₃ solutions (60 mL) and extracted with ethyl acetate (3×60 mL). The combined organic extracts were dried over MgSO₄, filtered, and the solvent was evaporated in vacuo. The residue obtained after the workup was purified by column chromatography (hexane/ethyl acetate, 90:10). The product was obtained as a white solid (490 mg, 48%).

¹H NMR (500 MHz, CDCl₃) δ (ppm) 7.77-7.69 (m, 2H), 7.69-7.62 (m, 2H), 6.88 (s, 1H), 6.64 (s, 1H), 4.51 (tt, J=10.3, 5.3 Hz, 1H), 3.74 (s, 3H), 3.55 (s, 3H), 3.27 (dd, J=13.5, 10.2 Hz, 1H), 3.13 (dd, J=13.5, 5.3 Hz, 1H), 2.27-2.12 (m, 3H), 1.88 (dtd, J=14.0, 7.4, 5.4 Hz, 1H), 0.92 (t, J=7.4 Hz, 3H), 0.80 (t, J=7.5 Hz, 3H).

¹³C NMR (126 MHz, CDCl₃) δ (ppm) 168.75, 151.55, 150.29 (t, ^{C-F}J=4.3 Hz), 133.88, 131.93, 129.58, 123.86 (t, ^{C-F}J=25.7 Hz), 123.16 (t, ^{C-F}J=242.5 Hz), 123.00, 115.59, 109.46 (t, ^{C-F}J=Hz), 56.61, 56.03, 53.45, 33.63, 30.30 (t, ^{C-F}J=27.2 Hz), 25.59, 11.33, 7.07 (t, ^{C-F}J=5.0 Hz).

¹⁹F{¹H} NMR (471 MHz, CDCl₃) δ (ppm) −95.81, −95.86.

HRMS (ESI): calcd. for C₂₃H₂₅F₂NO₄Na [M+Na]⁺=440.1649, found [M+Na]⁺=440.1661.

### 1-(4-(1,1-difluoropropyl)-2,5-dimethoxyphenyl)butan-2-amine (53)

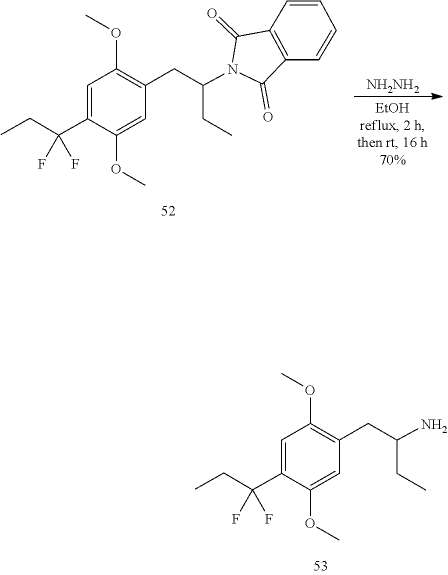

Hydrazine, 98% (331 μL, 10.540 mmol) was added dropwise to a solution of 2-(1-(4-(1,1-difluoropropyl)-2,5-dimethoxyphenyl)butan-2-yl)isoindoline-1,3-dione (440 mg, 1.054 mmol) in dry ethanol (15 mL) under argon atmosphere, and the resulting mixture was stirred at 100° C. for 2 hours. Then the reaction mixture was cooled to room temperature and stirred for 16 hours. The reaction mixture was filtered, washed with ethanol (20 mL), and the solvent was evaporated in vacuo. The obtained residue was suspended with dichloromethane (30 mL), filtered, and filtrate was concentrated in vacuo. The obtained residue suspended in water (30 mL) and extracted with ethyl acetate (3×100 mL). The combined organic extracts were washed with aqueous saturated NaHCO₃ solution (30 mL), brine solutions (25 mL), dried over MgSO₄, filtered, and the solvents were evaporated in vacuo. The residue obtained after the workup was purified by column chromatography (dichloromethane/methanol/7 M ammonia in methanol, 92:08:02). The product was obtained as a light brownish wax (210 mg, 70%), which turned solid in freezer.

¹H NMR (500 MHz, CDCl₃) δ (ppm) 6.99 (s, 1H), 6.78 (s, 1H), 3.80 (s, 3H), 3.79 (s, 3H), 3.06-2.91 (m, 1H), 2.84 (d, J=12.7 Hz, 1H), 2.48 (dd, J=13.1, 8.3 Hz, 1H), 2.32 (ddt, J=17.1, 9.6, 7.5 Hz, 2H), 1.71-1.42 (m, 3H), 1.37 (dt, J=14.0, 7.2 Hz, 1H), 0.98 (t, J=7.4 Hz, 3H), 0.94 (t, J=7.5 Hz, 3H).

¹³C NMR (126 MHz, CDCl₃) δ (ppm) 151.59, 150.51 (t, ^{C-F}J=4.4 Hz), 131.00 (t, ^{C-F}J=1.6 Hz), 123.71 (t, ^{C-F}J=25.7 Hz), 123.21 (t, ^{C-F}J=242.5 Hz), 115.89, 109.65 (t, ^{C-F}J=9.2 Hz), 56.77, 56.11, 52.86, 38.91, 30.41 (t, ^{C-F}J=27.2 Hz), 29.82, 10.69, 7.11 (t, ^{C-F}J=5.0 Hz).

¹⁹F{¹H} NMR (471 MHz, CDCl₃) δ (ppm) −95.74, −95.86.

¹⁹F NMR (471 MHz, CDCl₃) δ (ppm) −94.85 (t, ^{F-H}J=16.9 Hz), −94.96 (t, ^{F-H}J=17.1 Hz).

HRMS (ESI): calcd. for C₁₅H₂₄F₂NO₂ [M+H]⁺=288.1775, found [M+H]⁺=288.1777.

### Synthetic Scheme for 1-(4-(2,2-difluoropropyl)-2,5-dimethoxyphenyl)butan-2-amine (58)

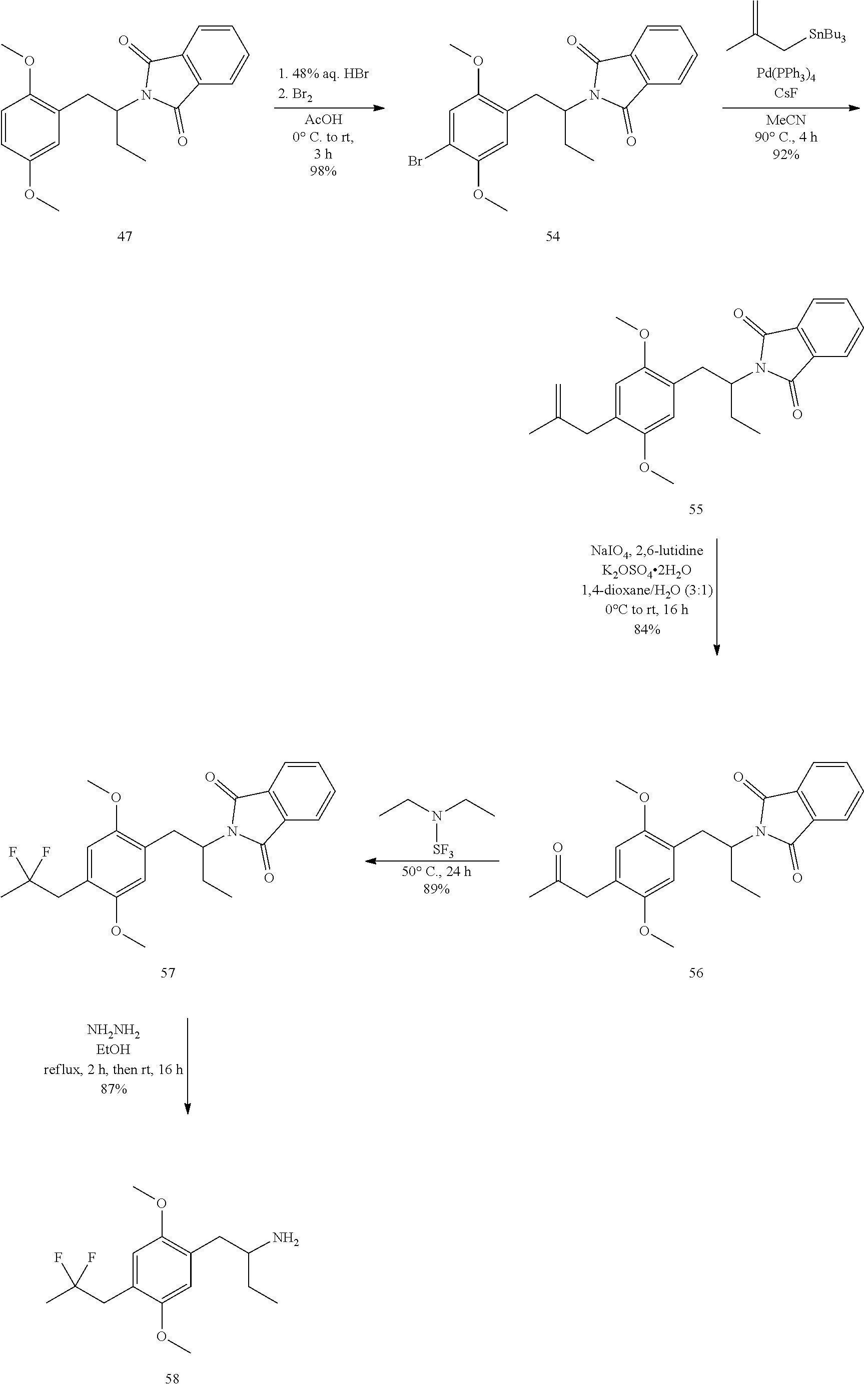

### 2-(1-(4-bromo-2,5-dimethoxyphenyl)butan-2-yl)isoindoline-1,3-dione (54)

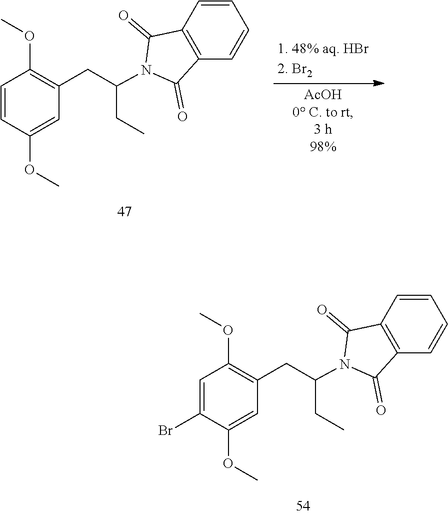 [SMILES: CCC(Cc1cc(OC)ccc1OC)N1C(=O)c2ccccc2C1=O ; CCC(Cc1cc(OC)c(Br)cc1OC)N1C(=O)c2ccccc2C1=O]

A solution of 48% aqueous HBr (632 μL, 11.639 mmol) in acetic acid (6 mL) was added dropwise to a cooled (0° C.) solution of 2-(1-(2,5-dimethoxyphenyl)butan-2-yl)isoindoline-1,3-dione (1.58 g, 4.655 mmol) in acetic acid (10 mL) under argon atmosphere. Then a solution of bromine (250 μL, 4.888 mmol) in acetic acid (10 mL) was added dropwise to the reaction mixture at 0° C., and the resulting mixture was warmed to room temperature and stirred for 3 hours. The reaction mixture was concentrated in vacuo. The obtained residue was quenched with aqueous saturated NaHCO₃ solutions (60 mL) and extracted with ethyl acetate (80 mL×2). The combined organic extracts were dried over MgSO₄, filtered, and the solvent was evaporated in vacuo. The product was obtained as a colorless wax (1.90 g, 98%). The obtained product was used into the next step without purification.

¹H NMR (500 MHz, CDCl₃) δ (ppm) 7.79-7.71 (m, 2H), 7.69-7.63 (m, 2H), 6.93 (s, 1H), 6.59 (s, 1H), 4.48 (tt, J=10.6, 5.2 Hz, 1H), 3.72 (s, 3H), 3.58 (s, 3H), 3.24 (dd, J=13.6, 10.4 Hz, 1H), 3.08 (dd, J=13.7, 5.1 Hz, 1H), 2.18 (dp, J=21.9, 7.7 Hz, 1H), 1.86 (dp, J=13.9, 6.8 Hz, 1H), 0.91 (t, J=7.5 Hz, 3H).

¹³C NMR (126 MHz, CDCl₃) δ (ppm) 168.77, 152.25, 149.72, 133.93, 131.84, 127.23, 123.07, 115.91, 115.11, 109.47, 56.88, 56.17, 53.35, 33.55, 25.61, 11.30.

HRMS (ESI): calcd. for C₂₀H₂₀BrNO₄Na [M+Na]⁺=442.0456, found [M+Na]⁺=442.0452.

### 2-(1-(2,5-dimethoxy-4-(2-methylallyl)phenyl)butan-2-yl)isoindoline-1,3-dione (55)

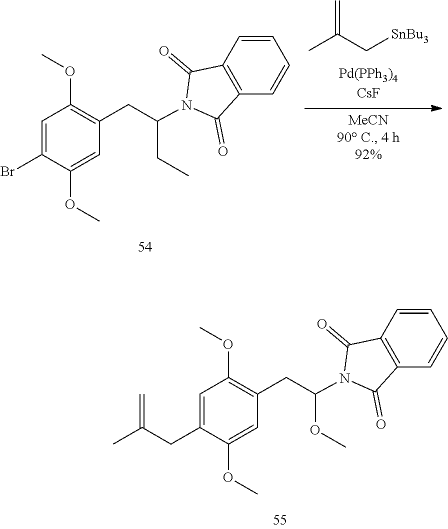

In glass vial, cesium fluoride (1.48 g, 9.754 mmol), tributyl(2-methylallyl)stannane (1.35 g, 3.902 mmol) and tetrakis(triphenylphosphine)palladium(0) (376 mg; 0.325 mmol) were added to a solution of 2-(1-(4-bromo-2,5-dimethoxyphenyl)butan-2-yl)isoindoline-1,3-dione (1.36 g, 3.251 mmol) in acetonitrile (25 mL), and the resulting mixture was stirred at 90° C. for 4 hours. The reaction mixture was cooled to 25° C., filtered through a pad of Celite and washed with dichloromethane (80 mL). The filtrate was concentrated in vacuo. The residue obtained after the workup was purified by column chromatography (hexane/ethyl acetate, 90:10). The product was obtained as a pale-yellow wax (1.45 g, 92%).

¹H NMR (500 MHz, CDCl₃) δ (ppm) 7.75-7.68 (m, 2H), 7.66-7.59 (m, 2H), 6.53 (s, 1H), 6.52 (s, 1H), 4.68 (s, 1H), 4.49 (tt, J=10.4, 5.4 Hz, 1H), 4.45 (s, 1H), 3.70 (s, 3H), 3.48 (s, 3H), 3.23-3.16 (m, 3H), 3.12 (dd, J=13.5, 5.4 Hz, 1H), 2.28-2.15 (m, 1H), 1.94-1.82 (m, 1H), 1.61 (s, 3H), 0.92 (t, J=7.4 Hz, 3H).

¹³C NMR (126 MHz, CDCl₃) δ (ppm) 168.76, 151.74, 151.21, 145.10, 133.71, 131.98, 127.18, 125.36, 122.87, 114.31, 113.17, 111.04, 56.31, 55.94, 53.86, 37.76, 33.61, 25.45, 22.47, 11.36.

HRMS (ESI): calcd. for C₂₄H₂₇NO₄Na [M+Na]⁺=416.1838, found [M+Na]⁺=416.1840.

### 2-(1-(2,5-dimethoxy-4-(2-oxopropyl)phenyl)butan-2-yl)isoindoline-1,3-dione (56)

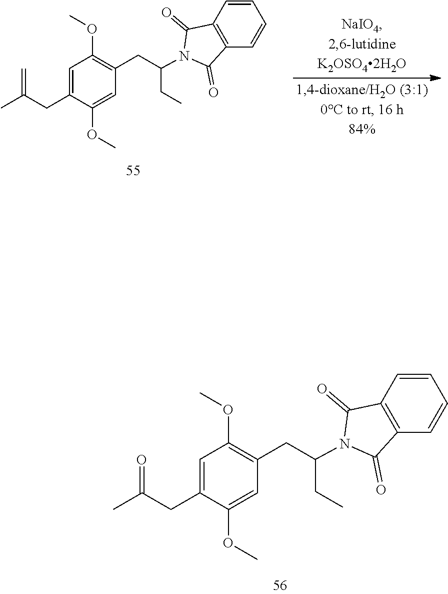 [SMILES: C=C(C)Cc1cc(OC)c(CC(CC)N2C(=O)c3ccccc3C2=O)cc1OC ; CCC(Cc1cc(OC)c(CC(C)=O)cc1OC)N1C(=O)c2ccccc2C1=O]

Sodium periodate (2.51 g, 11.721 mmol), 2,6 lutidine (1.021 mL, 8.768 mmol) and potassium osmate(VI) dihydrate (54 mg, 0.146 mmol) were added to a cooled (0° C.) solution of 2-(1-(2,5-dimethoxy-4-(2-methylallyl)phenyl)butan-2-yl)isoindoline-1,3-dione (1.153 g, 2.930 mmol) in acetone: water (3:1, 45 mL:15 mL), and the resulting mixture was stirred at room temperature for 16 hours. The reaction mixture quenched with aqueous 5% sodium thiosulfate solutions (15 mL) and extracted with ethyl acetate (80 mL×3). The combined organic extracts were dried over MgSO₄, filtered, and the solvent was evaporated in vacuo. The residue obtained after the workup was purified by column chromatography (hexane/ethyl acetate, 80:20). The product was obtained as a pale-yellow wax (970 mg, 84%).

¹H NMR (500 MHz, CDCl₃) δ (ppm) 7.75-7.68 (m, 2H), 7.67-7.61 (m, 2H), 6.55 (s, 1H), 6.53 (s, 1H), 4.47 (tt, J=10.2, 5.3 Hz, 1H), 3.69 (s, 3H), 3.51 (s, 2H), 3.49 (s, 3H), 3.21 (dd, J=13.5, 10.1 Hz, 1H), 3.11 (dd, J=13.5, 5.3 Hz, 1H), 2.27-2.14 (m, 1H), 1.94 (s, 3H), 1.93-1.81 (m, 1H), 0.91 (t, J=7.4 Hz, 3H).

¹³C NMR (126 MHz, CDCl₃) δ (ppm) 207.30, 168.76, 151.73, 150.92, 133.79, 131.94, 126.75, 122.91, 122.31, 113.88, 113.62, 55.97, 55.89, 53.78, 45.59, 33.65, 28.85, 25.46, 11.33.

HRMS (ESI): calcd. for C₂₃H₂₅NO₅Na [M+Na]⁺=418.1631, found [M+Na]⁺=418.1628.

### 2-(1-(4-(2,2-difluoropropyl)-2,5-dimethoxyphenyl)butan-2-yl)isoindoline-1,3-dione (57)

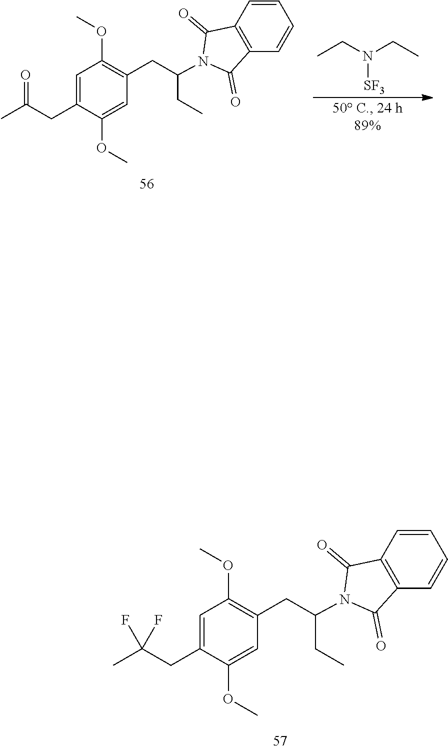

In a glass vial, diethylaminosulfur trifluoride (2.67 mL, 20.230 mmol) was added to 2-(1-(2,5-dimethoxy-4-(2-oxopropyl)phenyl)butan-2-yl)isoindoline-1,3-dione (800 mg, 2.023 mmol) under argon atmosphere. The resulting reaction mixture was stirred at 50° C. for 18 hours. The rection mixture was carefully quenched with aqueous saturated NaHCO₃ solutions (50 mL) and extracted with ethyl acetate (3×80 mL). The combined organic extracts were dried over MgSO₄, filtered, and the solvent was evaporated in vacuo. The residue obtained after the workup was purified by column chromatography (hexane/ethyl acetate, 90:10). The product was obtained as a pale-yellow solid (750 mg, 89%).

¹H NMR (500 MHz, CDCl₃) δ (ppm) 7.76-7.69 (m, 2H), 7.68-7.61 (m, 2H), 6.63 (s, 1H), 6.55 (s, 1H), 4.48 (tt, J=10.3, 5.3 Hz, 1H), 3.70 (s, 3H), 3.51 (s, 3H), 3.22 (dd, J=13.5, 10.1 Hz, 1H), 3.14-3.03 (m, 3H), 2.27-2.16 (m, 1H), 1.94-1.82 (m, 1H), 1.34 (t, J=18.6 Hz, 3H), 0.92 (t, J=7.4 Hz, 3H).

¹³C NMR (126 MHz, CDCl₃) δ (ppm) 168.77, 151.59, 151.36, 133.78, 131.98, 126.96, 124.19 (t, ^{C-F}J=239.8 Hz), 122.93, 121.19 (t, ^{C-F}J=5.5 Hz), 114.40, 114.01, 56.03, 55.95, 53.83, 37.34 (t, ^{C-F}J=27.0 Hz), 33.67, 25.49, 22.35 (t, ^{C-F}J=27.3 Hz), 11.37.

¹⁹F{¹H} NMR (471 MHz, CDCl₃) δ (ppm) −87.56, −87.64.

¹⁹F NMR (471 MHz, CDCl₃) δ (ppm) −86.68 (tt, ^{F-H}J=34.2, 16.9 Hz).

HRMS (ESI): calcd. for C₂₃H₂₅F₂NO₄Na [M+Na]⁺=440.1649, found [M+Na]⁺=440.1646.

### 1-(4-(2,2-difluoropropyl)-2,5-dimethoxyphenyl)butan-2-amine (58)

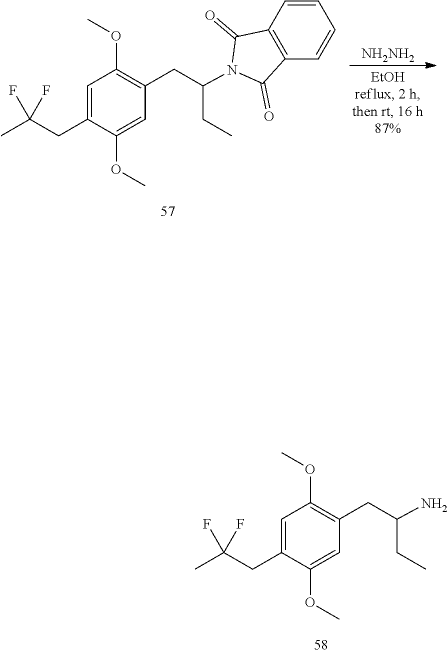

Hydrazine, 98% (526 μL, 16.768 mmol) was added dropwise to a solution of 2-(1-(4-(2,2-difluoropropyl)-2,5-dimethoxyphenyl)butan-2-yl)isoindoline-1,3-dione (700 mg, 1.677 mmol) in dry ethanol (15 mL) under argon atmosphere, and the resulting mixture was stirred at 100° C. for 2 hours. Then the reaction mixture was cooled to room temperature and stirred for 16 hours. The reaction mixture was filtered, washed with ethanol (60 mL), and the solvent was evaporated in vacuo. The obtained residue suspended in water (35 mL) and extracted with ethyl acetate (3×90 mL). The combined organic extracts were washed with aqueous saturated NaHCO₃ solution (20 mL), brine solutions (20 mL), dried over MgSO₄, filtered, and the solvents were evaporated in vacuo. The residue obtained after the workup was purified by column chromatography (dichloromethane/methanol/7 M ammonia in methanol, 90:10:02). The product was obtained as a white solid (420 mg, 87%).

¹H NMR (500 MHz, CDCl₃) δ (ppm) 6.76 (s, 1H), 6.71 (s, 1H), 3.78 (s, 3H), 3.76 (s, 3H), 3.18 (t, J=15.5 Hz, 2H), 3.00-2.93 (m, 1H), 2.81 (dd, J=13.2, 4.7 Hz, 1H), 2.47 (dd, J=13.2, 8.5 Hz, 1H), 1.81 (br s, 2H), 1.56-1.49 (m, 1H), 1.51 (t, J=18.5 Hz, 3H), 1.38 (dt, J=13.6, 7.3 Hz, 1H), 0.98 (t, J=7.4 Hz, 3H).

¹³C NMR (126 MHz, CDCl₃) δ (ppm) 151.67, 151.64, 128.18, 124.20 (t, ^{C-F}J=239.4 Hz), 120.86 (t, ^{C-F}J=5.2 Hz), 114.77, 114.35, 56.27, 56.06, 53.05, 38.74, 37.43 (t, ^{C-F}J=26.9 Hz), 30.43, 22.73 (t, ^{C-F}J=27.4 Hz), 10.69.

¹⁹F{¹H} NMR (471 MHz, CDCl₃) δ (ppm) −87.80.

¹⁹F NMR (471 MHz, CDCl₃) δ (ppm) −86.90 (dtd, ^{F-H}J=34.1, 18.5, 15.4 Hz).

HRMS (ESI): calcd. for C₁₅H₂₄F₂NO₂ [M+H]⁺=288.1775, found [M+H]⁺=288.1781.

### Example 8 Synthesis of Racemic 1-(4-(fluoroalkyl)-2,5-dimethoxyphenyl)butan-2-amines

### 1-(4-(1-fluoroethyl)-2,5-dimethoxyphenyl)butan-2-amine

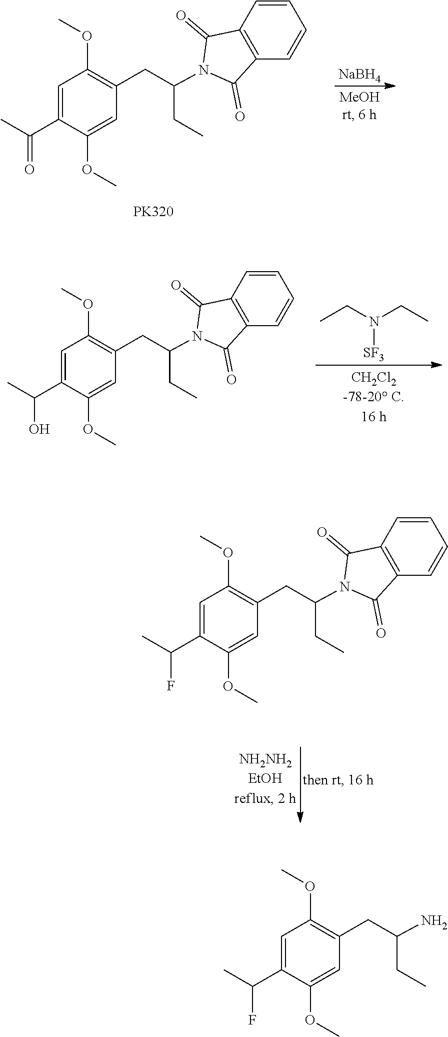

### 1-(4-(2-fluoropropyl)-2,5-dimethoxyphenyl)butan-2-amine

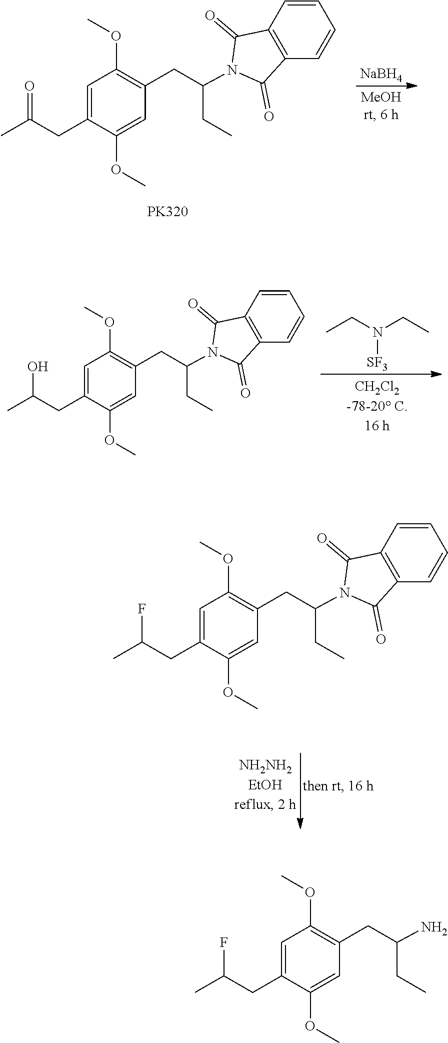

Synthesis of racemic 1-(4-(fluoroalkyl)-2,5-dimethoxyphenyl)butan-2-amines is achieved via borohydride reduction of ketone to the alcohol, followed by monofluorination with diethylaminosulfur trifluoride (DAST). Deprotection of the pthalimide protecting group with hydrazine affords the desired compounds as a mixture of diastereomers.

### Example 9 Separation of Enantiomers

Enantiomers of Ariadne were separated at WuXi Apptec (Shanghai) using the following methods. Compound was dissolved in 100 mL methanol/DCM and injected in 0.8 mL portions. Following separations, the fractions were dried off via rotary evaporator at bath temperature 40° C. Waters UPCC with PDA Detector was used for chiral SFC trace.

### Analytical Separation Method:

Instrument: Waters UPC2 analytical SFC (SFC-H)
Column: ChiralPak AD, 150×4.6 mm I.D., 3 μm
Mobile phase: A for CO₂ and B for isopropanol (0.05% diethylamine)
Gradient: B 5-40%
Flow rate: 2.4 mL/min
Back pressure: 100 bar
Column temperature: 35° C.
Wavelength: 220 nm

### Preparative Separation Method:

Instrument: MG H preparative SFC (SFC-14)
Column: ChiralPak AD, 250×30 mm I.D., 10 μm
Mobile phase: A for CO₂ and B for Isopropanol (0.1% NH₃H₂O)
Gradient: B 15%
Flow rate: 60 mL/min
Back pressure: 100 bar
Column temperature: 38° C.
Wavelength: 220 nm
Cycle time: ˜6 min

### Example 10 Ariadne is an Agonist of 5-HT2 Receptors

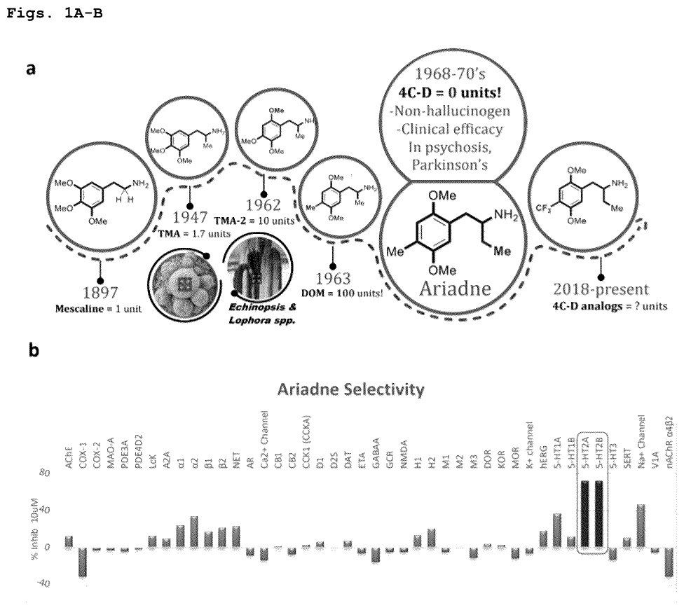

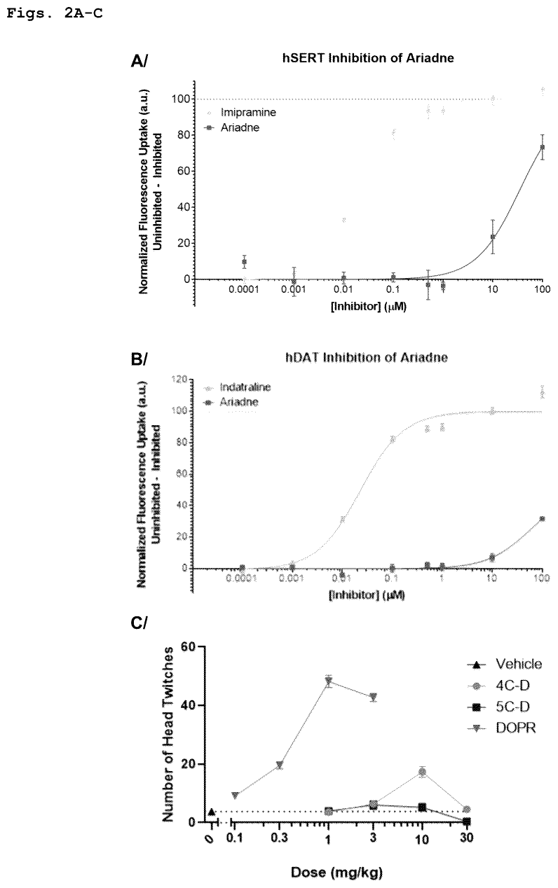

A broad screen of 44 molecular targets (SafetyScreen44, Eurofins-Panlabs) for Ariadne were performed, which includes G protein-coupled receptors, steroid nuclear receptors, ion channels, neurotransmitter transporters, and metabolic and signaling enzymes. Only two molecular targets, 5-HT_{2A} and 5-HT_{2B} receptors, emerged above the positive hit threshold, defined as 50% displacement of standard radioligands by 10 uM concentration of Ariadne (FIG. 1b, and Table 1). There was essentially no activity at the serotonin (SERT), dopamine (DAT) and norepinephrine (NET) transporters. The lack of SERT activity was confirmed in our laboratories in a SERT-mediated cell uptake assay, showing that Ariadne does not interact with SERT (FIG. S1)

### Eurofins Safety Screen 44 (Panlabs):

For species, “H”=human, “R”=rat. Test compound screened at all targets at concentration of 10 μM.

TABLE 1

| Assay | | % | Ligand or | Species, |
|---|---|---|---|---|
| ID | Target Name | Response | Substrate | Tissue |
| 104010 | Cholinesterase, | 12 | Acetylthiocholine | H, |
| | Acetyl, ACES | | | recombinant |
| 116030 | Cyclooxygenase | −31 | Arachidonic acid | H, |
| | COX-1 | | | recombinant |
| 118030 | Cyclooxygenase | −2 | Arachidonic Acid | H, |
| | COX-2 | | | recombinant |
| 140010 | Monoamine Oxidase | −3 | Kynuramine | H, |
| | MAO-A | | | recombinant |
| 152300 | Phosphodiesterase | −4 | FAM-cAMP | H, |
| | PDE3A | | | recombinant |
| 154420 | Phosphodiesterase | −2 | FAM-cAMP | H, |
| | PDE4D2 | | | recombinant |
| 176020 | Protein Tyrosine | 13 | Poly(Glu:Tyr) | H, |
| | Kinase, LCK | | | recombinant |
| 200610 | Adenosine A2A | 10 | [³H]CGS-21680 | H, |
| | | | | recombinant |
| 203110 | Adrenergic | 24 | [³H]Prazosin | H, |
| | alpha1A | | | recombinant |
| 203630 | Adrenergic | 34 | [³H]Rauwolscine | H, |
| | alpha2A | | | recombinant |
| 204010 | Adrenergic β1 | 17 | [¹²⁵I]Cyanopindolol | H, |
| | | | | recombinant |
| 204110 | Adrenergic β2 | 22 | [³H]CGP-12177 | H, |
| | | | | recombinant |
| 204410 | Transporter, | 23 | [¹²⁵I]RTI-55 | H, |
| | Norepinephrine | | | recombinant |
| | (NET) | | | |
| 206000 | Androgen | −8 | [³H]Methyltrienolone | H, |
| | (Testosterone) | | | LNCaP clone |
| | | | | FGC cells |
| 214600 | Calcium Channel | −13 | [³H]Nitrendipine | R, |
| | L-Type, | | | cerebral |
| | Dihydropyridine | | | cortex |
| 217050 | Cannabinoid CB1 | 1 | [³H]SR141716A | H, |
| | | | | recombinant |
| 217100 | Cannabinoid CB2 | −7 | [³H]WIN-55, 212-2 | H, |
| | | | | recombinant |
| 218030 | Cholecystokinin | 3 | [¹²⁵I]CCK-8 | H, |
| | CCK1 (CCKA) | | | recombinant |
| 219500 | Dopamine D1 | 7 | [³H]SCH-23390 | H, |
| | | | | recombinant |
| 219700 | Dopamine D2S | 1 | [³H]Spiperone | H, |
| | | | | recombinant |
| 220320 | Transporter, | 7 | [¹²⁵I]RTI-55 | H, |
| | Dopamine (DAT) | | | recombinant |
| 224010 | Endothelin ETA | −6 | [¹²⁵I]Endothelin-1 | H, |
| | | | | recombinant |
| 226600 | GABAA, | −15 | [³H]Flunitrazepam | R, |
| | Flunitrazepam, | | | brain |
| | Central | | | (minus |
| | | | | cerebellum) |
| 232030 | Glucocorticoid | −5 | [³H]Dexamethasone | H, |
| | | | | recombinant |
| 232810 | Glutamate, NMDA, | −5 | [³H]CGP-39653 | R, |
| | Agonism | | | cerebral |
| | | | | cortex |
| 239610 | Histamine H1 | 13 | [³H]Pyrilamine | H, |
| | | | | recombinant |
| 239710 | Histamine H2 | 21 | [¹²⁵I] | H, |
| | | | Aminopotentidine | recombinant |
| 252610 | Muscarinic M1 | −4 | [³H]N- | H, |
| | | | Methylscopolamine | recombinant |
| 252710 | Muscarinic M2 | 0 | [³H]N- | H, |
| | | | Methylscopolamine | recombinant |
| 252810 | Muscarinic M3 | −11 | [³H]N- | H, |
| | | | Methylscopolamine | recombinant |
| 260130 | Opiate δ1 (OP1, | 4 | [³H]Naltrindole | H, |
| | DOP) | | | recombinant |
| 260210 | Opiate kappa | 3 | [³H]Diprenorphine | H, |
| | (OP2, KOP) | | | recombinant |
| 260410 | Opiate μ (OP3, | −11 | [³H]Diprenorphine | H, |
| | MOP) | | | recombinant |
| 265510 | Potassium Channel | −6 | [¹²⁵I]alpha- | R, |
| | [KA] | | Dendrotoxin | cerebral |
| | | | | cortex |
| 265910 | Potassium Channel | 18 | [³H]Dofetilide | H, |
| | hERG, | | | recombinant |
| | [³H]Dofetilide | | | |
| 271110 | Serotonin (5- | 36 | [³H]8-OH-DPAT | H, |
| | Hydroxytryptamine) | | | recombinant |
| | 5-HT1A | | | |
| 271230 | Serotonin (5- | 12 | [³H]GR125743 | H, |
| | Hydroxytryptamine) | | | recombinant |
| | 5-HT1B | | | |
| 271650 | Serotonin (5- | 72 | [³H]Ketanserin | H, |
| | Hydroxytryptamine) | | | recombinant |
| | 5-HT2A | | | |
| 271700 | Serotonin (5- | 72 | [³H]Lysergic | H, |
| | Hydroxytryptamine) | | acid diethylamide | recombinant |
| | 5-HT2B | | (LSD) | |
| 271910 | Serotonin (5- | −13 | [³H]GR-65630 | H, |
| | Hydroxytryptamine) | | | recombinant |
| | 5-HT3 | | | |
| 274030 | Transporter, | 11 | [³H]Paroxetine | H, |
| | Serotonin (5- | | | recombinant |
| | Hydroxytryptamine) | | | |
| | (SERT) | | | |
| 279510 | Sodium Channel, | 47 | [³H] | H, |
| | Site 2 | | Batrachotoxinin | recombinant |
| 287530 | Vasopressin V1A | −5 | [¹²⁵I]Phenylacetyl | H, |
| | | | Tyr(Me)PheGlnAsn | recombinant |
| | | | ArgProArgTyr | |
| 299031 | Nicotinic | −31 | [³H]Cytisine | H, |
| | Acetylcholine | | | recombinant |
| | alpha4beta2, | | | |
| | Cytisine | | | |

Pharmacological exploration of Ariadne was performed with a screen of 44 molecular targets (SafetyScreen44, Eurofins-Panlabs), which includes G protein-coupled receptors, steroid nuclear receptors, ion channels, neurotransmitter transporters, and metabolic and signaling enzymes. Only two molecular targets, 5-HT_{2A} and 5-HT_{2B} receptors, emerged above the positive hit threshold, defined as 50% displacement of standard radioligands by 10 uM concentration of Ariadne. There was essentially no activity at the serotonin (SERT), dopamine (DAT) and norepinephrine (NET) transporters. The lack of SERT activity was confirmed in our laboratories in a SERT-mediated cell uptake assay, showing that Ariadne does not interact with SERT.

TABLE 2

| # | “4C-X” code | 5-HT2a | 5-HT2b |
|---|---|---|---|
| 32 | 4C-D | 1.3E−06M | >3.2E−05M |
| 35 | 4C-E | 1.9E−06M | 5.7E−07M |
| 42 | 4C-PR | 2.1E−07M | N.C. |
| 37 | 4C-MOM | 3.8E−06M | N.C. |
| 33 | 4C-Tone | 3.4E−06M | 2.5E−05M |
| 36 | 5-EtO-4C-TFM | 2.8E−07M | 9.5E−07M |
| 29 | 4C-TFM | 5.0E−08M | 6.3E−07M |
| 28 | 4C-T | 1.6E−07M | 6.6E−08M |
| 27 | 4C-T-2 | 5.3E−08M | 6.3E−07M |
| 41 | 4C-C | 1.9E−07M | 1.4E−05M |
| 31 | 4C-B | 9.0E−08M | 5.5E−06M |
| 34 | 4C-I | 9.7E−06M | 1.8E−05M |
| 39 | 5C-D | N.C. | 2.3E−05M |
| 38 | 4C(cycPr)-D | 9.7E−06M | 2.1E−05M |
| 40 | 4C-psiD | 1.0E−05M | N.C. |
| 30 | 4C-CycPR | 3.7E−06M | 3.2E−05M |
| 4 | 4C-CN | 1.1E−05M | N.C. |

5-HT2A and 5-HT2B IP1 agonism summary (Duplicate averages only), performed at Cerep, Eurofins

### Example 11 GPCR G Protein-Dissociation and β-Arresting Recruitment BRET Assays

### Results

Racemic and (R)-Ariadne were profiled in a functional assay panel of 12 human 5-HT receptors using a G protein activation BRET assay (“5-HTome” screen, FIGS. 2A-B). Robust agonist activity at 5-HT_{2A/2B/2C} receptors was confirmed (Eₘₐₓ>70%, EC₅₀<1 uM) for racemic and (R)-Ariadne (FIG. 2C). Little to no agonist activity for either compound was detected at 5-HT₄, 5-HT₅ₐ, 5-HT₆ and 5-HT₇ₐ receptors. At 5-HT₁ subtypes, most activity was very weak (Eₘₐₓ<70%, EC₅₀>10 uM), with the exception at 5-HT₁E (EC₅₀˜0.2 uM, (R)-Ariadne) and 5-HT_{1F} (EC₅₀˜5 uM, (R)-Ariadne) receptors where activity was detected at micromolar concentrations in the higher efficacy range (Eₘₐₓ>70%).

To further examine the 5-HT2 receptor signaling induced by Ariadne, we performed both the Gq dissociation BRET and calcium flux assays (FIG. 2C) on the racemate and isolated enantiomers. Ariadne showed more potent agonist activity at 5-HT_{2A} (EC₅₀=149 nM, (R)-Ariadne), versus 5-HT_{2B} (EC₅₀=739 nM, (R)-Ariadne), and 5-HT_{2C} receptors (EC₅₀=249 nM, (R)-Ariadne). Racemic Ariadne showed similar dose-dependent curves with a small but consistent right-shift compared to (R)-Ariadne, serving as an internal control and indicating greater potency of the R—versus S-enantiomers (see below). Noteworthy is that racemate/enantiomers of Ariadne exhibited partial agonism versus 5-HT (Eₘₐₓ=75% relative to 5-HT, (R)-Ariadne). Therefore, an orthogonal assay measuring calcium flux was also performed for the 5-HT₂ receptors (Klein et al., 2020). A similar range of potency, to that observed in Gq dissociation assay, was found at 5-HT_{2A} receptor (EC₅₀=30 nM, Eₘₐₓ=96% relative to 5-HT, (R)-Ariadne), (FIG. 2c). A decrease in calcium signaling efficacy was measured at both the 5-HT_{2B} receptor (Eₘₐₓ=64% relative to 5-HT, FIG. 2c), and 5-HT_{2C} receptor (Eₘₐₓ=60% relative to 5-HT). For the latter receptor, the low efficacy of calcium signaling is likely related to different kinetics of 5-HT_{2C} activation relative to that of 5-HT_{2A/2B} receptors, which calcium signaling assays are not adept at capturing at G_{q}-coupled GPCRs (Bdioui et al., 2018). The estimates of binding affinity at human 5-HT_{2A} receptor were obtained via a radioligand displacement assay (standard radioligand, ^{[251]}I-DOI) for racemic Ariadne (Ki=120 nM), (R)-Ariadne (Kᵢ=53 nM), and (S)-Ariadne (Kᵢ=220 nM). The affinity values obtained from an agonist radioligand are consistent with the range of functional potencies determined in the 5-HT_{2A} receptor signaling assays.

All BRET assays were conducted using BRET2 in HEK293T cells (ATCC CRL-11268; mycoplasma-free), which were subcultured in high-glucose DMEM (VWR) supplemented with 10% FBS (Life Technologies).

Constructs in G protein-dissociation BRET assays were derived from the codon-optimized Tango pcDNA3.1 library (Addgene) with V2tail/TEV/tTA encoding regions deleted to yield “de-Tango” constructs. 5-HT receptor constructs used in β-Arrestin2 recruitment BRET assays were also derived from the Tango library with V2tail/TEV/tTA encoding regions replaced with Renilla luciferase (Rluc8) using Gibson Assembly. All Gα-Rluc8, Beta, and GFP2-γ constructs were derived from the TRUPATH pcDNA5/FRT/TO library (Addgene). Geneblocks with N-terminal GFP2-fused human β-Arrestin2 constructs were synthesized by Integrated DNA Technologies (IDT) and subcloned into pcDNA3.1.

Approximately 48 hours before assays, cells were transfected using a reverse transfection method and plated in 1% dFBS at an approximate density of 15,000 cells per well into poly-L-lysine-coated 384-well white assay plates (Grenier Bio-One). For G protein-dissociation assays, cells were transfected in a 1:1:1:1 ratio of receptor: Gα-Rluc8: Beta: GFP2-γ constructs. For β-Arrestin2 recruitment assays, cells were transfected a 1:7.5 ratio of 5-HT-Rluc8:GFP2-fused human β-Arrestin2. All transfections were prepared in Opti-MEM (Invitrogen) and used a 3:1 ratio of TransIT-2020 (Mirus) uL:ug total DNA.

On the day of the assay, plates were decanted and 20 uL of drug buffer per well (1×HBSS, 20 mM HEPES, pH 7.4) was added using a Multidrop (ThermoFisher Scientific), and plates were allowed to equilibrate at 37° C. in a humidified incubator before receiving drug stimulation. Drug dilutions of all compounds were performed in McCorvy buffer (1×HBSS, 20 mM HEPES, pH 7.4, supplemented with 0.3% BSA fatty acid free (GoldBio), and 0.03% ascorbic acid). Drugs were dispensed using a FLIPRTETRA (Molecular Devices). Next, plates were incubated at 37° C. in a humidified incubator for 60 minutes or specified time point. Before reading, addition of coelenterazine 400a (5 uM final concentration; Nanolight Technology) was performed by the FLIPRTETRA. Immediately after, plates were read at 400 nm Rluc and fluorescent GFP2 emission at 510 nm for BRET2 at 0.8 second per well using a PheraStarFSX (BMB Lab Tech).

The BRET ratios of 510/400 luminescence were calculated per well and were plotted as a function of drug concentration using Graphpad Prism 5 or 9 (Graphpad Software Inc., San Diego, CA). Data were normalized to % positive control stimulation and analyzed using nonlinear regression “log(agonist) vs. response” to yield Emax and EC50 parameter estimates.

### Example 12 the Effect of 4-Position Substituent on 5-HT_{2A/2B/2C} Signaling

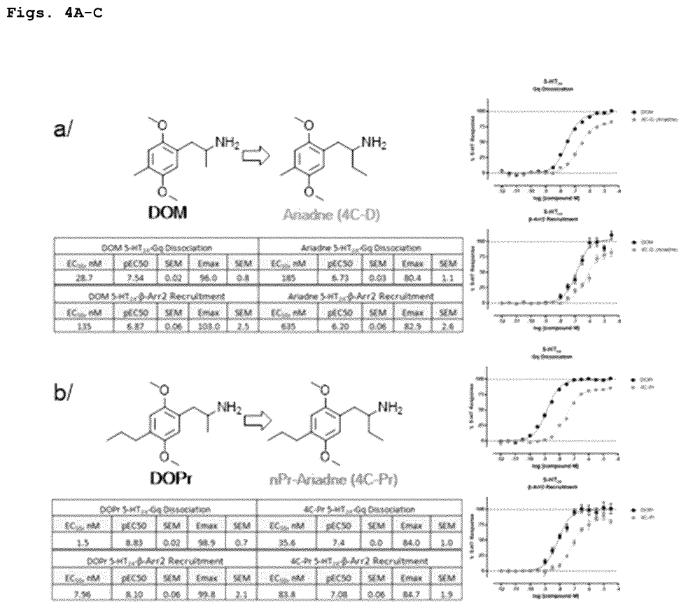

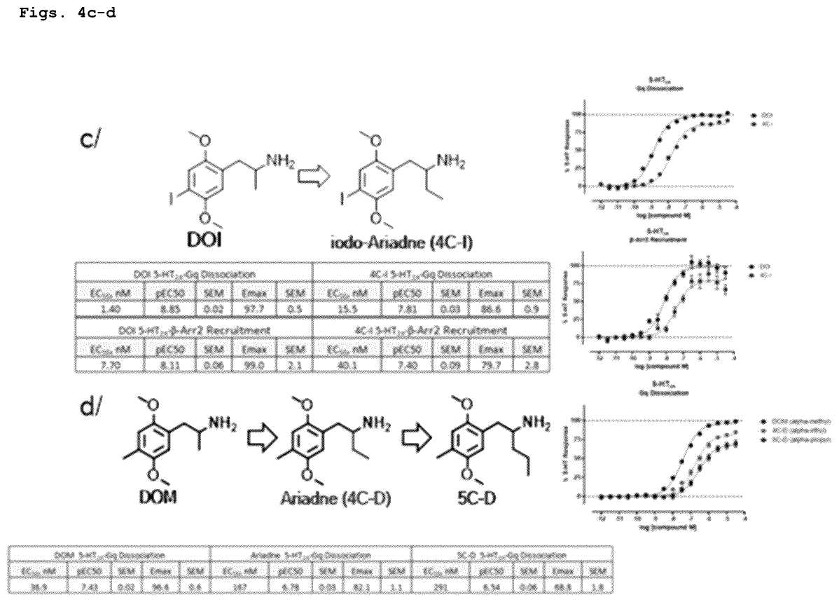

Considering the known SAR of the 4-position in the 2C-X and DOx systems (Nichols et al., 2017), we next explored the 4-position in the Ariadne series. We show that increasing the size and hydrophobicity of the 4-position led to an increase in the signaling potency at 5-HT_{2A} receptor. For example, comparison of Ariadne (EC_{50/Gq}=185 nM) to iodo-Ariadne (EC_{50/Gq}=16 nM) leads to a >10-fold increase in potency (FIG. 4). Similarly, there is a 20- to 30-fold increase in 5-HT_{2A} G_{q} dissociation potency between DOM and DOPR or DOI (FIG. 4), confirming the SAR trend in the 4-position across and Ariadne series.

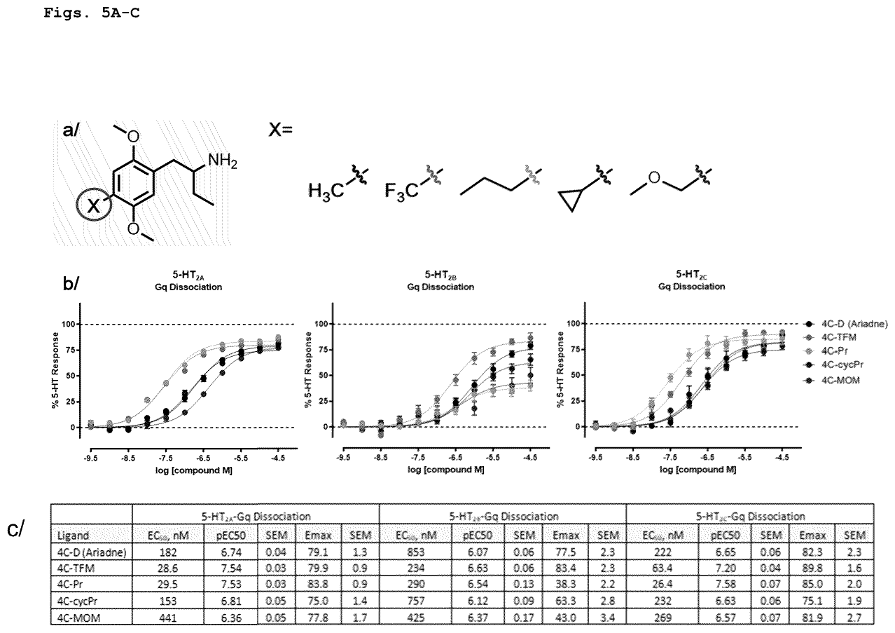

We were also interested in examining the selectivity for 5-HT_{2A} over 5-HT_{2B} receptors, as the latter is a molecular target linked to adverse cardiac effects (Rothman et al., 2009). We therefore synthesized a novel analog, TFM-Ariadne (4C-TFM), as a CF₃ group is known to render a potent analog in the DOx series, DOTFM (Nichols et al., 1994). Indeed, TFM-Ariadne was potent at 5-HT_{2A} receptor, comparable to iodo- and nPr-Ariadne (FIG. 5), but showed reduced signaling efficacy in G_{q} signaling assay (compared to 5-HT) in line with the other Ariadne analogs (FIG. 5). Notably, this compound shows 8-fold selectivity for 5-HT_{2A} over 5-HT_{2B} as measured by G_{q} dissociation signaling potency. We also synthesized a novel analog with a methoxymethyl (MOM) group in the 4-position, MOM-Ariadne (4C-MOM), examining substituents with increased polarity relative to the n-Pr group (Cunningham et al., 2023). As expected, this compound was approximately 2-fold less potent at 5-HT_{2A} compared to Ariadne, and twice as potent at 5-HT_{2B}, with similar potency maintained at 5-HT_{2C}. Similar signaling efficacies to Ariadne were found at 5-HT_{2A} (E_{max/Gq}=78%) and 5-HT_{2C} (E_{max/Gq}=82%) yet efficacy at 5-HT_{2B} diminished by nearly 2-fold (E_{max/Gq}=43%). The 4-cyclopropyl analog, cycPr-Ariadne (4C-cycPr), was also prepared and examined, showing similar signaling profile to Ariande.

### Example 13 Drug Preparation and Administration for Animal Studies

All samples were prepared the same day testing was performed. Solids were weighed into small vials and dissolved in USP grade 0.85% saline with addition of 2 molar equivalents of glacial acetic acid. Sonication and gentle heating were applied until complete dissolution. The compounds were subsequently filtered through 0.45 μm filters into a new glass vial. All compounds were administered at a 1 mg/kg subcutaneous dose at a volume of 10 mL/kg of body weight. Volinanserin hydrochloride salt (MDL 100907) was purchased from Toronto Research Chemicals.

### Example 14 General Mouse Use

All experimental procedures involving animals were approved by an Institutional Animal Care and Use Committee (IACUC) and adhered to principles described in the National Institutes of Health Guide for the Care and Use of Laboratory Animals. The studies were conducted at AAALAC accredited facilities. Animals received regular veterinary care (weekly by institutional veterinarians) including daily health monitoring (by experimenters) of the animals (observing home cage behaviors, nesting, and body weight). All procedures were designed to minimize any stress/distress. Healthy adult male mice C57BL/6J (8-12 weeks, 26-33 g) were purchased from the Jackson Laboratory (Bar Harbor, ME) and housed 5 mice per cage with food and water available ad libitum. Mice were maintained on a 12-h light/dark cycle (lights on 7:00-19:00) and all testing was done in the light cycle. Temperature was kept constant at 22±2° C., and relative humidity was maintained at 50±5%

### Example 15 Open Field and Head Twitch Response Behavioral Assays in Mice

### Open Field Locomotion Protocol

Mice were allowed to habituate for 30 min. Immediately after receiving a subcutaneous injection of the compound solution, mice were then placed gently in a clear Plexiglas arena (27.31×27.31×20.32 cm, Med Associates ENV-510) lit with ambient light (˜330 lux) and allowed to ambulate freely for 120 min. The locomotion of the animals was tracked by infrared beams embedded along the X, Y, Z axes of the area and automatically recorded. Data was collected on Activity Monitor by Med Associates. Simultaneously, HTR activity was recorded with a GoPro HERO9 at a 120 Hz.

### Head Twitch Response Protocol

The head-twitch response evaluation was performed by a trained observer who was blinded to both drug and dose. Mice were moved to the testing room 30 min before the experiment to allow for acclimation. The body weight of each mouse was recorded. Mice were administered either a 1, 3, 10, or 30 mg/kg s.c. dose of compound solution (volume of injection 260-330 μL based on body weight). After injection, mice were allowed to rest for 5 min and then were observed for 15 min during which the HTR (defined as a rapid rotational movement of the head around the longitudinal axis of the animals' body) was scored. The total number of head-twitches over the 15 minute period were recorded. Five mice were used per dose.

DOI was tested at the doses of 0.3, 1, 3, and 10 mg/kg (s.c., 5 mice per dose) and testing began immediately after administration of the compound. Ariadne was tested at the doses of 1, 3, 10, and 30 mg/kg (s.c., 5 mice per dose) and there was a 10 min interval between treatment and testing.

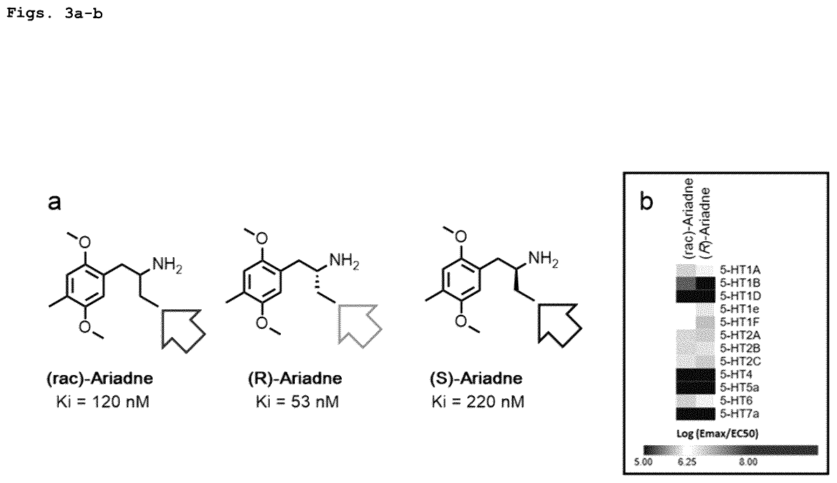

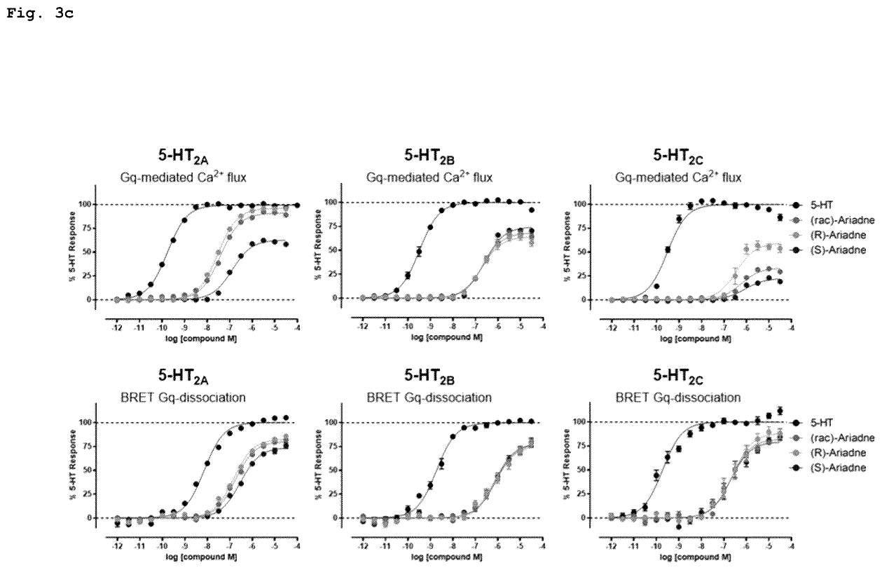

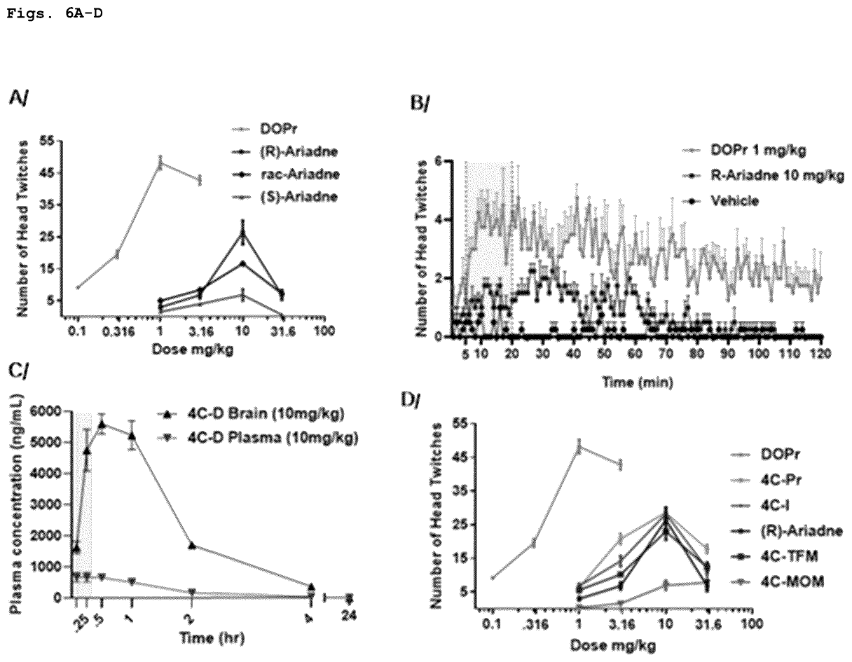

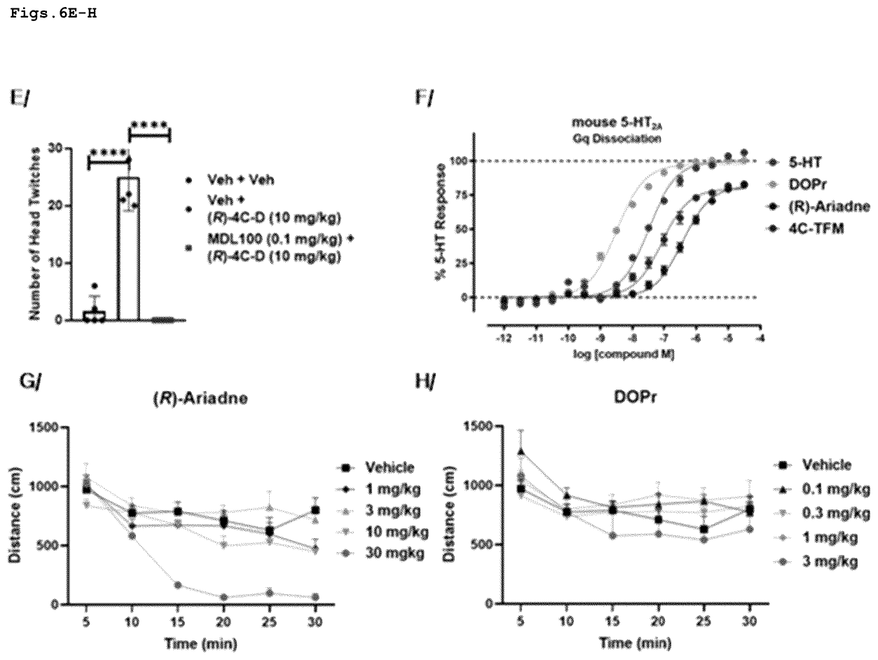

Ariadne and its analogs show attenuated head twitch behavior in mice, and effects in behavioral conflict tests. The head twitch response (HTR) in mice and rats is becoming a widely used preclinical readout for assessing the hallucinogenic potential of 5-HT_{2A} receptor agonists. It is characterized by intermittent twitches of rapid rotation of the head (Halberstadt et al., 2020). There is a strong correlation between the potency of psychedelic effects in humans and HTR in mice for a broad spectrum of psychedelic substances (Halberstadt et al., 2020) We used racemic DOPR as a reference phenylalkylamine psychedelic owing to its well-established hallucinogenic effects in humans, high potency agonism at 5-HT2A receptors in vitro and relatively simple pharmacological profile. DOPR showed a robust dose-dependent HTR response, peaking at 1 mg/kg (s.c. administration (FIG. 6D). The time profile revealed a rapid increase in HTR frequency reaching the maximum in 10 minutes post-drug administration, followed by a slow decay (FIG. 6B, the period used to determine dose-response is shown by the green band). Compared to racemic DOPR, racemic Ariadne showed a markedly attenuated HTR response (>3-fold decrease in HTR count per 15 min) with the maximum at a one-log higher dose (10 mg/kg, s.c., FIG. 6A). (rac)-Ariadne and the R- and S-enantiomers were numerically differentiated by the maximum number of head twitches in the order of their in vitro G_{q} protein signaling potency. Pharmacokinetic (PK) studies indicated high brain penetration by Ariadne (FIG. 6B-C) and showed that the HTR collection period occurs in the ascending portion of the PK curve. The Ariadne analogs with varying 4-position substitutions were also examined, showing a comparable dose-dependent response to that of (R)-Ariadne, and consistent with in vitro mouse 5-HT_{2A} potencies (G_{q}-BRET, FIG. 3C), with the exception of 4-methoxymethyl-Ariadne (4C-MOM) which elicited only residual HTR (FIG. 6F). Also, alpha-propyl-Ariadne (5C-D) induced HTR response that is indistinguishable from vehicle (FIG. 2C). The HTR of (R)-Ariadne and rac-trifluoromethyl-Ariadne was completely inhibited by MDL100,907 (volinanserin), a potent and selective 5-HT_{2A} receptor antagonist, demonstrating that the attenuated HTR of the Ariadne compound class is driven by 5-HT_{2A} activation in vivo (FIG. 6E). General locomotion in response to a novel environment (open field test, OF) showed a minor sedative effect at the peak HTR dose of (R)-Ariadne (10 mg/kg, s.c.), and a marked sedation at 30 mg/kg, consistent with previous studies in rats (Tilson et al., 1977). DOPR showed minor to no effects on locomotion, trending toward an increased locomotion at its HTR peak dose (1 mg/kg), also consistent with minor stimulatory effects of phenylalkylamine psychedelics in mice. These results indicate that there are no gross behavioral effects of Ariadne or DOPR in the ascending arm of the HTR dose range. Sedation becomes apparent in doses beyond the HTR maximum.

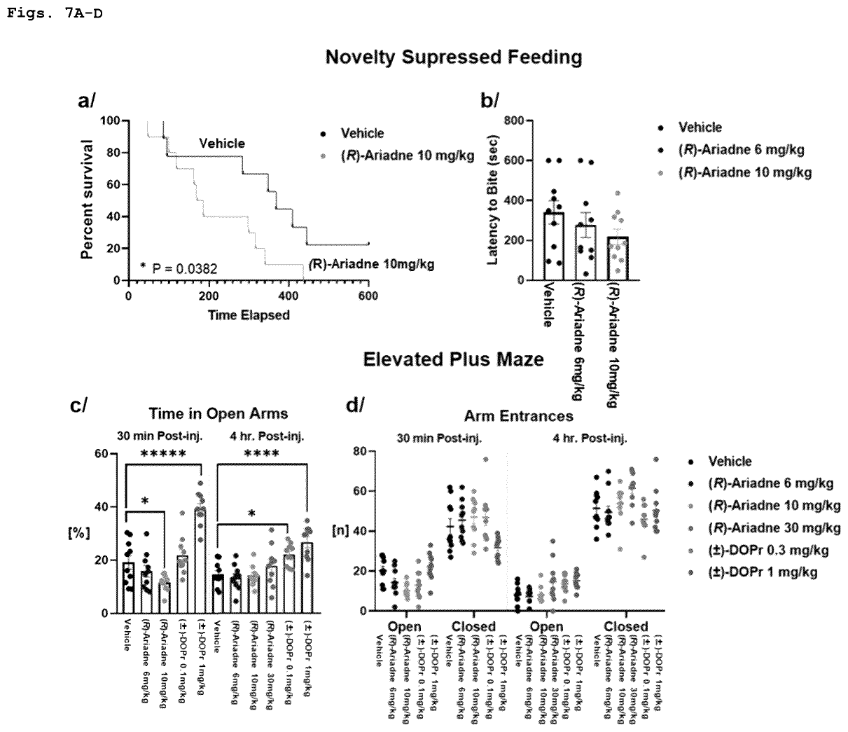

Analysis of the OF data showed a trend for spending less time in the center of the arena for both (R)-Ariadne and DOPR, consistent with decreased entries and time spent in the open arms of the elevated plus maze (EPM), indicating acute anxiogenic-like effect of R-Ariadne (FIG. 7C-D). Novelty suppressed feeding (NSF) performed in mice 7 days post-drug administration demonstrated a dose-dependent trend toward reducing latency to bite the presented food pellets at 10 mg/kg for (R)-Ariadne, suggesting a lasting anxiolytic-like effect (Samuels et al., 2020).

### Example 16 Pharmacokinetic Assessment

PK assessments were performed at Sai Life (Telangana, India) according to the following general procedure. A total twenty-seven male C57BL/6 mice were used in this study with 3 mice per time point. Animals were administered subcutaneously at 10 mg/kg dose of test article. The formulation vehicle used was 0.9% normal saline. Blood samples (approximately 60 μL) were collected under light isoflurane anesthesia (Surgivet®) from retro orbital plexus from a set of three mice at 0.08, 0.25, 0.5, 1, 2, 4, 8, 24 and 48 hr. Immediately after blood collection, plasma was harvested by centrifugation at 4000 rpm, 10 min at 40° C. and samples were stored at −70±10° C. until bioanalysis. Following blood collection, immediately animals were sacrificed followed by cutting abdominal vena-cava and whole body was perfused from heart using 10 mL of normal saline. Brain samples were collected from set of three mice at 0.08, 0.25, 0.5, 1, 2, 4, 8, 24 and 48 hr. After isolation, brain samples were rinsed three times in ice cold normal saline (for 5-10 seconds/rinse using ˜5-10 mL normal saline in disposable petri dish for each rinse) and dried on blotting paper. Brain samples were homogenized using ice-cold phosphate buffer saline (pH−7.4). Total homogenate volume was three times the tissue weight. All homogenates were stored below −70±10° C. until bioanalysis. All samples were processed for analysis by protein precipitation method and analyzed with fit-for-purpose LC-MS/MS method (LLOQ=5.16 ng/mL for plasma and 2.06 ng/mL for brain).

TABLE 3

| | | | | | AUCₗₐₛₜ | | | |
|---|---|---|---|---|---|---|---|---|
| | | Dose | Tₘₐₓ | Cₘₐₓ | (hr*ng/ | T_{1/2} | | |
| Matrix | Route | (mg/kg) | (hr) | (ng/mL) | mL) | (hr) | — | — |
| Plasma | SC | 10 | 0.25 | 801.64 | 1184.1 | 0.84 | — | — |

| | | | | | AUCₗₐₛₜ | | Brain- | Brain- |
|---|---|---|---|---|---|---|---|---|
| | | Dose | Tₘₐₓ | Cₘₐₓ | (hr*ng/ | T_{1/2} | Kp | Kp |
| Matrix | Route | (mg/kg) | (hr) | (ng/g) | g) | (hr) | (Cₘₐₓ) | (AUCₗₐₛₜ) |
| Brain | SC | 10 | 0.50 | 5606.44 | 10938. | 0.89 | 6.99 | 9.24 |

Pharmacokinetics data of rac-Ariadne in male C57BL/6 mice following a single subcutaneous administration (Dose: 10 mg/kg)

TABLE 4

| | | | Mean plasma | Mean brain | |
|---|---|---|---|---|---|
| | Dose | Time | Concentration | Concentration | Brain/Plasma |
| Route | (mg/kg) | (hr) | (ng/mL) | (ng/g) | ratio |
| SC | 10 | 0.083 | 672.11 | 1631.82 | 2.43 |
| | | 0.25 | 801.64 | 4755.16 | 5.93 |
| | | 0.5 | 661.96 | 5606.44 | 8.47 |
| | | 1 | 512.04 | 5239.35 | 10.23 |
| | | 2 | 172.44 | 1711.49 | 9.93 |
| | | 4 | 40.88 | 368.18 | 9.01 |
| | | 8 | BLQ | 16.23 | NC |
| | | 24 | BLQ | BLQ | NC |
| | | 48 | BLQ | BLQ | NC |

Mean brain-to-plasma concentration ratio of rac-Ariadne in male C57BL/6 mice following a single subcutaneous administration (Dose: 10 mg/kg)

TABLE 5

| | | | | | AUCₗₐₛₜ | | | |
|---|---|---|---|---|---|---|---|---|
| | | Dose | Tₘₐₓ | Cₘₐₓ | (hr*ng/ | T_{1/2} | | |
| Matrix | Route | (mg/kg) | (hr) | (ng/mL) | mL) | (hr) | — | — |
| Plasma | SC | 10 | 0.25 | 466.69 | 797.87 | 2.11 | — | — |

| | | | | | AUCₗₐₛₜ | | Brain- | Brain- |
|---|---|---|---|---|---|---|---|---|
| | | Dose | Tₘₐₓ | Cₘₐₓ | (hr*ng/ | T_{1/2} | Kp | Kp |
| Matrix | Route | (mg/kg) | (hr) | (ng/g) | g) | (hr) | (Cₘₐₓ) | (AUCₗₐₛₜ) |
| Brain | SC | 10 | 0.25 | 4156.2 | 10779.4 | 1.79 | 8.91 | 13.51 |

Pharmacokinetics data of 4C-TFM in male C57BL/6 mice following a single subcutaneous administration (Dose: 10 mg/kg)

TABLE 6
Mean brain

| | | | Mean plasma | Mean brain | |
|---|---|---|---|---|---|
| | Dose | Time | Concentration | Concentration | Brain/Plasma |
| Route | (mg/kg) | (hr) | (ng/mL) | (ng/g) | ratio |
| SC | 10 | 0.083 | 431.26 | 2011.16 | 4.66 |
| | | 0.25 | 466.69 | 4156.28 | 8.91 |
| | | 0.5 | 422.13 | 3770.22 | 8.93 |
| | | 1 | 215.78 | 3900.97 | 18.08 |
| | | 2 | 95.55 | 1742.53 | 18.24 |
| | | 4 | 51.87 | 787.06 | 15.17 |
| | | 8 | 13.45 | 170.36 | 12.67 |
| | | 24 | BLQ | BLQ | NC |
| | | 48 | BLQ | BLQ | NO |

Mean brain-to-plasma concentration ratio of 4C-TFM in male C57BL/6 mice following a single subcutaneous administration (Dose: 10 mg/kg)

TABLE 7

| Compound | EC₅₀ (5-HT_{2A}) (M) | EC₅₀ (5-HT_{2B}) (M) |
|---|---|---|
| 4C-D (32) | 1.8E−06 | 1.7E−06 |
| 29 | 6.3E−08 | 4.0E−07 |
| 53 | 5.7E−08 | 1.9E−07 |
| 50 | 2.5E−08 | 1.1E−07 |
| 58 | 4.3E−06 | — |

IP1 agonism summary for selected compounds (duplicate averages only), performed at Cerep, Eurofins.

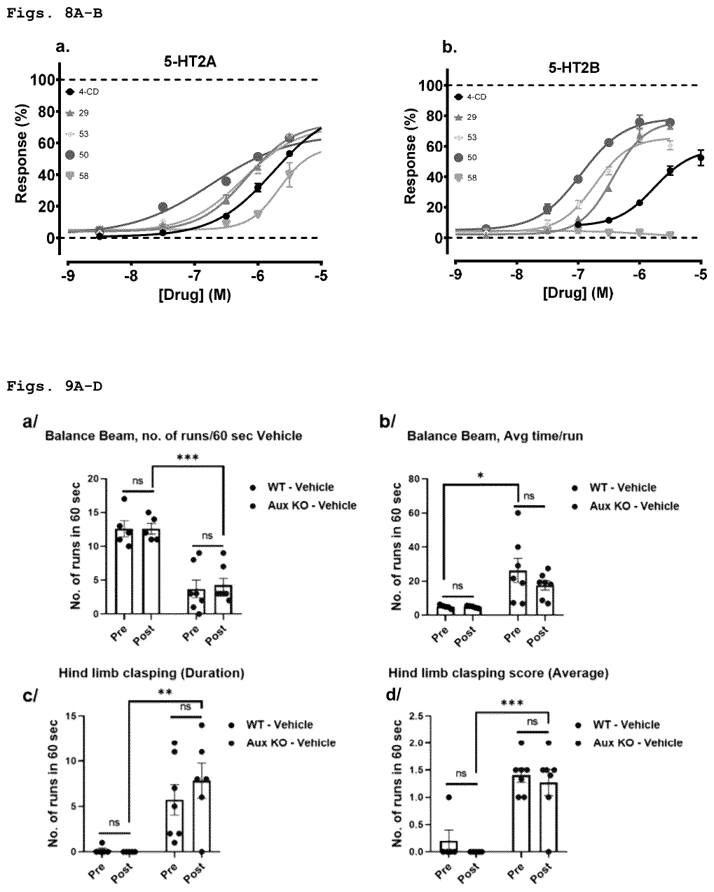

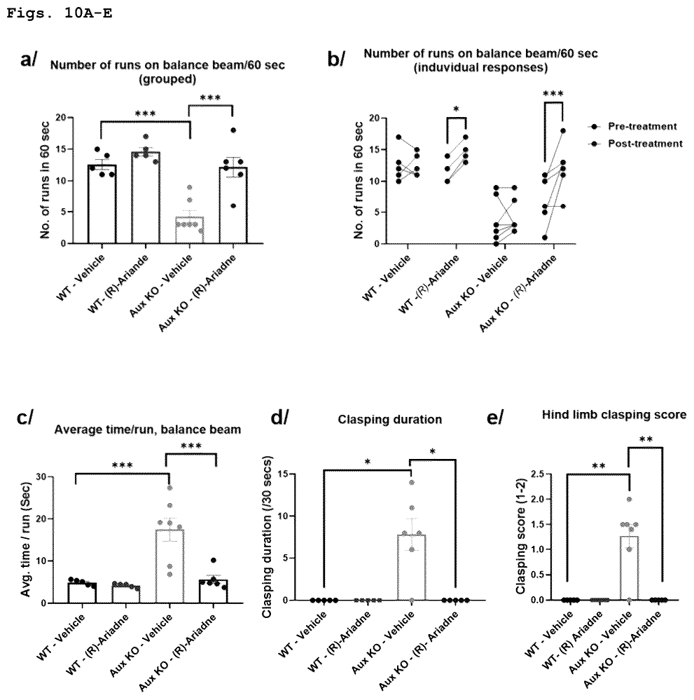

R-Ariadne ameliorates motor abnormalities in a mouse model of Parkinson's Disease. In the 1978 patent from the Bristol Laboratories, the inventors noted complete remission of Parkinson's symptoms in two patients (Partyka el al., U.S. Pat. No. 4,105,695, 1978). To address and examine these claims via reverse translation in mice, we used a novel model of PD based on auxilin knockout (KO) mice (Vidyadhara et al., 2022). Auxilin is a clathrin uncoating protein that participates in clathrin mediated endocytosis of synaptic vesicles (SV), enabling SV recycling and neurotransmission (Vidyadhara et al., 2022). Loss-of-function mutations in auxilin (DNAJC6/PARK19) cause PD. Auxilin KO mice display cardinal features of PD such as progressive motor deficits, nigral dopaminergic neuronal loss, α-synuclein pathology, and neuroinflammation. 9-12 month old Aux-KO mice were previously shown to respond to L-DOPA treatment, which ameliorated PD-like motor abnormalities (Vidyadhara et al., 2022). Here, we treated 9-12 months old Aux-KO mice with (R)-Ariadne (10 mg/kg body weight, intraperitoneal), followed by various motor behavior tests to evaluate if (R)-Ariadne can rescue the Parkinsonian phenotype akin to L-DOPA. Appropriate wildtype (WT) and vehicle controls were used. One week before the (R)-Ariadne treatment, all the mice were subjected to the balance beam, hind limb clasping and open field behavior tests (pre-treatment group). The balance beam test evaluates fine motor skills such as ability to walk on a raised narrow beam to reach a safety box, measured as a number of runs/minute and time taken to cross the beam (FIGS. 9-10). Number of runs auxilin KOs could perform in a minute were significantly lesser when compared to WT mice before (R)-Ariadne treatment. Time taken to cross the beam was also significantly higher in Aux-KO mice (FIGS. 9-10). Mice with neurological dysfunction typically show hind limb clasping reflex when picked up by the tail. Auxilin KOs show a significant increase in hind limb clasping score when compared to WTs before treatment (FIG. 9A-B). Though not significant, auxilin KOs also showed a trend toward an increase in clasping duration (FIGS. 9C,D 10C,D). Finally, to assess the overall locomotion, we performed open field test to check distance traveled in given time, which was significantly deteriorated in auxilin KOs before (R)-Ariadne treatment. In a sum, auxilin KO mice show a robust behavioral phenotype.

One week later, we repeated these behavior assays on the same set of mice 15 to 30 minutes after (R)-Ariadne treatment with appropriate vehicle controls (“Post-treatment”). After (R)-Ariadne treatment, Aux-KO mice showed a remarkable recovery on balance beam, where numbers of runs they could perform was significantly higher and comparable to WT mice (FIGS. 9A-B and 10A-B). Though not significant, a similar recovery was seen in time taken/run on the balance beam. Surprisingly, even the WT mice showed an improvement in balance beam performance as measured by number of runs per minute after (R)-Ariadne treatment, when compared to how much they could run before treatment (FIG. 10A-B). Auxilin KO mice also showed a significant recovery in hindlimb clasping behavior following (R)-Ariadne treatment. Both clasping score (and clasping duration (FIGS. 10C-E) was down to zero in Aux-KO mice after (R)-Ariadne treatment, suggesting a remarkable recovery. The overall locomotory deficits seen in the open field was partially rescued after (R)-Ariadne treatment although these results are confounded by learning effects (lack of novelty) during the repeated OF testing. Together, these results demonstrate that (R)-Ariadne treatment successfully and rapidly reversed selective motor behavior deficits seen in a PD mouse model, mirroring the effects of L-DOPA treatment, which is the current standard of care for PD patients.

### Discussion

Mechanistic hypothesis for lack of Ariadne's hallucinogenic effects—signaling efficacy hypothesis. The above results provide a molecular mechanistic picture for Ariadne and its analogs. Ariadne is a selective 5-HT2 receptor agonist, preferring the 5-HT_{2A} over 5-HT-_{2B/C} receptor subtypes, with modest selectivity over 5-HT_{1E} and 5-HT_{1F} receptors (0.5-1 log), no/low activity (EC₅₀>10 uM) at 5-HT_{5,6,7}, dopamine 1 and 2, and adrenergic receptors, and no relevant affinity at monoamine transporters. This clean pharmacological profile renders the interpretation of both the new results (acquired in the current study) and previous data relatively straightforward, in comparison to the widely known ergoline non-hallucinogens that in contrast have a very complex pharmacology (Marona-Lewicka et al., 2002). The HTR studies in mice described above indicate that Ariadne acts as a 5-HT_{2A} agonist in vivo. However, in comparison to its hallucinogenic analogs, the HTR induced by Ariadne compounds shows markedly attenuated HTR efficacy in a lower potency range. Notwithstanding the caveats in the interpretation of HTR efficacy, the results of mouse HTR are consistent with the previous studies in rabbits and cats, which demonstrated a behavioral profile distinct from the corresponding DOx amphetamine hallucinogens (Standridge et al., 1980 and Standridge et al., 1976). This preclinical behavioral profile correlates with the in vitro pharmacology where Ariadne, in comparison to DOM, shows lower signaling potency and efficacy in the four signaling pathways examined (G_{q}, G₁₁, and β-arrestin2) coupled to 5-HT_{2A} receptors. This shift in signaling was observed for two other pairs of alpha-ethyl and alpha-methyl analogs (4C-Pr vs DOPR, and 4C-I vs DOI). There is no apparent change in the relative bias of signaling between G_{q/11} and β-arrestin2, instead there is a small but consistent drop in efficacy in all signaling channels. Hence we propose the weaker 5-HT_{2A} signaling efficacy as an explanatory model for the lack of hallucinogenic effects of Ariadne in humans, and the dramatically attenuated hallucinosis-like effects of Ariadne class in animals (the signaling efficacy hypothesis).

Mechanistic hypothesis for Ariadne's clinical observations. We propose the following rationale for the rapid effects of Ariadne in the mouse PD model. The in vitro profile suggests that Ariadne's effect on dopamine neurotransmission is indirect, namely not via direct modulation of DAT or dopamine receptors. It has been demonstrated that 5-HT₂ₐ agonists increase dopamine release in nucleus accumbens and other regions of the mesolimbic system (Howell et al., 2014). It is therefore likely that 5-HT₂ₐ agonists also stimulate DA release in more dorsal areas of the striatum that are compromised by the PD pathology. The mechanism of this effect remains unclear; it is plausible that there are 5-HT₂ₐ receptors on dopamine neurons which directly stimulate dopamine release, and or there is a circuit effect where for example stimulation of PFC 5-HT₂ₐ receptors leads to an excitatory drive to the midbrain nuclei such as VTA, DRN, and others,⁴⁰ which in turn leads to compensatory stimulation of DA release in areas underpinning the motor deficits associated with PD. If this is the case, the present results indicate that non-hallucinogenic 5HT₂ₐ agonists of Ariadne class maintain this modulatory effect on the dopaminergic system. 5-HT₂ₐ receptor agonists have also been reported to increase biogenesis of mitochondria (Fanibunda et al., 2019), which suggests an additional plausible mechanism for rescue effects, particularly in terms of longer lasting effects. Our future studies will examine Ariadne's long term therapeutic-like effects in this PD model.

Emerging class of non-hallucinogenic 5-HT2A agonists. Ariadne is a prime example of a non-hallucinogenic 5-HT₂ₐ agonist with therapeutic potential based on human and clinical experience. Several other compounds of this category have been previously discovered, such as lisuride and ergotamine, which are close structural analogs of the potent classic hallucinogen LSD (Marona-Lewicka et al., 2002). Historically, non-hallucinogens have been largely overlooked as they lack the defining feature of psychedelics—the ability to induce transient altered brain-mind states. However, these compounds have the potential to become a new class of take-home psychiatric medications where different modalities of use can be envisioned. This paper presents another path for the use of these compounds in neurological disorders, such as PD, where accompanying psychiatric symptoms (such as hallucinations) presents a critical barrier.

In more recent efforts, several 5-HT₂ₐ agonists of different structural scaffolds with predicted non-hallucinogenic profiles have been introduced; such as tabernanthalog (an analog of azepinoindoles developed in the 1960's by Upjohn company) (Hester et al., 1968 and Cameron et al., 2020). Preclinical data presented for both agents is supportive of potential clinical use for depression and substance use disorders. However, these agents have not yet been examined in humans, and thus their psychedelic and therapeutic efficacy are currently unknown.

There are a number of mechanistic hypotheses proposed to explain the distinct behavioral phenotypes induced by the non-hallucinogenic agonists, ranging from low receptor engagement (e.g. due to high metabolism/clearance or dose-limiting side effects), to modulation of other targets that may inhibit the expression of psychedelic responses (e.g. 5-HT1a receptor agonism), to divergent downstream 5-HT₂ₐ-linked signaling mechanisms (e.g. G_{q} protein versus β-arrestin or different G protein pathways) (López-Giménez et al., 2017 and Kolaczynska et al., 2019). In our present work, the direct comparison of the hallucinogen DOM and non-hallucinogen Ariadne demonstrates a consistent drop in the signaling efficacy across several 5-HT2a-signaling pathways. We therefore propose a signaling efficacy hypothesis for the lack of hallucinosis induced by Ariadne.

Conclusion The case of Ariadne is a remarkable chapter in the history of neuropsychiatric drug development, one with acute importance and relevance to the current revival of psychedelic science. To date Ariadne provides the strongest support for the therapeutic potential of non-hallucinogenic 5-HT₂ₐ receptor agonists on the basis of the total available data summarized in this article. The rise and fall of this substance as a prototype of a novel medicinal class resembles the Greek myth of Ariadne, centered on a mythical princess after whom the compound was named. There are different versions of the myth, and we here add a new thread in the molecular dimension. Ariadne led Theseus out of the labyrinth, only to be abandoned by him. She was later found by Dionysus, deity of plentitude, joy and wine, to mother many children with him. The compound Ariadne, a substance of molecular of simplicity, was leading Bristol-Myers out of innovation malaise to a new type of psychiatric medication, only to be abandoned by the company. Later to be found in the current revolution of psychiatric drug discovery, and we hope, to lead the way to many new medicines of this class.

### REFERENCES

Bdioui, S.; Verdi, J.; Pierre, N.; Trinquet, E.; Roux, T.; Kenakin, T. Equilibrium Assays Are Required to Accurately Characterize the Activity Profiles of Drugs Modulating GQ-Protein-Coupled Receptors. Molecular Pharmacology 2018, 94 (3), 992-1006.
Belser, A.; Canal, C. E.; Greene, B. J.; Hartsel, J.; Nivorozhkin, A.; Palfreyman, M. G. Therapeutic phenethylamine compositions and methods of use, Feb. 24, 2022.
Buyniski J P, Smith M L, Bierwagen M E. Cardiovascular and gross behavioral effects of amphetamine, 2-amino-1-(2,5-dimethoxy-4-methylphenyl) propane (DOM) and 2-amino-1-(2,5-dimethoxy-4-methylphenyl)butane (BL-3912A) in the conscious dog. Research Communications in Chemical Pathology and Pharmacology 1974, 8 (2), 213-221.
Cameron, L. P.; Tombari, R. J.; Lu, J.; Pell, A. J.; Hurley, Z. Q.; Ehinger, Y.; Vargas, M. V.; McCarroll, M. N.; Taylor, J. C.; Myers-Turnbull, D.; Liu, T.; Yaghoobi, B.; Laskowski, L. J.; Anderson, E. I.; Zhang, G.; Viswanathan, J.; Brown, B. M.; Tjia, M.; Dunlap, L. E.; Rabow, Z. T.; Fiehn, O.; Wulff, H.; McCorvy, J. D.; Lein, P. J.; Kokel, D.; Ron, D.; Peters, J.; Zuo, Y.; Olson, D. E. A Non-Hallucinogenic Psychedelic Analogue with Therapeutic Potential. Nature 2020, 589 (7842), 474-479.
Canal, C. E.; Morgan, D. Head-Twitch Response in Rodents Induced by the Hallucinogen 2,5-Dimethoxy-4-Iodoamphetamine: A Comprehensive History, a Re-Evaluation of Mechanisms, and Its Utility as a Model. Drug Testing and Analysis 2012, 4 (7-8), 556-576.
Collins, G. T.; Gerak, L. R.; France, C. P. The Behavioral Pharmacology and Therapeutic Potential of Lorcaserin for Substance Use Disorders. Neuropharmacology 2018, 142, 63-71.
Cunningham, M. J.; Bock, H. A.; Serrano, I. C.; Bechand, B.; Vidyadhara, D. J.; Bonniwell, E. M.; Lankri, D.; Duggan, P.; Nazarova, A. L.; Cao, A. B.; Calkins, M. M.; Khirsariya, P.; Hwu, C.; Katritch, V.; Chandra, S. S.; McCorvy, J. D; Sames, D. ACS Chemical Neuroscience 2023 14, (1), 119-135
Fanibunda, S. E.; Deb, S.; Maniyadath, B.; Tiwari, P.; Ghai, U.; Gupta, S.; Figueiredo, D.; Weisstaub, N.; Gingrich, J. A.; Vaidya, A. D.; Kolthur-Seetharam, U.; Vaidya, V. A. Serotonin Regulates Mitochondrial Biogenesis and Function in Rodent Cortical Neurons via the 5-HT2a Receptor and SIRT1-Pgc-1α Axis. Proceedings of the National Academy of Sciences 2019, 116 (22), 11028-11037.
Gewirtz, J. C.; Marek, G. J. Behavioral Evidence for Interactions between a Hallucinogenic Drug and Group II Metabotropic Glutamate Receptors. Neuropsychopharmacology 2000, 23 (5), 569-576.
Glennon, R. A. MDMA-like Stimulus Effects of α-Ethyltryptamine and the α-Ethyl Homolog of Dom. Pharmacology Biochemistry and Behavior 1993, 46 (2), 459-462.
Halberstadt, A. L.; Chatha, M.; Chapman, S. J.; Brandt, S. D. Comparison of the Behavioral Effects of Mescaline Analogs Using the Head Twitch Response in Mice. Journal of Psychopharmacology 2019, 33 (3), 406-414.
Halberstadt, A. L.; Chatha, M.; Klein, A. K.; Wallach, J.; Brandt, S. D. Correlation between the Potency of Hallucinogens in the Mouse Head-Twitch Response Assay and Their Behavioral and Subjective Effects in Other Species. Neuropharmacology 2020, 167, 107933.
Halberstadt, A. L.; Geyer, M. A. Effect of Hallucinogens on Unconditioned Behavior. Behavioral Neurobiology of Psychedelic Drugs 2016, 159-199.
Hester, J. B.; Tang, A. H.; Keasling, H. H.; Veldkamp, W. Azepinoindoles. I. Hexahydroazepino[4,5-B]Indoles. Journal of Medicinal Chemistry 1968, 11 (1), 101-106.
Hofmann, A. LSD, my problem child: Reflections on sacred drugs, mysticism, and science; MAPS, Multidisciplinary Association for Psychedelic Studies: Santa Cruz, CA, 2009.
Howell, L. L.; Cunningham, K. A. Serotonin 5-HT2 Receptor Interactions with Dopamine Function: Implications for Therapeutics in Cocaine Use Disorder. Pharmacological Reviews 2014, 67 (1), 176-197.
Kaleida. bridging the gap. https://erowid.org/experiences/exp.php?ID=113681 (accessed Sep. 14, 2022).
Karlsen, M.; Liu, H. L.; Berg, T.; Johansen, J. E.; Hoff, B. H. Synthesis of [13C6]-Labelled Phenethylamine Derivatives for Drug Quantification in Biological Samples. Journal of Labelled Compounds and Radiopharmaceuticals 2014, 57 (5), 378-387.
Klein, A. K.; Chatha, M.; Laskowski, L. J.; Anderson, E. I.; Brandt, S. D.; Chapman, S. J.; McCorvy, J. D.; Halberstadt, A. L. Investigation of the Structure-Activity Relationships of Psilocybin Analogues. ACS Pharmacology & Translational Science 2020, 4 (2), 533-542.
Kolaczynska, K. E.; Luethi, D.; Trachsel, D.; Hoener, M. C.; Liechti, M. E. Receptor Interaction Profiles of 4-Alkoxy-Substituted 2,5-Dimethoxyphenethylamines and Related Amphetamines. Frontiers in Pharmacology 2019, 10.
Layton, M. E.; Morales, C. A.; Shair, M. D. Biomimetic Synthesis of (−)-Longithorone a. Journal of the American Chemical Society 2002, 124 (5), 773-775.
López-Giménez, J. F.; González-Maeso, J. Hallucinogens and Serotonin 5-HT2A Receptor-Mediated Signaling Pathways. Behavioral Neurobiology of Psychedelic Drugs 2017, 45-73.
Marona-Lewicka, D.; Kurrasch-Orbaugh, D.; Selken, J.; Cumbay, M.; Lisnicchia, J.; Nichols, D. Re-Evaluation of Lisuride Pharmacology: 5-Hydroxytryptamine 1A Receptor-Mediated Behavioral Effects Overlap Its Other Properties in Rats. Psychopharmacology 2002, 164 (1), 93-107.
Molander, G. A.; Ellis, N. Organotrifluoroborates: Protected Boronic Acids That Expand the Versatility of the Suzuki Coupling Reaction. Accounts of Chemical Research 2007, 40 (4), 275-286.
Nichols, D. E. Chemistry and Structure-Activity Relationships of Psychedelics. Behavioral Neurobiology of Psychedelic Drugs 2017, 1-43.
Nichols, D. E. Psychedelics. Pharmacological Reviews 2016, 68 (2), 264-355.
Nichols, D. E.; Frescas, S.; Marona-Lewicka, D.; Huang, X.; Roth, B. L.; Gudelsky, G. A.; Nash, J. F. 1-(2,5-Dimethoxy-4-(Trifluoromethyl)Phenyl)-2-Aminopropane: A Potent Serotonin 5-HT2A/2C Agonist. Journal of Medicinal Chemistry 1994, 37 (25), 4346-4351.
Partyka, R. A.; Standridge, R. T.; Howell, H. G.; Shulgin, A. T. 2-Amino-1-(2,5-dimethoxyphenyl)butanes, U.S. Pat. No. 4,105,695, 1978.
Peng, Y.; McCorvy, J. D.; Harpsøe, K.; Lansu, K.; Yuan, S.; Popov, P.; Qu, L.; Pu, M.; Che, T.; Nikolajsen, L. F.; Huang, X.-P.; Wu, Y.; Shen, L.; Bjørn-Yoshimoto, W. E.; Ding, K.; Wacker, D.; Han, G. W.; Cheng, J.; Katritch, V.; Jensen, A. A.; Hanson, M. A.; Zhao, S.; Gloriam, D. E.; Roth, B. L.; Stevens, R. C.; Liu, Z.-J. 5-HT2C Receptor Structures Reveal the Structural Basis of GPCR Polypharmacology. Cell 2018, 172 (4).
Psychestim. Mild, But Sweet. https://erowid.org/experiences/exp.php?ID=116183 (accessed Sep. 14, 2022).
Rodriguiz, R. M.; Nadkarni, V.; Means, C. R.; Pogorelov, V. M.; Chiu, Y.-T.; Roth, B. L.; Wetsel, W. C. LSD-Stimulated Behaviors in Mice Require β-Arrestin 2 but Not β-Arrestin 1. Scientific Reports 2021, 11 (1).
Rothman, R. B.; Baumann, M. H. Serotonergic Drugs and Valvular Heart Disease. Expert Opinion on Drug Safety 2009, 8 (3), 317-329.
Samuels, B. A.; Hen, R. Novelty-Suppressed Feeding in the Mouse. Mood and Anxiety Related Phenotypes in Mice 2011, 107-121.
Seo, S.; Taylor, J. B.; Greaney, M. F. Silver-Catalysed Trifluoromethylation of Arenes at Room Temperature. Chemical Communications 2013, 49 (57), 6385.
Shakhnovich, V. It's Time to Reverse Our Thinking: The Reverse Translation Research Paradigm. Clinical and Translational Science 2018, 11 (2), 98-99.
Shulgin, A. T. The ‘social-chemistry’ of pharmacological discovery. Social Pharmacology, 1987, 1 (3), 279-290.
Shulgin, A. T.; Shulgin, A. PIHKAL: A chemical love story; Transform Press: Berkeley, CA, 2020.
Shulgin, A. T. Treatment of senile geriatric patients to restore performance. U.S. Pat. No. 4,034,113, 5 Jul. 1977.
Snyder, S. H.; Faillace, L.; Hollister, L. 2,5-Dimethoxy-4-Methyl-Amphetamine (STP): A New Hallucinogenic Drug. Science 1967, 158 (3801), 669-670.
Standridge, R. T.; Howell, H. G.; Gylys, J. A.; Partyka, R. A.; Shulgin, A. T. Phenylalkylamines with Potential Psychotherapeutic Utility. 1. 2-Amino-1-(2,5-Dimethoxy-4-Methylphenyl)Butane. Journal of Medicinal Chemistry 1976, 19 (12), 1400-1404.
Standridge, R. T.; Howell, H. G.; Tilson, H. A.; Chamberlain, J. H.; Holava, H. M.; Gylys, J. A.; Partyka, R. A.; Shulgin, A. T. Phenylalkylamines with Potential Psychotherapeutic Utility. 2. Nuclear Substituted 2-Amino-1-Phenylbutanes. Journal of Medicinal Chemistry 1980, 23 (2), 154-162.
Tilson, H. A.; Chamberlain, J. H.; Gylys, J. A. Behavioral Comparisons of R-2-Amino-1-(2,5-Dimethoxy-4-Methylphenyl) Butane (BL-3912A) with R-Dom and S-Amphetamine. Psychopharmacology 1977, 51 (2), 169-173.
Vidyadhara, D. J.; Somayaji, M.; Wade, N.; Yücel, B.; Zhao, H.; Shashaank, N.; Ribaudo, J.; Gupta, J.; Lam, T. K. T.; Sames, D.; Greene, L. E.; Sulzer, D. L.; Chandra, S. S. Dopamine Transporter and Synaptic Vesicle Sorting Defects Initiate Auxilin-Linked Parkinson's Disease. BioArxiv 2022.
Weingartner, H.; Snyder, S. H.; Faillace, L. A. Dom (STP), a New Hallucinogenic Drug: Specific Perceptual Changes. The Journal of Clinical Pharmacology and New Drugs 1971, 11 (2), 103-111.
Winter, J. C. Effects of the Phenethylamine Derivatives, BL-3912, Fenfluramine, and SCH-12679, in Rats Trained with LSD as a Discriminative Stimulus. Psychopharmacology 1980, 68 (2), 159-162.
Xorkoth. Zen in a glittery powder. https://erowid.org/experiences/exp.php?ID=113083 (accessed Sep. 14, 2022).

1. A method of treating a subject afflicted with a movement disorder, comprising administering to the subject a compound having the structure: 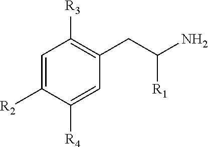
wherein
R₁ is —(C₂-C₁₂ alkyl), -(alkenyl), -(alkynyl), -(cycloalkyl);
R₂ is H, halogen, —NO₂, —CN, —CF₃, —CF₂H, —CH₂OCH₃, —CF₂CH₃, —CF₂OCH₃, -(alkyl), -(alkenyl), -(alkynyl), -(cycloalkyl), -(cycloalkylalkyl), -(heteroalkyl), -(heterocycle), -(heterocycloalkyl), -(aryl), -(heteroaryl), -(hydroxyalkyl), -(haloalkyl), -(alkylaryl), —OH, —O-(alkyl), —O-(alkenyl), —O-(alkynyl), —O-(haloalkyl), —O-(aryl), —O-(heteroaryl), —OCF₃, SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —N-(alkyl)₂, NH-(aryl), —NH-(heteroaryl), —CO₂H, —CO₂-(alkyl), —C(O)—NH₂, —C(O)—NH-(alkyl), —C(O)—NH-(aryl), —SO₂CH₃ or —Si(CH₃)₃;
R₃ is —OCH₃, —OCH₂CH₃, —F or —Cl; and
R₄ is —OCH₃, —OCH₂CH₃ or —SCH₃;
wherein when R₁ is —CH₂CH₃, R₃ is —OCH₃, and R₄ is —OCH₃, then R₂ is other than H, —CH₃, —CH₂CH₃, —CH₂CH₂CH₃, —CH(CH₃)₂, —CH₂CH₂CH₂CH₃, —CH₂OH, —CH(OH)CH₃, —OH, —OCH₂CH₃, —OCH₂CH₂CH₃, —OCH(CH₃)₂, —SCH₃, —SCH₂CH₃, —SCH₂CH₂CH₃, —NO₂, —NH₂, —F, —Cl, —Br or —I,
or a pharmaceutically acceptable salt thereof, in an amount effective to ameliorate a locomotor deficit in the subject.

2. (canceled)

3. The method of claim 1, wherein
(a) the movement disorder comprises Parkinson's disease or catatonia;
(b) the subject is further afflicted with dementia, schizophrenia, or bipolar disorder;
(c) the locomotor deficit comprises a deficit in fine motor skills, a deficit in balance, or ataxia; and/or
(d) the compound is effective to reverse the locomotor deficit.

4-6. (canceled)

7. The method of claim 1, wherein in the compound
R₁ is —(C₂-C₁₂ alkyl), -(alkenyl), -(alkynyl), -(cycloalkyl);
R₂ is H, halogen, —NO₂, —CN, —CF₃, —CF₂H, -(alkyl), -(alkenyl), -(alkynyl), -(cycloalkyl), -(cycloalkylalkyl), -(heteroalkyl), -(heterocycle), -(heterocycloalkyl), -(aryl), -(heteroaryl), -(hydroxyalkyl), -(haloalkyl), -(alkylaryl), —OH, —O-(alkyl), —O-(alkenyl), —O-(alkynyl), —O-(haloalkyl), —O-(aryl), —O-(heteroaryl), —OCF₃, SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —N-(alkyl)₂, NH-(aryl), —NH-(heteroaryl), —CO₂H, —CO₂-(alkyl), —C(O)—NH₂, —C(O)—NH-(alkyl), —C(O)—NH-(aryl) or —Si(CH₃)₃;
R₃ is —OCH₃, —F or —Cl; and
R₄ is —OCH₃ or —SCH₃;
wherein when R₁ is —CH₂CH₃, R₃ is —OCH₃, and R₄ is —OCH₃, then R₂ is other than H, —CH₃, —CH₂CH₃, —CH₂CH₂CH₃, —CH(CH₃)₂, —CH₂CH₂CH₂CH₃, —CH₂OH, —CH(OH) CH₃, —OH, —OCH₂CH₃, —OCH₂CH₂CH₃, —OCH(CH₃)₂, —SCH₃, —SCH₂CH₃, —SCH₂CH₂CH₃, —NO₂, —NH₂, —F, —Cl, —Br or —I.

8. (canceled)

9. The method of claim 1, wherein in the compound
R₁ is —(C₂-C₁₂ alkyl), -(alkenyl), -(alkynyl), -(cycloalkyl);
R₂ is H, —CN, —CF₃, —CH₂OCH₃, —CF₂CH₃, —CF₂OCH₃, -(alkyl), -(alkenyl), -(alkynyl), -(cycloalkyl), -(heterocycle), —S-(aryl), —SO₂CH₃ or —Si(CH₃)₃;
R₃ is —OCH₃, —OCH₂CH₃, —F or —Cl; and
R₄ is —OCH₃, —OCH₂CH₃ or —SCH₃.

10. The method of claim 1, wherein in the compound
R₁ is —(C₂-C₁₂ alkyl), -(alkenyl), -(alkynyl), -(cycloalkyl);
R₂ is H, —CN, —CF₃, -(alkyl), -(alkenyl), -(alkynyl), -(cycloalkyl), -(heterocycle), —S-(aryl), or —Si(CH₃)₃;
R₃ is —OCH₃, —F or —Cl; and
R₄ is —OCH₃, or —SCH₃.

11. The method of claim 10, wherein in the compound
R₁ is —(C₃-C₁₂ alkyl), -(alkenyl), -(alkynyl), -(cycloalkyl);
R₂ is H, —CN, —CF₃, -(alkyl), -(alkenyl), -(alkynyl), -(cycloalkyl), -(heterocycle), —S-(aryl) or —Si(CH₃)₃;
R₃ is —OCH₃, —F or —Cl; and
R₄ is —OCH₃ or —SCH₃.

12. The method of claim 11 wherein in the compound
(a) R₁ is —CH₂CH₃, —CH₂CH₂CH₃, —CH₂CH═CH₂ or cyclopropyl,
preferably wherein R₁ is —CH₂CH₂CH₃, —CH₂CH═CH₂ or cyclopropyl;
(b) R₂ is —CN, —CF₃, —CF₂H, —CH₂OCH₃, —CF₂CH₃, —CF₂OCH₃, —CH₃, —CCH, -cyclopropyl, -cyclobutyl, 2-oxetanyl, —S-(phenyl), —SO₂CH₃ or —Si(CH₃)₃, preferably, R₂ is —CN, —CF₃, —CH₃, —CCH, -cyclopropyl, -cyclobutyl, 2-oxetanyl, —S-(phenyl) or —Si(CH₃)₃;
(c) R₃ is —OCH₃, —OCH₂CH₃, —F or —Cl; and/or
(d) R₄ is —OCH₃, —OCH₂CH₃, or —SCH₃.

13-18. (canceled)

19. The method of claim 1, wherein in the compound
R₁ is —CH₂CH₃, —CH₂CH₂CH₃, —CH₂CH═CH₂ or cyclopropyl;
R₂ is —CN, —CF₃, —CH₂OCH₃, —CF₂CH₃, —CF₂OCH₃, —CH₃, —CCH, -cyclopropyl, -cyclobutyl, 2-oxetanyl, —S-(phenyl), —SO₂CH₃ or —Si(CH₃)₃;
R₃ is —OCH₃, —OCH₂CH₃, F or Cl, and
R₄ is —OCH₃, —OCH₂CH₃ or —SCH₃, or
wherein
R₁ is —CH₂CH₃, —CH₂CH₂CH₃, —CH₂CH═CH₂ or cyclopropyl;
R₂ is —CN, —CF₃, —CH₃, —CCH, -cyclopropyl, -cyclobutyl, 2-oxetanyl, —S-(phenyl) or —Si(CH₃)₃;
R₃ is —OCH₃, F or Cl, and
R₄ is —OCH₃ or —SCH₃, or
wherein
R₁ is —CH₂CH₂CH₃, —CH₂CH═CH₂ or cyclopropyl;
R₂ is —CN, —CF₃, —CH₂OCH₃, —CF₂CH₃, —CF₂OCH₃, —CH₃, —CCH, -cyclopropyl, -cyclobutyl, 2-oxetanyl, —S-(phenyl), —SO₂CH₃ or —Si(CH₃)₃;
R₃ is —OCH₃, —OCH₂CH₃, F or Cl, and
R₄ is —OCH₃, —OCH₂CH₃, or —SCH₃, or
wherein
R₁ is —CH₂CH₂CH₃, —CH₂CH═CH₂ or cyclopropyl;
R₂ is —CN, —CF₃, —CH₃, —CCH, -cyclopropyl, -cyclobutyl, 2-oxetanyl, —S-(phenyl) or —Si(CH₃)₃;
R₃ is —OCH₃, F or Cl, and
R₄ is —OCH₃ or —SCH₃.

20. The method of claim 19, wherein the compound has the structure: 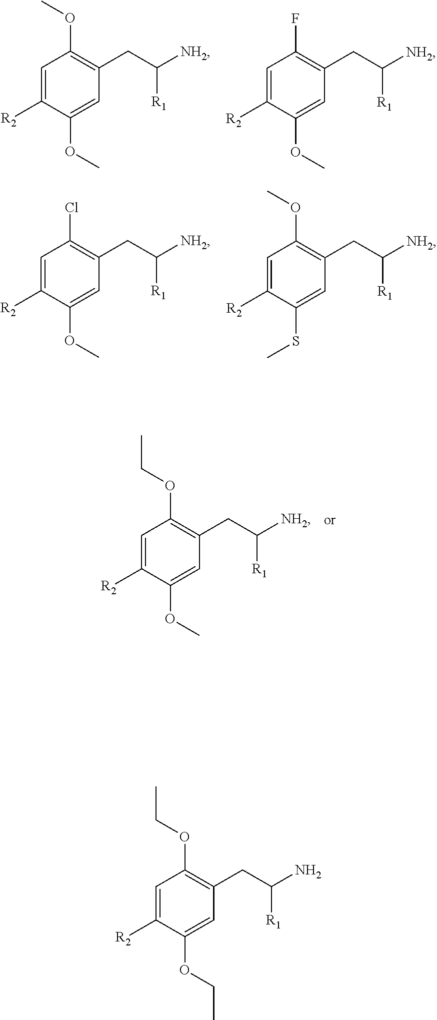
or a pharmaceutically acceptable salt thereof.

21-22. (canceled)

23. The method of claim 1, wherein in the compound
(a) R₁ is —CH₂CH₃, —CH₂CH₂CH₃, —CH₂CH═CH₂ or cyclopropyl; or
(b) R₂ is H, —CN, —CF₃, -(alkyl), -(alkenyl), -(alkynyl), -(cycloalkyl), -(heterocycle), —S-(aryl) or —Si(CH₃)₃;
preferably, R₂ is —CN, —CF₃, —CH₃, —CCH, -cyclopropyl, -cyclobutyl, 2-oxetanyl, —S-(phenyl) or —Si(CH₃)₃; more preferably, R₂ is —CF₃, —CH₂OCH₃ or —CF₂OCH₃.

24-26. (canceled)

27. The method of claim 1, wherein in the compound
(a) R₂ is —CN or —CF₃,
(b) R₁ is —CH₂CH₃, —CH₂CH₂CH₃, —CH₂CH═CH₂ or cyclopropyl; and
R₂ is —CN or —CF₃,
(c) R₁ is —CH₂CH₃, and R₂ is —CN or —CF₃,
(d) R₁ is —CH₂CH₂CH₃, and R₂ is —CN or —CF₃,
(e) R₁ is —CH₂CH═CH₂, and R₂ is —CN or —CF₃,
(f) R₁ is cyclopropyl, and R₂ is —CN or —CF₃;
(g) R₂ is —CF₃, —CH₂OCH₃, —CF₂CH₃, —CF₂OCH₃ or —SO₂CH₃, or
(h) R₁ is —CH₂CH₃, —CH₂CH₂CH₃, —CH₂CH═CH₂ or cyclopropyl; and
R₂ is —CF₃, —CH₂OCH₃, —CF₂CH₃, —CF₂OCH₃ or —SO₂CH₃,
(i) R₁ is —CH₂CH₃, and R₂ is CF₃, —CH₂OCH₃, —CF₂CH₃, —CF₂OCH₃ or —SO₂CH₃,
(j) R₁ is —CH₂CH₂CH₃, and R₂ is CF₃, —CH₂OCH₃, —CF₂CH₃, —CF₂OCH₃ or —SO₂CH₃,
(k) R₁ is —CH₂CH═CH₂, and R₂ is CF₃, —CH₂OCH₃, —CF₂CH₃, —CF₂OCH₃ or —SO₂CH₃, or
(l) R₁ is cyclopropyl, and R₂ is CF₃, —CH₂OCH₃, —CF₂CH₃, —CF₂OCH₃ or —SO₂CH₃.

28. (canceled)

29. The method of claim 1, wherein in the compound
R₁ is —CH₂CH₃; and
R₂ is —CF₃, —CH₂OCH₃ or —CF₂OCH₃.

30. The method of claim 1, wherein the compound has the structure: 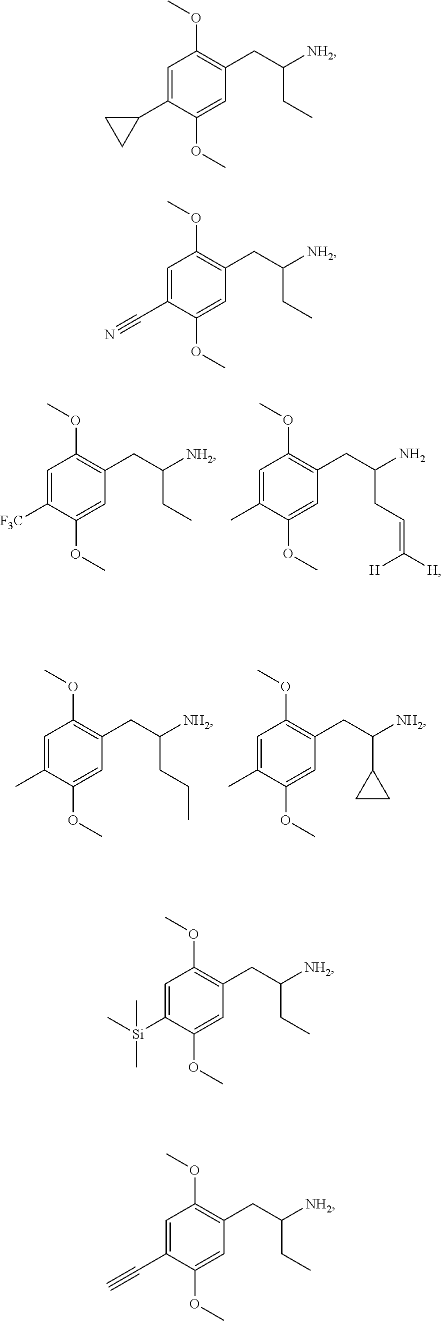 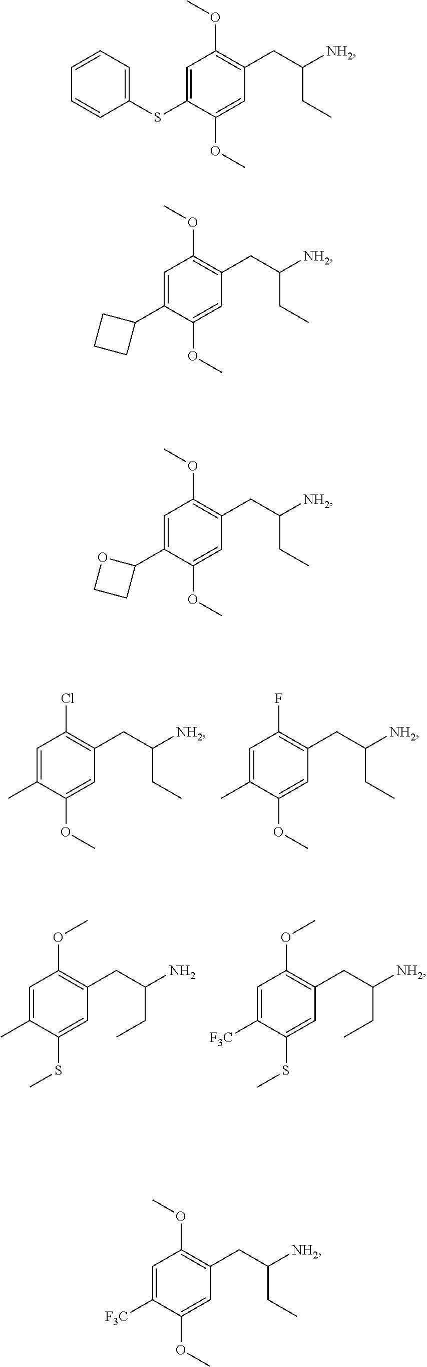 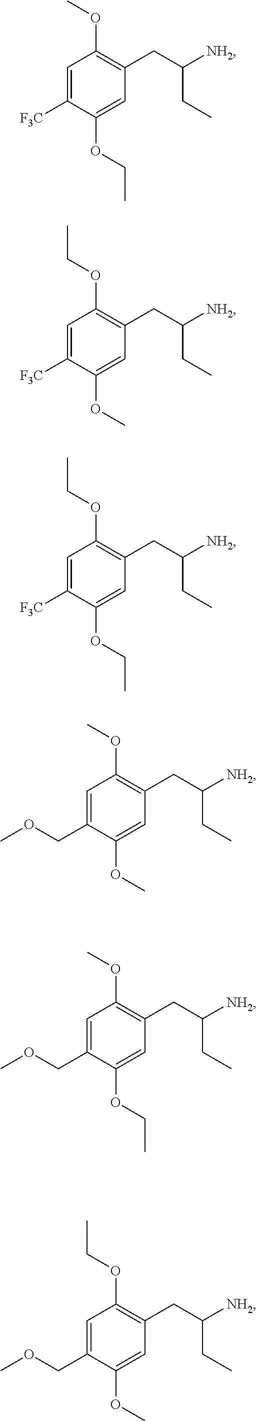 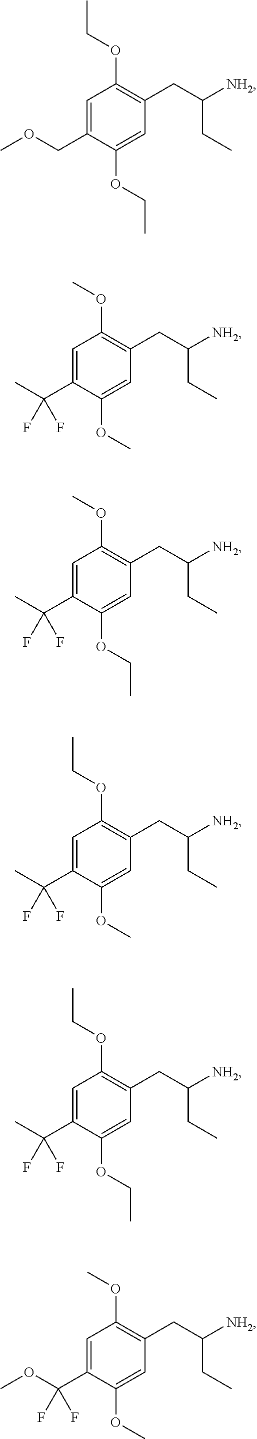 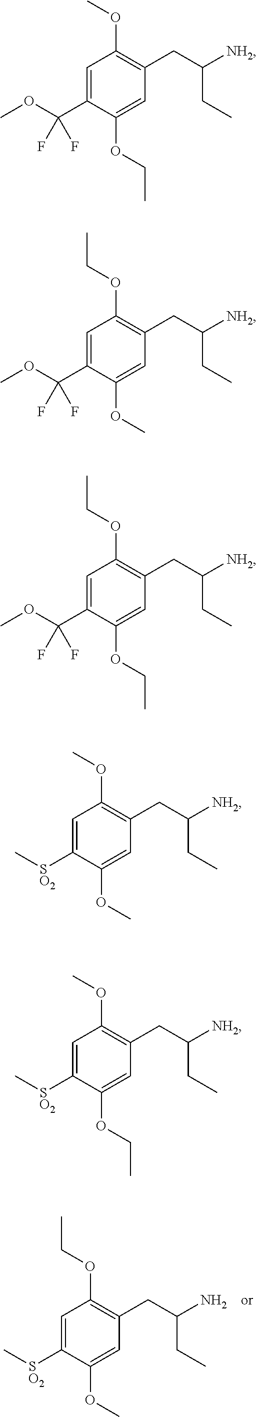 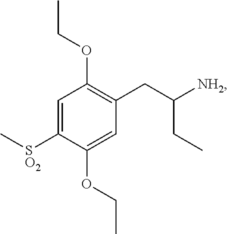 [SMILES: CCOc1cc(S(C)(=O)=O)c(OCC)cc1CC(N)CC]
or a pharmaceutically acceptable salt thereof.

31. (canceled)

32. The method of claim 1, wherein in the compound
(a) R₁ is —CH₂CH₃,
R₂ is —CF₃, —CH₂OCH₃ or —CF₂OCH₃,
R₃ is —OCH₃; and
R₄ is —OCH₃; or
(b) R₁ is —CH₂CH₃;
R₂ is —CHFCH₃, —CF₂CH₃, —CHFCH₂CH₃, —CH₂CHFCH₃, —CF₂CH₂CH₃, —CH₂CF₂CH₃, —CHFCH₂CH₂CH₃, —CH₂CFHCH₂CH₃, —CH₂CH₂CHFCH₃, —CF₂CH₂CH₂CH₃, —CH₂CF₂CH₂CH₃, or —CH₂CH₂CF₂CH₃;
R₃ is —OCH₃; and
R₄ is —OCH₃.

33. (canceled)

34. The method of claim 1, wherein the compound has the structure: 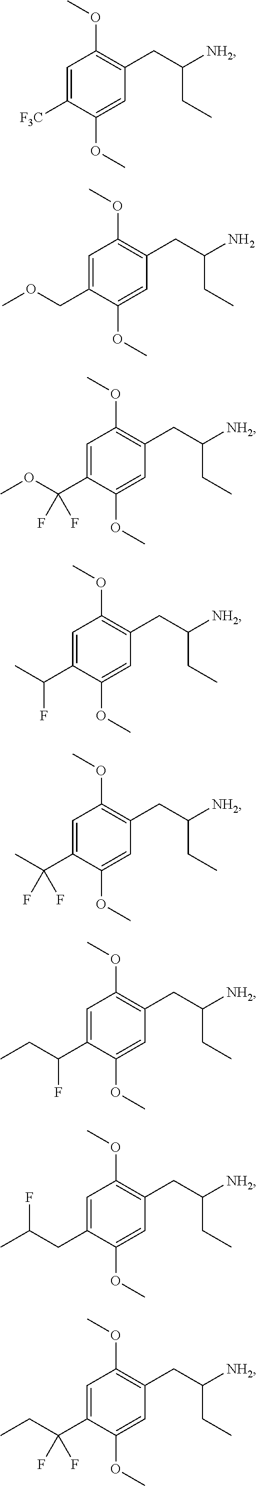 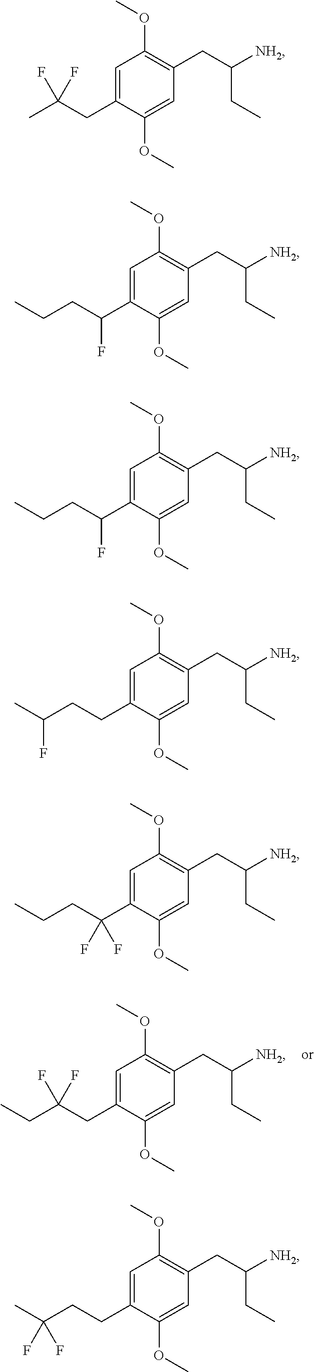
or a pharmaceutically acceptable salt thereof,
or a mixture of enantiomers, single enantiomer or diastereomer thereof,
or a pharmaceutically acceptable salt of a mixture of enantiomers, single enantiomer or diastereomer thereof
or a pharmaceutically acceptable salt thereof.

35. (canceled)

36. The method of claim 1, wherein the compound has the structure: 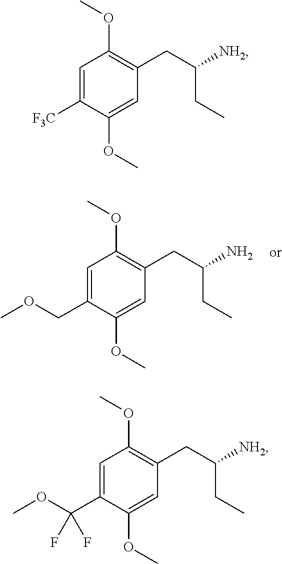
or a pharmaceutically acceptable salt thereof.

37. The method of claim 1, wherein
(a) the compound is administered in a pharmaceutical composition comprising the compound and a pharmaceutically acceptable carrier;
(b) administering the compound activates 5HT2A receptor,
preferably wherein the method selectively activates the 5HT2A receptor compared to the 5HT2B receptor;
(c) the subject is further afflicted with dementia, Alzheimer's disease, attention deficit hyperactivity disorder (ADHD), schizophrenia, depression, bipolar disorder, anxiety disorder, obsessive-compulsive disorder (OCD), stress disorder, a substance use disorder, opioid withdrawal symptoms, cluster headache, diabetic retinopathy, dry eyes, macular degeneration or glaucoma, or wherein alertness and or ability to learn in the subject is enhanced;
(d) the effective amount of the compound administered to the subject does not induce a stimulant effect, or wherein the effective amount of the compound administered to the subject does not induce a hallucinogenic effect, or wherein the effective amount of the compound administered to the subject does not induce a stimulant effect and a hallucinogenic effect,
preferably wherein the subject is a mammal,
more preferably wherein the mammal is a human; and/or
(e) the effective amount is between about 25 and about 500 mg of the compound, or
wherein the effective amount is between about 100 and 300 mg of the compound, or
wherein the effective amount is between about 0.1-20 mg/kg of compound per kilogram of body weight.

38-41. (canceled)

42. The method of claim 37, wherein the compound has the structure:

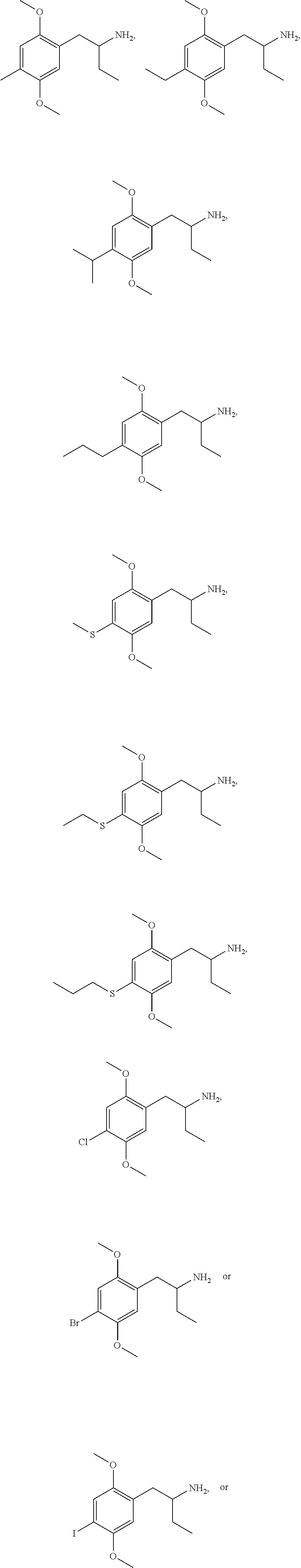

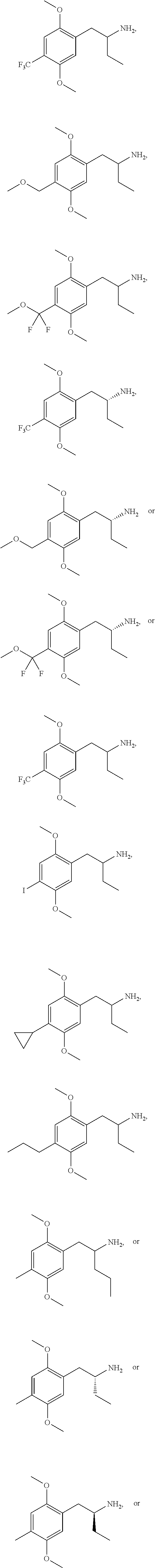

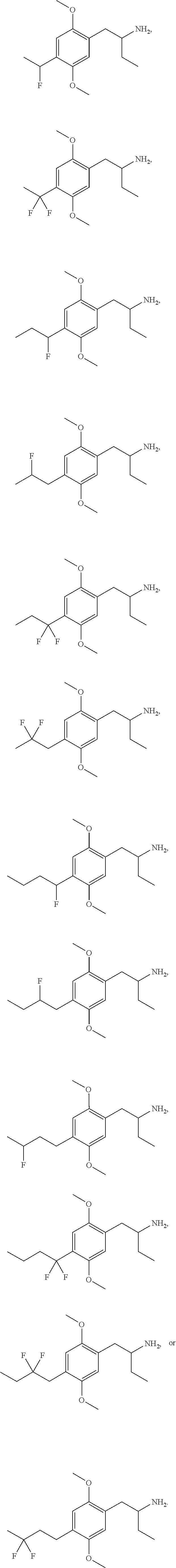

43. A compound having the structure: 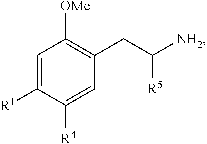
wherein
R₁ is —(C₂-C₁₂ alkyl), -(alkenyl), -(alkynyl), -(cycloalkyl)
R₂ is H, halogen, —NO₂, —CN, —CF₃, —CF₂H, —CH₂OCH₃, —CF₂CH₃, —CF₂OCH₃, -(alkyl), -(alkenyl), -(alkynyl), -(cycloalkyl), -(cycloalkylalkyl), -(heteroalkyl), -(heterocycle), -(heterocycloalkyl), -(aryl), -(heteroaryl), -(hydroxyalkyl), -(haloalkyl), -(alkylaryl), —OH, —O-(alkyl), —O-(alkenyl), —O-(alkynyl), —O-(haloalkyl), —O-(aryl), —O-(heteroaryl), —OCF₃, SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —N-(alkyl)₂, NH-(aryl), —NH-(heteroaryl), —CO₂H, —CO₂-(alkyl), —C(O)—NH₂, —C(O)—NH-(alkyl), —C(O)—NH-(aryl), —SO₂CH₃ or —Si(CH₃)₃;
R₃ is —OCH₃, —OCH₂CH₃, —F or —Cl; and
R₄ is —OCH₃, —OCH₂CH₃ or —SCH₃;
wherein when R₁ is —CH₂CH₃, R₃ is —OCH₃, and R₄ is —OCH₃, then R₂ is other than H, —CH₃, —CH₂CH₃, —CH₂CH₂CH₃, —CH(CH₃)₂, —CH₂CH₂CH₂CH₃, —CH₂OH, —CH(OH)CH₃, —OH, —OCH₂CH₃, —OCH₂CH₂CH₃, —OCH(CH₃)₂, —SCH₃, —SCH₂CH₃, —SCH₂CH₂CH₃, —NO₂, —NH₂, —F, —Cl, —Br or —I,
or a pharmaceutically acceptable salt thereof.

44. A pharmaceutical composition comprising a compound of claim 43 and a pharmaceutically acceptable carrier.
